(19) United States
(12) Patent Application Publication
(10) Pub. No.: US 2026/0002000 A1
(43) Pub. Date: Jan. 1, 2026

(54) HYBRID POLYMERS AND COMPOSITIONS INCLUDING THE SAME

(71) Applicant: REGENTS OF THE UNIVERSITY OF MINNESOTA, Minneapolis, MN (US)

(72) Inventors: Theresa M. Reineke, Vadnais Heights, MN (US); Punarbasu Roy, Coochbehar (IN); Nicholas W. Kreofsky, St. Paul, MN (US)

(21) Appl. No.: 19/250,969

(22) Filed: Jun. 26, 2025

Related U.S. Application Data
(60) Provisional application No. 63/664,835, filed on Jun. 27, 2024.

(51) Int. Cl. C08G 81/02; A61K 48/00

(52) U.S. Cl. CPC C08G 81/025; A61K 48/0041

(57) ABSTRACT

A hybrid polymer may include a hydrophilic block polymerized from a hydrophilic monomer and a hydrophobic block polymerized from at least a quinoline-containing monomer. Polyplex transfection complexes that include a hybrid polymer and an oligonucleotide. Compositions that include a polyplex transfection complex. Methods of using a composition to transfect a cell.

### CROSS-REFERENCE TO RELATED APPLICATION

This application claims the benefit of U.S. Provisional Patent Application No. 63/664,835, filed Jun. 27, 2024 which is incorporated herein by reference in its entirety.

### GOVERNMENT FUNDING

This invention was made with government support under DMR1904853 awarded by the National Science Foundation. The government has certain rights in the invention.

### SUMMARY

In one aspect, the present disclosure describes hybrid polymers. The hybrid polymers include a hydrophilic block polymerized from a hydrophilic monomer and a hydrophobic block polymerized from at least a quinoline-containing monomer.

In one or more embodiments, the hydrophobic block is polymerized from the quinoline-containing monomer and a second monomer. In one or more embodiments, the second monomer is an acrylate, methacrylate, acrylamide, methacrylamide, or vinyl monomer. In one or more embodiments, the second monomer is 2-hydroxyethyl acrylate.

In one or more embodiments, the hydrophilic block includes polyethylene glycol.

In one or more embodiments, the hydrophilic block is polymerized from at least a carbohydrate-containing monomer.

In another aspect, the present disclosure describes compositions that include a polyplex transfection complex and a carrier. A polyplex transfection complex includes a hybrid polymer disclosed herein and an oligonucleotide.

In one or more embodiments, the polyplex transfection complex further includes a hydrophobic polymer polymerized from the same quinoline-containing monomer of the hybrid polymer and a second monomer.

In yet another aspect, the present disclosure describes a method of transfecting a cell. The method includes contacting a cell with a composition of the present disclosure.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing or photograph executed in color. Copies of this patent or patent application publication with color drawing(s) or photographs(s) will be provided by the Office upon request and payment of the necessary fee.

FIG. 1 shows a synthetic scheme for the synthesis of a hydroquinine monomer (HQ) and copolymerization of HQ with 2-hydroxyethyl acrylate (HEA) using the controlled polymerization method of reversible addition fragmentation chain transfer (RAFT). Polymers made via this scheme are referred to as HQ-X polymers, where X indicates the mol-% of the HQ monomer in the polymer.

FIG. 2 shows a synthetic scheme and the chemical structure of a PEG-containing hybrid polymer synthesized from 2-hydroxyethyl acrylate (HEA) and a hydroquinine monomer (HQ). The resultant polymer is referred to as PHQ.

FIG. 3 is a table summarizing particle diameter sizes (nanometers) of 25 different HQ-X and PHQ composition mixtures.

FIG. 4 is a graphical representation of polyplex diameter size (in nanometers) for various polyplex formulations of HQ-X and PHQ over the course of zero to 2 hours.

FIG. 5 shows the data for the effect of the sequence of addition of the hybrid polymer and the hydrophobic polymer into the transfection mixture on the size of the polyplexes.

FIG. 6 shows data from a green fluorescent protein reporter assay evaluating pDNA delivery performance in the presence of hybrid polymers.

FIG. 7 shows the data for a cell viability assay using a colorimetric assay with CCK-8. The values shown are normalized from 0 to 1. A value of 1 means 100% cell survival.

FIG. 8 is a graphical representation of the data from a study evaluating the extent of polyplex internalization. The analysis was done by transfecting cells using polyplexes that included a mixture of a hybrid polymer (PHQ), one of five HQ-X polymers, and Cy5-labeled pDNA.

FIG. 9 is a graphical representation of a dye exclusion assay used to study the ability of hybrid polymer (PHQ) and HQ-X polymer mixtures to bind with pDNA.

FIG. 10 is a synthetic scheme and chemical structure of PEG-CTA prepared by using mPEG-OH and CDP (4-cyano-4-(phenylcarbonothioylthio)pentanoic acid) chain transfer agent (CTA).

FIG. 11 is a plot of the percentage of Cy5 positive cells after treating cells with polyplexes formed with HQ-25 or HQ-35 and various amounts of PHQ.

FIG. 12 is a plot of the percent of GFP positive live cells after cells were treated with polyplexes having different polymer compositions.

FIG. 13 are representative images of the plot in FIG. 12.

FIG. 14 and FIG. 15 show the polyplex diameter, transfection efficiency percent, and cell viability of various polyplex compositions and method of forming such compositions.

FIG. 16 is an illustration showing the three polyplex systems tested. Mixtures using carbohydrate diblock stabilizers give excellent mRNA delivery properties.

FIG. 17 is a synthetic scheme for the synthesis a polymer having hydrophobic block polymerized from 2-hydroxyethyl acrylate (HEA) and a hydroquinine monomer (HQ) and a hydrophilic block polymerized from a carbohydrate-containing monomer. The resultant polymer is termed GlcHQ.

FIG. 18 is a synthetic scheme for the synthesis of a PEG-containing hybrid polymer synthesized from 2-hydroxyethyl acrylate (HEA) and a hydroquinine monomer (HQ). The resultant polymer is referred to as PHQ.

FIG. 19 is a table of the chemical properties of various polymers. ^{a}These polymers were previously synthesized and characterized, see reference Roy, P.; Kreofsky, N. W.; Brown, M. E.; Van Bruggen, C.; Reineke, T. M. Enhancing PDNA Delivery with Hydroquinine Polymers by Modulating Structure and Composition. JACS Au 2023, 3, 1876-1889. ^{b}In the HQ-containing block of the polymer. ^{c}Measured by ¹H NMR. ^{d}Measured by SEC. ^{e}Given some micellization/aggregation of the polyplexes in the aqueous SEC mobile phase, molecular weight was determined using ¹H NMR.

FIGS. 20, 21, 22, 23, 24, and 25 show the size and distribution information for the library of mixtures (n≥5) formulated in Opti-MEM at N/P=16. Stabilizers from top to bottom: GlcHQ (blue), GalHQ (red), PHQ (green). Blank gray boxes indicate colloidally unstable formulations with sizes>1 μm and multimodal distributions. NT stands for not tested. FIGS. 20, 21, and 22 show the hydrodynamic diameters (nm) of the library of formulations showing that increasing the stabilizer percentage decreases the particle size. FIGS. 23, 24, and 25 show the PDI values for the library of formulations showing that increasing the percentage of HQ in the HQ-X component increases the particle size distribution.

FIGS. 26, 27, and 28 show the particle size over time in Opti-MEM (N/P=16) measured via DLS for various polyplex formulations. Generally, all the particle formulations maintain their size after one week of storage at 4° C. Error bars represent standard deviation of the DLS acquisitions (n≥5).

FIG. 29 shows the results of an mRNA binding study for the library of particle formulations. RiboGreen dye exclusion assay showing the change in fluorescence intensity normalized to an mRNA only control upon addition of Opti-MEM for the 8 polymer components in this study. A smaller change in the fluorescence intensity indicates tighter binding to the payload, as the addition of salts and neutralizing the pH does not allow the RiboGreen dye to bind to mRNA in place of polymer and fluoresce. Increasing the HQ percentage leads to tighter binding. Error bars represent standard deviation (n=3).

FIG. 30 shows the results of an mRNA binding study for the library of particle formulations. Gel electrophoresis assay indicates whether the payload is fully compacted and tightly bound by the formulation (no mRNA band) or if there is a fraction of payload that is unbound (mRNA band seen). Wells are labeled with the fraction of HQ in HQ-X followed by the percentage of stabilizer (i.e. 25/5 indicates a formulation of HQ-25 with 5% stabilizer). Increasing the fraction of stabilizer decreases binding strength.

FIG. 31 is an illustration of a fluorescence resonance energy transfer (FRET) assay. The illustration showing the two controls run for each formulation (Cy3 only negative control and co-formulated polyplex positive control) along with the mixed polyplex test condition. After the incubation window, samples were measured to compare the Cy3 emission at 570 nm to determine whether FRET was occurring between the Cy3-Cy5 FRET pair, which would indicate payload exchange.

FIG. 32 are plots showing representative Cy3 emission spectra for a tightly binding formulation, GlcHQ 35/5 (top), and a weakly binding formulation, GlcHQ 35/50 (bottom).

FIG. 33 shows bar graphs representing the fluorescence intensity at the emission maximum (570 nm) of Cy3 for the two controls and the mixed polyplex test condition for GlcHQ 35/5 (top) and GlcHQ 35/50 (bottom). A statistical difference between the negative Cy3 only control and the mixed polyplex formulation indicates FRET has occurred as a result of payload exchange. Error bars represent the range (n=2) and an unpaired t test was used to determine statistical significance (ns=p≥0.05, *=0.05≥p≥0.01, **=0.01≥p≥0.001, ***=p<0.001).

FIG. 34 is a table summarizing of FRET and gel electrophoresis assay data. ^{a} Mixture codes are the HQ content in HQ-X followed by the stabilizer percentage. ^{b} Significance refers to whether there was a significant difference in the Cy3 emission between the Cy3 only negative control and the mixed polyplex test formulation using an unpaired t-test (significance indicated by p<0.05). A Y in this column indicates payload exchange while N denotes no payload exchange was observed. ° Formulations denoted with “B” in the Gel column indicate borderline, neither a definitive Y nor N. The free mRNA bands are extremely faint in these samples.

FIG. 35 and FIG. 36 are heat maps for transfection efficiency (% live cells GFP+) measured by flow cytometry (FIG. 35) and relative cell viability (FIG. 36) measured by a CCK-8 assay for all the formulations that had colloidally stable sizes under 1 μm. The numbers to the right of the HQ-X label are the N/P percentage that comes from the diblock stabilizer. Numbers in heat map are the average of three replicates.

FIG. 37 and FIG. 38 are heat maps for the internalization efficiency (% live cells Cy5+) (FIG. 37) and the Cy5 geometric mean fluorescence intensity (FIG. 38). The former gives information on what percentage of cells have at least one copy of the fluorescently labeled mRNA, while the latter gives information on how much of labeled mRNA was internalized at a populational level (higher MFI=more payload in the cells). The numbers to the right of the HQ-X label are the N/P percentage that comes from the diblock stabilizer. Numbers in heat map are the average of three replicates. Note that the Cy5 geometric MFI heat map is on a logarithmic scale.

FIGS. 39, 40, 41, and 42 are plots showing the transfection efficiencies for the best performing formulations in various cell lines. U=Untreated, N=Naked, J=jetPEI, L=LPF 2000. Treatments are labeled with the HQ-X component followed by percent stabilizer. Across the board, the sugar-based stabilizers perform better than the PEG-based stabilizer in terms of transfection efficiency. In A549 and HuH-7 cells the GalHQ formulations performed better than the GlcHQ (FIGS. 39 and 40); however, this is not seen in the other two cell lines (FIGS. 41 and 42). Error bars represent standard deviation (n=3). Statistical significance is shown between the two sugars (GlcHQ and GalHQ) as well as between the best performing sugar and PHQ for each formulation. Statistical significance or lack thereof was determined using one-way ANOVA followed by post-hoc Tukey analysis for each formulation. p≥0.05=ns, 0.05≥p≥0.01=0.01≥p≥0.0001=**, p<0.0001=***.

FIG. 43 illustrates the three different mixing sequences were performed to understand if the size of the final polyplexes is influenced by the order of polymer addition to pDNA. In mixing method A, both polymers were added to pDNA at the same time. For mixing method B, HQ-X was added first to pDNA and PHQ was added 30 minutes later. In the case of mixing method C, PHQ was added first to pDNA, followed by addition of HQ-X, 30 minutes later.

FIG. 44 is a summary of dynamic light scattering measurements on total 70 polyplex formulations made by using combination of 5 HQ-X polymers with PHQ, in 4 different PHQ incorporation levels, in 3 different mixing methods.

FIGS. 45, 46, and 47 are summary tables of transfection studies using polymer mixtures that result in colloidally stable polyplexes. FIG. 45 shows the transfection efficiency of polymer mixtures in terms of percentage of GFP+ live cells. FIG. 46 shows the normalized viability of the cells after transfection using polymer mixtures. The viability values were normalized to untreated cells that were not treated with any polymers. FIG. 47 shows the effective efficiency of transfection. Effective efficiency is the product of transfection efficiency and normalized cell viability, and it illustrates a whole view of performance by the pDNA carriers. For comparison, polyplexes formed with only the HQ-X component as well as only PHQ were also evaluated. While pure HQ-X polyplexes showed high transfection efficiency and low viability, the polymer mixtures generally showed retention of transfection efficiency and improvement in cell viability. Pure PHQ polyplexes performed not as well with no GFP+ cells. Along those lines, polymer formulations with 50% PHQ incorporation also had low transfection efficiency. All transfections were accompanied by cells transfected with jetPEI, a commercially available transfection agent.

### DETAILED DESCRIPTION OF ILLUSTRATIVE EMBODIMENTS

This disclosure describes hybrid polymers having a hydrophilic block and a hydrophobic block. The hydrophobic block is a quinoline-containing copolymer block. The hydrophilic block can be a polyethylene glycol block or a carbohydrate-containing block.

The hybrid polymers of this disclosure may be useful for delivery genetic material into a cell. For example, the hybrid polymers can be used alone or mixed with a hydrophobic quinoline-containing copolymer in a polyplex transfection complex.

Delivery of an oligonucleotide to the intracellular space of a cell is challenging both in research settings and clinical settings. With the rise in gene editing technology and the potential of gene therapy, there is a need for an improved method of delivering oligonucleotides to cells. Clinical trials have been developed around recombinantly engineered viruses as the delivery vehicle or vector. Viral vectors, however, may have limitations to their application, such as, for example limitations in scaling production, clinical dangers such as immunogenicity and mutagenicity, and/or high cost.

Polymer-based and/or lipid-based oligonucleotide delivery vehicles have emerged as promising alternatives to viral delivery vehicles due to their facile scalability, ease of manufacturing, and thermal stability. For example, several vaccines are packaged in lipid-based vehicles. Additionally, several lipid-based and polymer-based transfection reagents are commercialized and commonly used for transfection in vitro, including the cationic lipid-based reagent LIPOFECTAMINE (Invitrogen, Carlsbad, CA) and the cationic polymer (a linear polyethylenimine derivative) JETPEI (Avantor, Radnor, PA). These reagents, however, show limited efficacy (e.g., limited gene expression and/or editing) in many difficult to transfect cell types. They also can be toxic to target cells, which can be problematic for sensitive and/or useful cell types for clinical applications.

Quinoline-containing polymers can be used to deliver nucleic acids into cells. For example, quinine, a naturally occurring alkaloid renowned for its antimalarial properties and distinctive chemical interactions, is a molecule of significant interest in medicinal and materials chemistry. The structural features of quinoline, including an alkene group, a quinoline ring, a hydroxyl group, and a tertiary amine, contribute to its versatility and effectiveness in various applications.

The quinoline ring influences quinine's stability and binding capabilities through pi-pi interactions, enhancing its binding affinity to biological targets and improving its efficacy as a medicinal agent. The hydroxyl group in quinine participates in hydrogen bonding, influencing solubility and binding. For example, the hydroxyl group enhances solubility in aqueous environments, improving bioavailability and transport within the body. Additionally, the hydroxyl group contributes to the specificity and strength of interactions with biological molecules via hydrogen bonding. The tertiary amine moiety engages in electrostatic interactions, contributing to its binding specificity and biological potency. The alkene group in quinine enables it to participate in addition reactions, presenting potential as a monomer for copolymer synthesis. This attribute is particularly useful in developing polymeric materials for applications such as controlled drug release systems, expanding the scope of quinine's utility beyond traditional medicinal roles.

Quinine has several properties that make it an attractive group to include in a polymer transfection agent. For example, quinine is an FDA-approved antimalarial drug, inexpensive, and naturally sourced. Quinine also has well-characterized intrinsic fluorescent properties, allowing polymers that include quinine to be tracked without using extra dyes. For example, cells that have taken up the polymer may be traced via fluorescence. Additionally, since the fluorescence of quinine is sensitive to pH and chloride ion concentrations, the polymer may be used as a probe for tracking intracellular conditions. Furthermore, quinine is an endosomolytic agent that promotes the endosomolysis of lysosomes, which may allow for the endosomal escape of the oligonucleotide cargo from the endosome. However, quinine-containing polymers are hydrophobic, and hence, their polyplexes may be less colloidally stable. For example, complexes formed that include the binding of quinine-containing polymers and nucleic acids, such as mRNA and pDNA, may aggregate resulting in large aggregates (diameter≥1000 nanometers). For the success of quinine-containing polymers in delivering nucleic acids in more challenging scenarios, such as animal studies or human patients, a particle size of less than 200 nanometers in diameter is often desirable. For example, particles having a size for 200 nm or greater are known to be rapidly cleared and sequestered in the spleen and liver by the reticuloendothelial system.

Incorporating hydrophilic blocks in polymers may be used to deter particles from aggregating. Hydrophilic blocks may be chemically attached to polymer chains. However, to adjust the hydrophilic block content per polymer, a new polymer is synthesized each time.

The present disclosure includes polymers. Polymers can be described by the monomers used to make them. As such, when a polymer is described as including a particular monomer or a repeating unit derived from a particular monomer, the polymer includes the reaction product of the monomer with another monomer of the same kind or different kind. For example, when a monomer includes an alkene polymerizable group, upon polymerization the alkene becomes saturated. For example, polymerization of a —CHCH₂ polymerizable group results in a —CHCH— group where “-” represents a point of connection bond.

Polymers polymerized from a particular monomer may be described as “monomer name” polymer or poly(“monomer name”). For example, a polymer polymerized from a quinoline-containing monomer of the general formula X may be described as an X polymer or poly(X). Similarly, a copolymer polymerized from a quinoline-containing monomer of the general formula X and an acrylamide monomer may be referred to as an X-acrylamide polymer or poly(X-arylamide). The statement “polymerized from” is open-ended and does not limit the polymer being described to being formed solely from the monomer described following the statement. Such polymers may include other monomers. For example, a polymer polymerized from a quinoline-containing monomer may be formed from only the quinoline-containing monomer or may be formed from the quinoline-containing monomer and one or more additional monomers not containing quinoline.

Polymerization of monomers results in repeat units. For example, polymerization of a quinoline-containing monomer with other quinoline-containing monomers and/or other non-quinoline-containing monomers results in a polymer having quinoline-containing repeat units. It is understood that the repeat units of a polymer are derived from the monomers used during polymerization.

In one or more embodiments, a polymer of the present disclosure is a homopolymer or includes a homopolymer block. A homopolymer or a homopolymer block is a polymer polymerized from a single monomer. A homopolymer or a homopolymer block has a single repeat unit. A homopolymer or homopolymer block formed from a single monomer can be functionalized post-polymerization to change the molecular composition of the repeating groups. In one or more embodiments, a polymer of the present disclosure is a copolymer. Copolymers are polymerized from two or more distinct monomers. Polymers made from two or more monomers (e.g., copolymers) may be random polymers, block polymers, graft polymers, or branched polymers.

In one or more embodiments, a polymer of the present disclosure is a block polymer. A block polymer is a polymer that includes two or more polymer segments (blocks) joined by a covalent linkage. A block polymer may include two or more homopolymer blocks, two or more copolymer blocks, or one or more homopolymer blocks and one or more copolymer blocks that are joined by a covalent linkage. Block polymers can be synthesized by polymerizing the first block and then polymerizing the second block directly from one of the terminal groups of the first block.

### Hybrid Polymers

In one aspect, the present disclosure describes a hybrid polymer. A hybrid polymer has a hydrophilic block polymerized from a hydrophilic monomer and a hydrophobic block polymerized from at least one quinoline-containing monomer. In one or more embodiments, a polymer of the present disclosure is a block polymer having a homopolymer hydrophilic block and a copolymer hydrophobic block polymerized from a quinoline-containing monomer and a second monomer.

As used herein, the term “quinoline” refers to the compound having the chemical abstract service registry number 91-22-5 and derivatives thereof wherein one or more of the hydrogen atoms of the quinoline are replaced with a bond to a chemical moiety or with a different atom.

A hydrophobic block of the hybrid polymers is polymerized from at least a quinoline-containing monomer. Quinoline-containing monomers of the present disclosure may be derived from quinine (I), quinidine (II), cinchonidine (III), cinchonine (IV), hydroquinine (V), hydroquinidine (VI), hydrocinchonidine (VII), hydrocinchonine (VIII), combinations thereof, or any stereoisomer thereof.

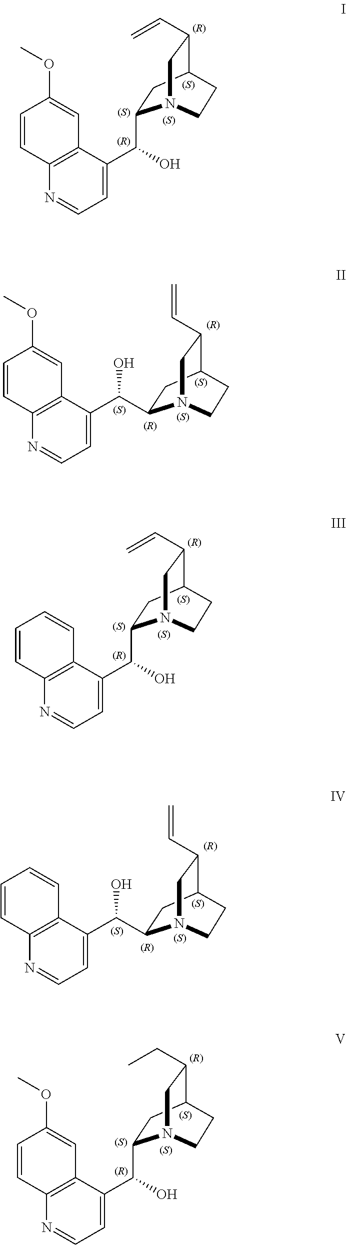

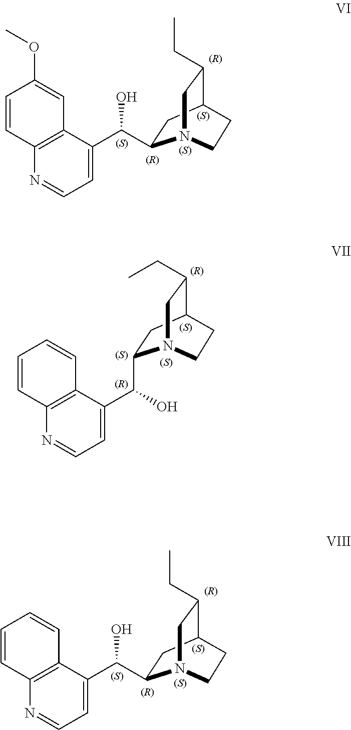

In one or more embodiments, a quinoline-containing monomer is derived from quinine. In one or more embodiments, a quinoline-containing monomer is derived from hydroquinine.

Quinoline-containing monomers may be of the formula:

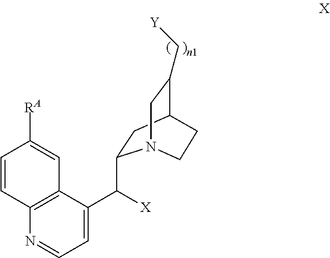

or an ionized form thereof.

In one or more embodiments, a quinoline-containing monomer of the present disclosure is of formula X(a) or X(b):

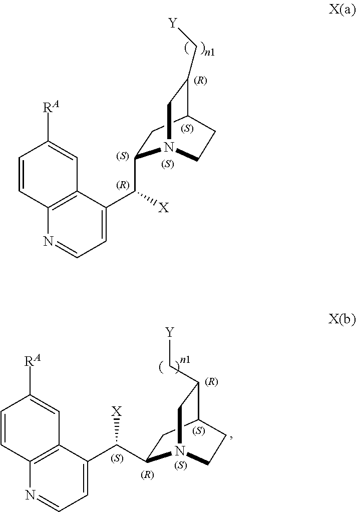

or an ionized form thereof.

In one or more embodiments, a quinoline-containing monomer is of formula X(a). In one or more embodiments, a quinoline-containing monomer is of formula X(b).

In formulas X, X(a), and X(b), R^{A} may be H or an ether of the formula —O—R^{J} where R^{j} is a linear or branch C1 to C10 alkyl. In one or more embodiments, R^{A} is —OCH₃, —OCH₂CH₃, —OCH₂CH₂CH₃, —OCH(CH₃)₂, —OCH₂CH₂CH₂CH₃, —OCH₂CH(CH₂)₃, —OC(CH₃)₃. In one or more embodiments, R^{A} is —OCH₃. In one or more embodiments, R^{A} is H.

In formulas X, X(a), and X(b), n1 is an integer from 1 to 5. In one or more embodiments, n1 is 1. In one or more embodiments, n1 is 2. In one or more embodiments, n1 is 3. In one or more embodiments, n1 is 4. In one or more embodiments, n1 is 5.

In formulas X, X(a), and X(b), X, Y, or both can include a polymerizable group. A polymerizable group is a chemical moiety that is polymerizable either with itself or a different monomer. In one or more embodiments, the polymerizable groups of the present disclosure are alkenes. Examples of polymerizable groups that include alkenes include, but are not limited to, vinyl ethers, vinyl esters, acrylates, acrylamides, methacrylates, and methacrylamides.

In formulas X, X(a), and X(b), X may include a polymerizable group. In formulas X, X(a), and X(b), X may be H, or OH. In one or more embodiments, X is H, OH,

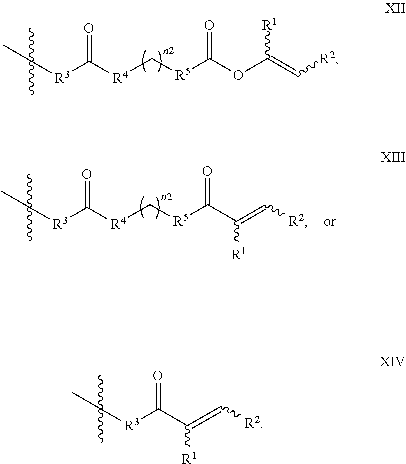

In formulas XII, XIII, and XIV, R³, R⁴, and R⁵ are each independently O, NH, NR²⁰, or S. R²⁰ is alkyl. In one or more embodiments, R²⁰ is methyl, ethyl, n-propyl, or isopropyl. In one or more embodiments, R³ is O. In one or more embodiments, R⁴ is NH. In one or more embodiments, R⁵ is O.

In formulas XII, XIII, and XIV, R¹ and R² are each independently H or methyl (—CH₃). In one or more embodiments, R¹ is H. In one or more embodiments, R² is H. In one or more embodiments, R¹ and R² are H. In one or more embodiments, R² is methyl. In one or more embodiments, R¹ is methyl. In one or more embodiments, R¹ and R² are methyl. In one or more embodiments, R¹ is methyl, and R² is H. In one or more embodiments, R² is methyl, and R¹ is H.

In formulas XII, XIII, and XIV, n2 is zero or an integer from 1 to 5. In one or more embodiments, n2 is 0. In one or more embodiments, n2 is 1. In one or more embodiments, n2 is 2. In one or more embodiments, n2 is 3. In one or more embodiments, n2 is 4. In one or more embodiments, n2 is 5.

In formulas X, X(a), and X(b), X may be a group that does not include a polymerization functional group. In one or more embodiments, X is OH. In one or more embodiments, X is H.

In formulas X, X(a), and X(b), Y may include a polymerizable group. In one or more embodiments, Y may be a group that does not include a polymerizable group. Y may be H, CH₃, alkyl, alkenyl,

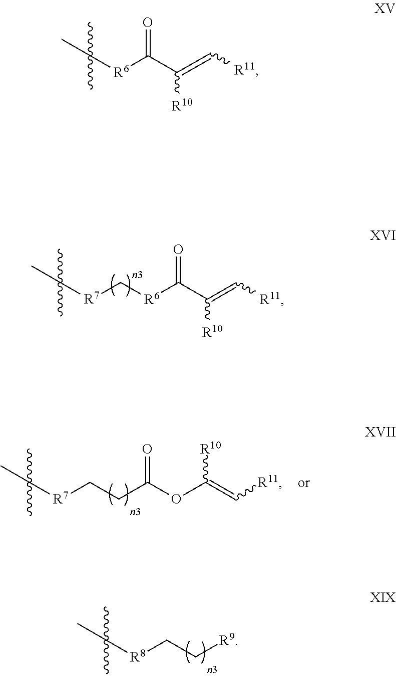

In formulas XV, XVI, XVII, and XIX, R⁶, R⁷, and R⁸ are each independently O, NH, NR²¹, or S. R²¹ is alkyl. In one or more embodiments, R²¹ is methyl, ethyl, n-propyl, or isopropyl. In one or more embodiments, R⁶ is O. In one or more embodiments, R⁶ is NH. In one or more embodiments, R⁷ is O. In one or more embodiments, R⁷ is NH. In one or more embodiments, R⁷ is S.

In formulas XV, XVI, XVII, and XIX, R¹⁰ and R¹¹ are each independently H or methyl. In one or more embodiments, R¹⁰ is H. In one or more embodiments, R¹¹ is H. In one or more embodiments, R¹⁰ and R¹¹ are H. In one or more embodiments, R¹⁰ is methyl. In one or more embodiments, R¹¹ is methyl. In one or more embodiments, R¹⁰ and R¹¹ are methyl. In one or more embodiments, R¹⁰ is methyl, and R¹¹ is H. In one or more embodiments, R¹¹ is methyl, and R¹⁰ is H.

In formulas XVI, XVII, and XIX, n3 is zero or an integer from 1 to 5. In one or more embodiments, n3 is 0. In one or more embodiments, n3 is 1. In one or more embodiments, n3 is 2. In one or more embodiments, n3 is 3. In one or more embodiments, n3 is 4. In one or more embodiments, n3 is 5.

In one or more embodiments of formulas X, X(a), and X(b), Y is an alkyl. In one or more embodiments, Y is a C1, C2, C3, C4, C5, C6, C7, C8, C9, or C10 alkyl. In one or more embodiments, Y is methyl (CH₃), ethyl (CH₂CH₃), propyl (CH₂CH₂CH₃; C(CH₃)₂), or butyl (CH₂CH₂CH₂CH₃; CH(CH₃)CH₂CH₃; CH₂CCH₃)₂). In one or more embodiments, Y is CH₃.

In one or more embodiments of formulas X, X(a), and X(b), Y is alkenyl. In one or more embodiments, Y is a C1, C2, C3, C4, C5, C6, C7, C8, C9, or C10 alkenyl. In one or more embodiments, the alkene of the alkenyl is a terminal alkene. In one or more embodiments, Y is —CHCH₂.

Example of quinoline-containing monomers include

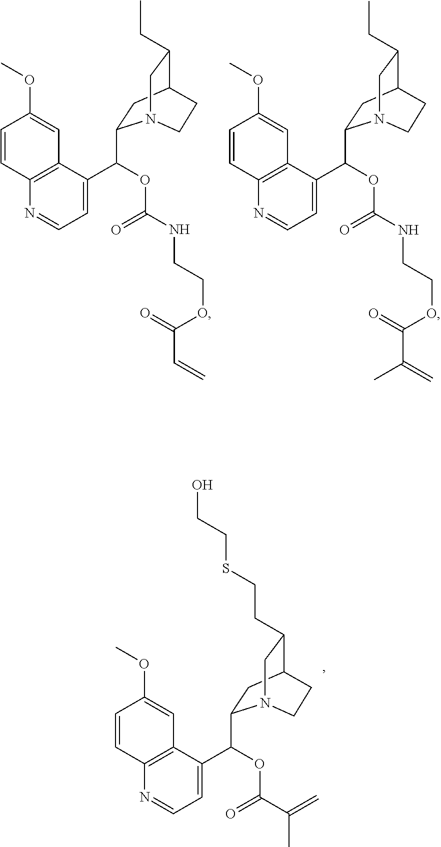

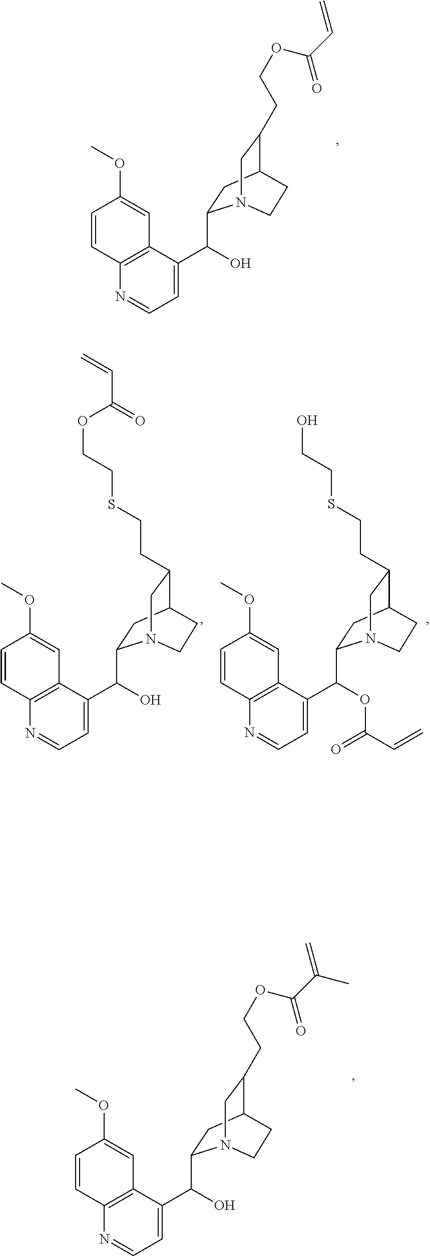

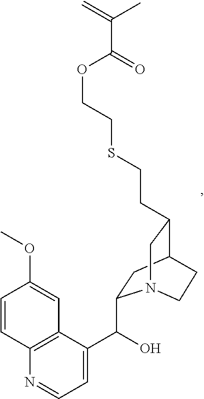 [SMILES: C=C(C)C(=O)OCCSCCC1CN2CCC1CC2C(O)c1ccnc2ccc(OC)cc12]

or an ionized form thereof.

Further examples of quinoline-containing monomers include

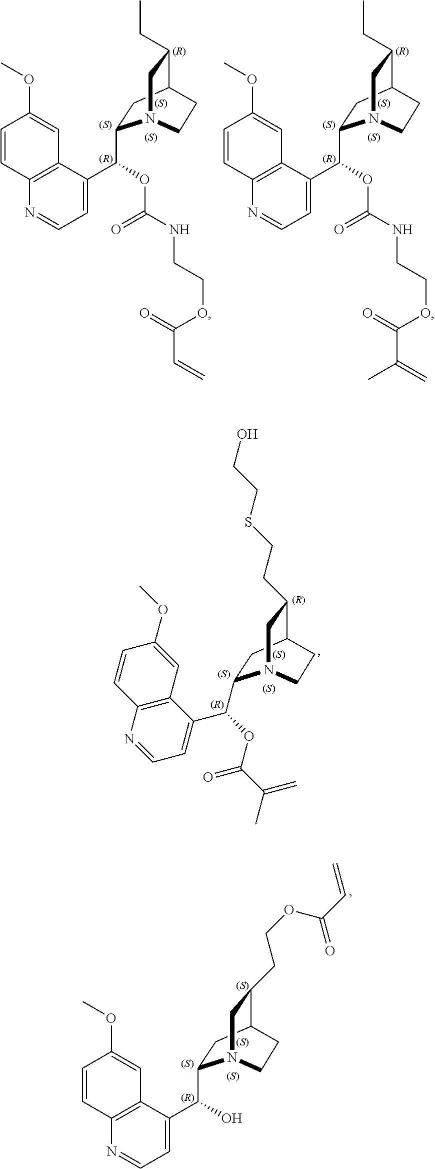

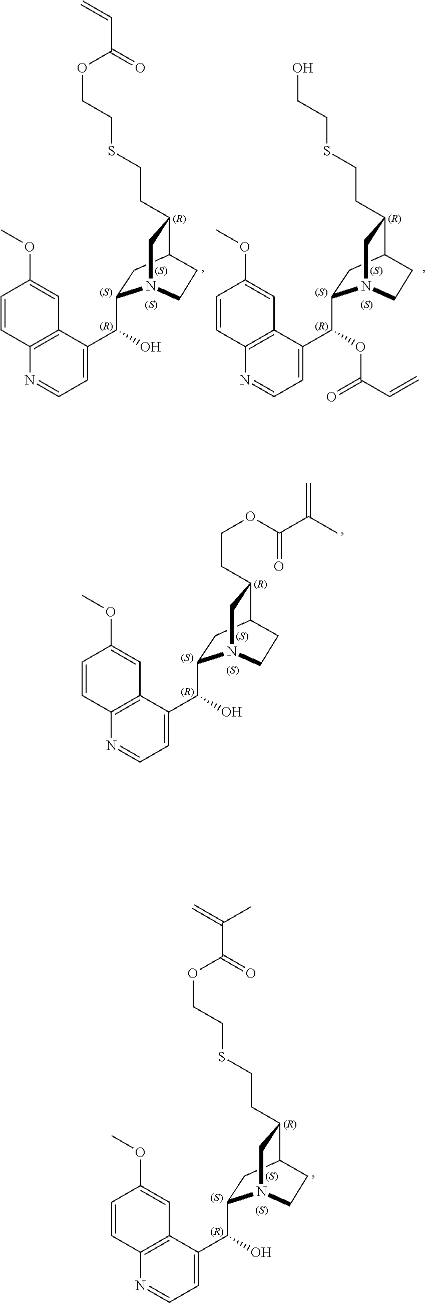

or an ionized from thereof.

Polymerization of a quinoline-containing monomer results in quinoline-containing repeat units. In one or more embodiments, a hybrid polymer includes one or more of the following quinoline-containing repeat units:

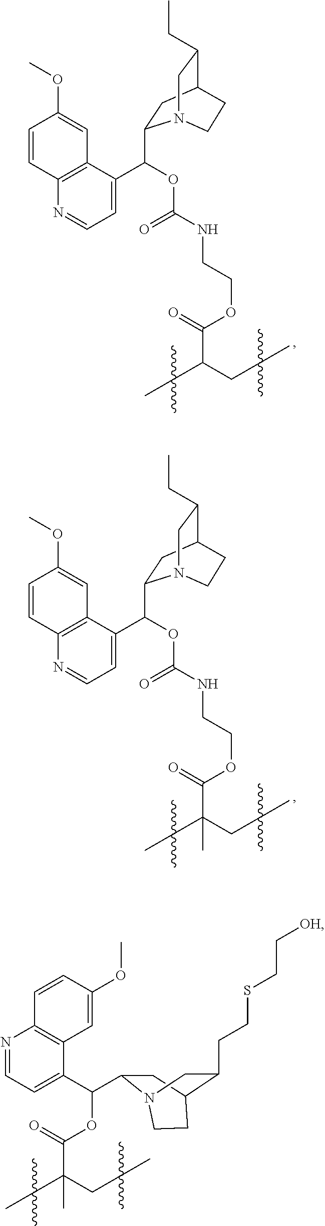

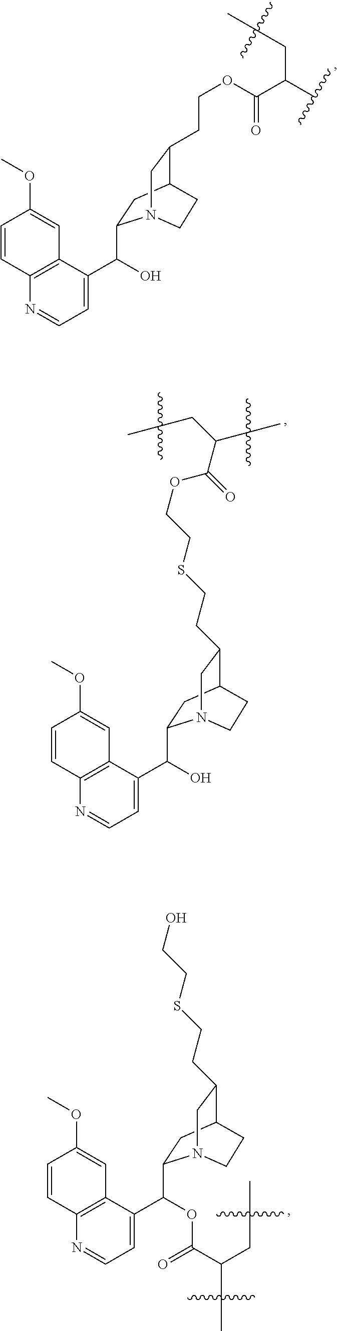

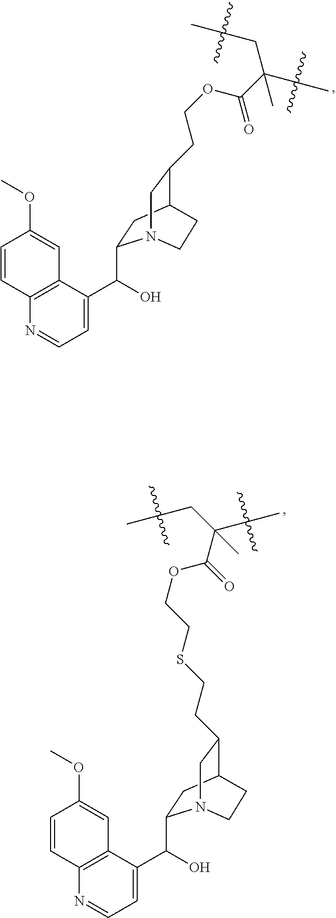

or an ionized form thereof.

In one or more embodiments, a hybrid polymer includes one or more of the following quinoline-containing repeat units:

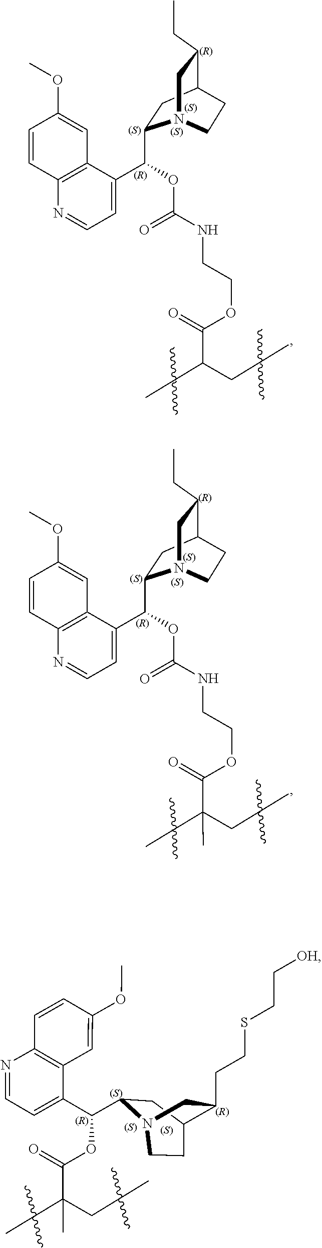

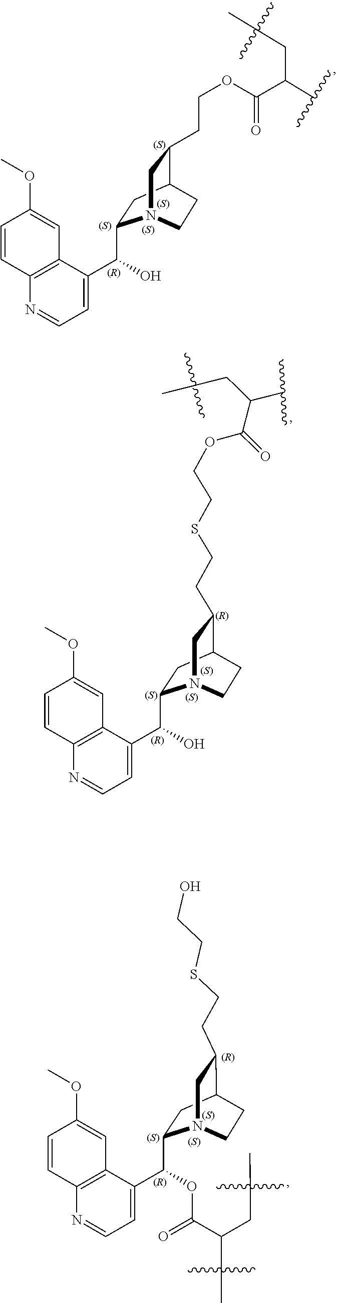

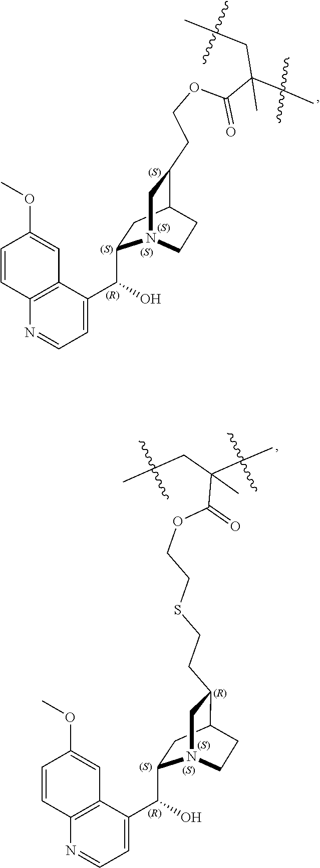

or an ionized form thereof.

In one or more embodiments, a hybrid polymer may include more than one type of quinoline-containing monomer and/or quinoline-containing repeat unit. For example, in one or more embodiments, a hybrid polymer may include one, two, three, four, or five different quinoline-containing monomers and/or repeat units.

In one or more embodiments, a hydrophobic block of hybrid polymer is a copolymer polymerized from a quinoline-containing monomer and a second monomer. A quinoline-containing monomer may be any quinoline-containing monomer described herein.

In one or more embodiments, a copolymer block (e.g., a hydrophobic copolymer block of a hybrid polymer) is an alternating copolymer block. An alternating copolymer, or alternative copolymer block, is a polymer or block where the first monomer and the second monomer (and any other monomer) alternate along the polymer chain.

In one or more embodiments, a copolymer block (e.g., a hydrophobic copolymer block of a hybrid polymer) is a statistical copolymer block. A statistical copolymer, or a statistical copolymer block, is a polymer where the first monomer and the second monomer are arranged in a sequential order that follows a statistical rule. Examples of statistical rules that a copolymer may follow include, but are not limited to, a Bernoullian distribution (random copolymer) or Markovian distribution.

In one or more embodiments, a copolymer block (e.g., a hydrophobic copolymer block of a hybrid polymer) may include additional monomers beyond the first and second monomers. In one or more embodiments, the copolymer may include a third, fourth, or fifth monomer. Any additional monomers may be any of the quinoline-containing monomers as described elsewhere herein. Any additional monomers may be acrylates, methacrylates, acrylamides, methacrylamides, vinyl monomers, quinoline-containing monomers, or combinations thereof.

In one or more embodiments, a hybrid polymer of the present disclosure includes a hydrophobic block polymerized from a quinoline-containing monomer and one or more additional monomers. The amount of the quinoline-containing monomer/quinoline-containing repeat units in the hydrophobic block of the hybrid polymers of the present disclosure may vary. The amount of the quinoline-containing monomer in polymer may affect the properties of the polymer. The mole percent (mol-%) of the quinoline-containing monomer/quinoline-containing repeat units in a hybrid polymer or block of a hybrid polymer may be measured and calculated using ¹H NMR via methods known in the art. In one or more embodiments, the amount of a quinoline-containing monomer/quinoline-containing repeat units in a hybrid polymer or hydrophobic block of a hybrid polymer is 1 mol-% or greater, 5 mol-% or greater, 10 mol-% or greater, 15 mol-% or greater, 20 mol-% or greater, 25 mol-% or greater, 30 mol-% or greater, 60 mol-% or greater, 80 mol-% or greater. In one or more embodiments, the amount of a quinoline-containing monomer/quinoline-containing repeat units in a hybrid polymer or hydrophobic block of a hybrid polymer is 100 mol-% or less, 80 mol-% or less, 60 mol-% or less, 30 mol-% or less, 25 mol-% or less, 20 mol-% or less, or 15 mol-% or less, 10 mol-% or less, or 5 mol-% or less. In one or more embodiments, the amount of a quinoline-containing monomer/quinoline-containing repeat units in a hybrid polymer or hydrophobic block of a hybrid polymer is 1 mol-% to 100 mol-%, 1 mol-% to 80 mol-%, 1 mol-% to 60 mol-%, 1 mol-% to 30 mol-%, 1 mol-% to 25 mol-%, 1 mol-% to 20 mol-%, 1 mol-% to 15 mol-%, 1 mol-% to 10 mol-%, or 1 mol-% to 5 mol-%. In one or more embodiments, the amount of a quinoline-containing monomer/quinoline-containing repeat units in a hybrid polymer or hydrophobic block of a hybrid polymer is 5 mol-% to 100 mol-%, 5 mol-% to 80 mol-%, 5 mol-% to 60 mol-%, 5 mol-% to 30 mol-%, 5 mol-% to 25 mol-%, 5 mol-% to 20 mol-%, 5 mol-% to 15 mol-%, or 5 mol-% to 10 mol-%. In one or more embodiments, the amount of a quinoline-containing monomer/quinoline-containing repeat units in a hybrid polymer or hydrophobic block of a hybrid polymer is 10 mol-% to 100 mol-%, 10 mol-% to 80 mol-%, 10 mol-% to 60 mol-%, 10 mol-% to 30 mol-%, 10 mol-% to 25 mol-%, 10 mol-% to 20 mol-%, or 10 mol-% to 15 mol-%. In one or more embodiments, the amount of a quinoline-containing monomer/quinoline-containing repeat units in a hybrid polymer or hydrophobic block of a hybrid polymer is 15 mol-% to 100 mol-%, 15 mol-% to 80 mol-%, 15 mol-% to 60 mol-%, 15 mol-% to 30 mol-%, 15 mol-% to 25 mol-%, or 15 mol-% to 20 mol-%. In one or more embodiments, the amount of a quinoline-containing monomer/quinoline-containing repeat units in a hybrid polymer or hydrophobic block of a hybrid polymer is 20 mol-% to 100 mol-%, 20 mol-% to 80 mol-%, 20 mol-% to 60 mol-%, 20 mol-% to 30 mol-%, or 20 mol-% to 25 mol-%. In one or more embodiments, the amount of a quinoline-containing monomer/quinoline-containing repeat units in a hybrid polymer or hydrophobic block of a hybrid polymer is 25 mol-% to 100 mol-%, 25 mol-% to 80 mol-%, 25 mol-% to 60 mol-%, or 25 mol-% to 30 mol-%. In one or more embodiments, the amount of a quinoline-containing monomer/quinoline-containing repeat units in a hybrid polymer or hydrophobic block of a hybrid polymer is 30 mol-% to 100 mol-%, 30 mol-% to 80 mol-%, or 30 mol-% to 60 mol-%. In one or more embodiments, the amount of a quinoline-containing monomer/quinoline-containing repeat units in a hybrid polymer or hydrophobic block of a hybrid polymer is 60 mol-% to 100 mol-% or 60 mol-% to 80 mol-%. In one or more embodiments, the amount of a quinoline-containing monomer/quinoline-containing repeat units in a hybrid polymer or hydrophobic block of a hybrid polymer is 80 mol-% to 100 mol-%. In one or more embodiments, the amount of a quinoline-containing monomer/quinoline-containing repeat units in a hybrid polymer or hydrophobic block of a hybrid polymer is 15 mol-% to 60 mol-%. In one or more embodiments, the amount of a quinoline-containing monomer/quinoline-containing repeat units in a hybrid polymer or hydrophobic block of a hybrid polymer is 15 mol-% to 30 mol-%.

The second monomer in a hydrophobic block of a hybrid polymer of the present disclosure may be any monomer suitable for polymerization. In one or more embodiments, the second monomer includes an alkene polymerizable group. In one or more embodiments, the second monomer is an acrylate, methacrylate, acrylamide, methacrylamide, or a vinyl monomer.

In one or more embodiments, the second monomer is an acrylate. Examples of acrylate monomers include but are not limited to, benzyl acrylate; methyl acrylate; ethyl acrylate; n-propyl acrylate; isopropyl acrylate; iso-butyl acrylate; tert-butyl acrylate; sec-butyl acrylate; iso-decyl arylate; heptadecyl acrylate; ethyldiglycol acrylate; 4-hydroxybutyl acrylate; 2-hydroxyethyl acrylate (HEA); 2-ethyl hexyl acrylate; hydroxyethyl caprolactone acrylate; hydroxypropyl acrylate; lauryl acrylate; 2-propyl heptyl acrylate; stearyl acrylate; poly(ethylene glycol) methyl ether acrylate; poly(ethylene glycol) acrylate; 2,3-dihydroxypropyl acrylate; 2-acryloyloxyethyl phosphorylcholine; [2-(acryloyloxy)ethyl]dimethyl-(3-sulfopropyl)ammonium hydroxide; 2-carboxyethyl acrylate, acrylic acid, 2-(dimethylamino)ethyl acrylate; N-acryloyl morpholine; and benzhydryl acrylate. In one or more embodiments, the second monomer is HEA.

In one or more embodiments, the second monomer is a methacrylate. Examples of methacrylate monomers include but are not limited to; benzyl methacrylate; methyl methacrylate; ethyl methacrylate; n-propyl methacrylate; isopropyl methacrylate; iso-butyl methacrylate; tert-butyl methacrylate; sec-butyl methacrylate; iso-decyl methacrylate; heptadecyl methacrylate; ethyldiglycol methacrylate; 4-hydroxybutyl methacrylate; 2-hydroxyethyl methacrylate (HEMA); 2-ethylhexyl methacrylate; 2,3-dihydroxypropyl methacrylate; [2-(methacryloyloxy)ethyl]dimethyl-(3-sulfopropyl)ammonium hydroxide; 2-methacryloyloxyethyl phosphorylcholine; 2-carboxyethyl methacrylate; glycidyl methacrylate; 2-(dimethylamino)ethyl methacrylate; 2-aminoethyl methacrylate; hydroxyethyl caprolactone methacrylate; hydroxypropyl methacrylate; lauryl methacrylate; 2-propyl heptyl methacrylate; stearyl methacrylate; N-methacryloyl morpholine; benzhydryl methacrylate; poly(ethylene glycol) methyl ether methacrylate; poly(ethylene glycol) methacrylate; and 2-n-butoxyethyl methacrylate. In one or more embodiments, the second monomer is methyl methacrylate.

In one or more embodiments, the second monomer is an acrylamide. Examples of acrylamide monomers include, but are not limited to, acrylamide; N,N-dimethyl acrylamide (DMA); N-isopropyl acrylamide; N-ethyl methyl acrylamide; N-ethyl acrylamide; poly(ethylene glycol) methyl ether acrylamide; poly(ethylene glycol) acrylamide; 6-acrylamidohexanoic acid; 3-[(3-acrylamidopropyl)dimethylammonio]propane-1-sulfonate; 3-[(3-acrylamidopropyl)dimethylammonio]propanoate; (3-acrylamidopropyl)trimethylammonium chloride; 2-aminoethylmethacrylamide; and (2-hydroxyethyl)acrylamide. In one or more embodiments, the second monomer is (2-hydroxyethyl)acrylamide. In one or more embodiments, the second monomer is N-isopropyl acrylamide. In one or more embodiments, the second monomer is N,N-dimethyl acrylamide.

In one or more embodiments, the second monomer is a methacrylamide. Examples of methacrylamide monomers include, but are not limited to, methacrylamide, N,N-dimethyl methacrylamide, N-isopropyl methacrylamide, N-isopropyl methyl methacrylamide, N,N-dimethyl methacrylamide, N-ethyl methyl methacrylamide, N-ethyl methacrylamide, poly(ethylene glycol) methyl ether methacrylamide, poly(ethylene glycol) methacrylamide, and (2-hydroxyethyl)methacrylamide. In one or more embodiments, the second monomer is (2-hydroxyethyl)methacrylamide. In one or more embodiments, the second monomer is N-isopropyl methacrylamide. In one or more embodiments, the second monomer is N,N-dimethyl methacrylamide.

In one or more embodiments, the second monomer is a vinyl monomer. In one or more embodiments, the vinyl monomer is a vinyl ester, vinyl amide, or vinyl ether. Examples of vinyl monomers include but are not limited to, vinyl acetate; allyl acetate; allyl alcohol; 3-allyloxy-1,2-propanediol; vinyl cinnamate; ethyl vinyl ether; acrylonitrile; ethylene; propylene; styrene; vinyl chloride; vinyl propionate; vinyl laurate; ethyl vinyl ether; iso-butyl vinyl ether; cyclohexyl vinyl ether; dodecyl vinyl ether; octadecyl vinyl ether; hydroxyl butyl vinyl ether; 3-aminopropyl vinyl ether; N-vinyl-N-methyl acetamide; N-vinyl imidazole; N-vinyl pyrrolidone; allyl glycidyl ether; 1,2-epoxy-5-hexene; 3,4-epoxy-i-butene; allylamine; and vinyl methyl oxazolidinone. In one or more embodiments, the second monomer is vinyl acetate.

The number-average molecular weight (Mn) of the hybrid polymers and/or a hydrophobic block of the present application may vary. Mn is calculated using the following equation:

$$Mn = \sum {x}_{i} {M}_{i}$$

where Mᵢ is the mean molecular size of range i and xᵢ is the number fraction of the total number of copolymer chains that are within the Mᵢ range. Mn may be determined using size exclusion chromatography with a multi-angle light scattering detector (see Example).

In one or more embodiments, the Mn of a hybrid polymer and/or a hydrophobic block of a hybrid polymer is 5 kilodaltons (kDa) or greater, 10 kDa or greater, 20 kDa or greater, 25 kDa or greater, 30 kDa or greater, 40 kDa or greater, 50 kDa or greater, 75 kDa or greater, 100 kDa or greater, 125 kDa or greater, 150 kDa or greater, 175 kDa or greater, 200 kDa or greater, 225 kDa or greater, 250 kDa or greater, or 275 kDa or greater. In one or more embodiments, the Mn of a hybrid polymer and/or a hydrophobic block of a hybrid polymer is 300 kDa or less, 275 kDa or less, 250 kDa or less, 225 kDa or less, 200 kDa or less, 175 kDa or less, 150 kDa or less, 125 kDa or less, 100 kDa or less, 75 kDa or less, 50 kDa or less, 40 kDa or less, 30 kDa or less, 25 kDa or less, 20 kDa or less, or 10 kDa or less. In one or more embodiments, the Mn of a hybrid polymer and/or a hydrophobic block of a hybrid polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mn of a hybrid polymer and/or a hydrophobic block of a hybrid polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mn of a hybrid polymer and/or a hydrophobic block of a hybrid polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mn of a hybrid polymer and/or a hydrophobic block of a hybrid polymer is 25 kDa to 100 kDa, 25 kDa to 75 kDa, 25 kDa to 50 kDa, 25 kDa to 40 kDa, or 25 kDa to 30 kDa. In one or more embodiments, the Mn of a hybrid polymer and/or a hydrophobic block of a hybrid polymer is 30 kDa to 100 kDa, 30 kDa to 75 kDa, 30 kDa to 50 kDa, or 30 kDa to 40 kDa. In one or more embodiments, the Mn of a hybrid polymer and/or a hydrophobic block of a hybrid polymer is 40 kDa to 100 kDa, 40 kDa to 75 kDa, or 40 kDa to 50 kDa. In one or more embodiments, the Mn of a hybrid polymer and/or a hydrophobic block of a hybrid polymer is 50 kDa to 100 kDa or 50 kDa to 75 kDa. In one or more embodiments, the Mn of a hybrid polymer and/or a hydrophobic block of a hybrid polymer is 75 kDa to 100 kDa.

The weight-average molecular weight (Mw) of a hybrid polymer and/or a hydrophobic block of a hybrid polymer of the present application may vary. Mw is calculated using the following equation:

$$Mw = \sum {w}_{i} {M}_{i}$$

where Mᵢ is the mean molecular size of range i and wᵢ is the weight fraction of the total number of copolymer chains that are within the Mᵢ range. Mw may be determined using size exclusion chromatography with a multi-angle light scattering detector (see Example).

In one or more embodiments, the Mw of hybrid polymer and/or a hydrophobic block of a hybrid polymer is 5 kilodaltons (kDa) or greater, 10 kDa or greater, 20 kDa or greater, 25 kDa or greater, 30 kDa or greater, 40 kDa or greater, 50 kDa or greater, 75 kDa or greater, 100 kDa or greater, 125 kDa or greater, 150 kDa or greater, 175 kDa or greater, 200 kDa or greater, 225 kDa or greater, 250 kDa or greater, or 275 kDa or greater. In one or more embodiments, the Mw of hybrid polymer and/or a hydrophobic block of a hybrid polymer is 300 kDa or less, 275 kDa or less, 250 kDa or less, 225 kDa or less, 200 kDa or less, 175 kDa or less, 150 kDa or less, 125 kDa or less, 100 kDa or less, 75 kDa or less, 50 kDa or less, 40 kDa or less, 30 kDa or less, 25 kDa or less, 20 kDa or less, or 10 kDa or less. In one or more embodiments, the Mw of hybrid polymer and/or a hydrophobic block of a hybrid polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mw of hybrid polymer and/or a hydrophobic block of a hybrid polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mw of hybrid polymer and/or a hydrophobic block of a hybrid polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mw of hybrid polymer and/or a hydrophobic block of a hybrid polymer is 25 kDa to 100 kDa, 25 kDa to 75 kDa, 25 kDa to 50 kDa, 25 kDa to 40 kDa, or 25 kDa to 30 kDa. In one or more embodiments, the Mw of hybrid polymer and/or a hydrophobic block of a hybrid polymer is 30 kDa to 100 kDa, 30 kDa to 75 kDa, 30 kDa to 50 kDa, or 30 kDa to 40 kDa. In one or more embodiments, the Mw of hybrid polymer and/or a hydrophobic block of a hybrid polymer is 40 kDa to 100 kDa, 40 kDa to 75 kDa, or 40 kDa to 50 kDa. In one or more embodiments, the Mw of a hybrid polymer and/or a hydrophobic block of a hybrid polymer is 50 kDa to 100 kDa, or 50 kDa to 75 kDa. In one or more embodiments, the Mw of a hybrid polymer and/or a hydrophobic block of a hybrid polymer is 75 kDa to 100 kDa.

The dispersity of the molecular weight of a hybrid polymer and/or a hydrophobic block of a hybrid polymer affects the characteristics of the polymer. The molecular weight dispersity may be quantified as the dispersity (Ð). Ð is the distribution of individual molecular masses of a polymer. Ð is calculated as the quotient of the mass average molecular weight (M_{w}) divided by the number-average molecular weight (Mₙ). The M_{w} and Mₙ may be determined using various methods, including viscometry, size exclusion chromatography, and mass spectrometry. Generally, a small Ð is preferred. Although there is no desired lower limit, in practice, the Ð of a hybrid polymer and/or a hydrophobic block of a hybrid polymer may be 1.0 or greater, 1.1 or greater, 1.2 or greater, 1.3 or greater, 1.4 or greater, 1.5 or greater, 1.6 or greater, 1.7 or greater, 1.8 or greater, 1.9 or greater, 2.0 or greater, 2.1 or greater, 2.2 or greater, 2.3 or greater. 2.3 or greater, or 2.4 or greater. In some embodiments, the Ð of hybrid polymer and/or a hydrophobic block of a hybrid polymer may be 2.5 or less, 2.2 or less, 2.0 or less, 1.8 or less, 1.7 or less, 1.6 or less, 1.5 or less, 1.4 or less, 1.3 or less, 1.2 or less, or 1.1 or less. In some embodiments, the Ð for a hybrid polymer and/or a hydrophobic block of a hybrid polymer is 1.0 to 2.5, 1.0 to 2.2, 1.0 to 2.0, 1.0 to 1.8, 1.0 to 1.7, 1.0 to 1.6, 1.0 to 1.5, 1.0 to 1.4, 1.0 to 1.3, 1.0 to 1.2, or 1.0 to 1.1. In some embodiments, the Ð for a hybrid polymer and/or a hydrophobic block of a hybrid polymer is 1.1 to 2.5, 1.1 to 2.2, 1.1 to 2.0, 1.1 to 1.8, 1.1 to 1.7, 1.1 to 1.6, 1.1 to 1.5, 1.1 to 1.4, 1.1 to 1.3, or 1.1 to 1.2. In some embodiments, the Ð for hybrid polymer and/or a hydrophobic block of a hybrid polymer is 1.2 to 2.5, 1.2 to 2.2, 1.2 to 2.0, 1.2 to 1.8, 1.2 to 1.7, 1.2 to 1.6, 1.2 to 1.5, 1.2 to 1.4, or 1.2 to 1.3. In some embodiments, the Ð for a hybrid polymer and/or a hydrophobic block of a hybrid polymer is 1.3 to 2.5, 1.3 to 2.2, 1.3 to 2.0, 1.3 to 1.8, 1.3 to 1.7, 1.3 to 1.6, 1.3 to 1.5, or 1.3 to 1.4. In some embodiments, the Ð for hybrid polymer and/or a hydrophobic block of a hybrid polymer is 1.4 to 2.5, 1.4 to 2.2, 1.4 to 2.0, 1.4 to 1.8, 1.4 to 1.7, 1.4 to 1.6, or 1.4 to 1.5. In some embodiments, the Ð for a hybrid polymer and/or a hydrophobic block of a hybrid polymer is 1.5 to 2.5, 1.5 to 2.2, 1.5 to 2.0, 1.5 to 1.8, 1.5 to 1.7, or 1.5 to 1.6. In some embodiments, the Ð for a hybrid polymer and/or a hydrophobic block of a hybrid polymer is 1.6 to 2.5, 1.6 to 2.2, 1.6 to 2.0, 1.6 to 1.8, or 1.6 to 1.7. In some embodiments, the Ð for hybrid polymer and/or a hydrophobic block of a hybrid polymer is 1.7 to 2.5, 1.7 to 2.2, 1.7 to 2.0, or 1.7 to 1.8. In some embodiments, the Ð for hybrid polymer and/or a hydrophobic block k of a hybrid polymer is 1.8 to 2.5, 1.8 to 2.2, or 1.8 to 2.0. In some embodiments, the Ð for hybrid polymer and/or a hydrophobic block of a hybrid polymer is 2.0 to 2.5, 2.0 to 2.2, or 2.2 to 2.5.

The pKa of a hybrid polymer and/or a hydrophobic block of a hybrid polymer may affect the characteristics of the polymer. For example, the pKa of a polymer may affect the ability of the polymer to form complexes (e.g., polyplexes) with oligonucleotides and/or the ability to release oligonucleotides from a polymer-oligonucleotide complex. The pKa of a polymer may be determined using a potentiometric titration. In one or more embodiments, the pKa of a hybrid polymer and/or a hydrophobic block of a hybrid polymer is 4.0 or greater, 4.5 or greater, 5.0 or greater, 5.5 or greater, 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, 7.0 or greater, 7.2 or greater, or 7.5 or greater. In one or more embodiments, the pKa of hybrid polymer and/or a hydrophobic block of a hybrid polymer is 8.0 or less, 7.5 or less, 7.2 or less, 7.0 or less, 6.9 or less, 6.8 or less, 6.7 or less, 6.6 or less, 6.5 or less, 6.4 or less, 6.3 or less, 6.2 or less, 6.1 or less, 6.0 or less, 5.5 or less, 5.0 or less, or 4.5 or less. In one or more embodiments the pKa of hybrid polymer and/or a hydrophobic block of a hybrid polymer is 5.0 to 8.0, 5.0 to 7.5, 5.0 to 7.2, 5.0 to 7.0, 5.0 to 6.9, 5.0 to 6.8, 5.0 to 6.7, 5.0 to 6.6, 5.0 to 6.5, 5.0 to 6.4, 5.0 to 6.3, 5.0 to 6.2, 5.0 to 6.1, 5.0 to 6.0, or 5.0 to 5.5. In one or more embodiments the pKa of a hybrid polymer and/or a hydrophobic block of a hybrid polymer is 5.5 to 8.0, 5.5 to 7.5, 5.5 to 7.2, 5.5 to 7.0, 5.5 to 6.9, 5.5 to 6.8, 5.5 to 6.7, 5.5 to 6.6, 5.5 to 6.5, 5.5 to 6.4, 5.5 to 6.3, 5.5 to 6.2, 5.5 to 6.1, or 5.5 to 6.0. In one or more embodiments the pKa of a hybrid polymer and/or a hydrophobic block of a hybrid polymer is 6.0 to 8.0, 6.0 to 7.5, 6.0 to 7.2, 6.0 to 7.0, 6.0 to 6.9, 6.0 to 6.8, 6.0 to 6.7, 6.0 to 6.6, 6.0 to 6.5, 6.0 to 6.4, 6.0 to 6.3, 6.0 to 6.2, or 6.0 to 6.1. In one or more embodiments the pKa of a hybrid polymer and/or a hydrophobic block of a hybrid polymer is 6.1 to 8.0, 6.1 to 7.5, 6.1 to 7.2, 6.1 to 7.0, 6.1 to 6.9, 6.1 to 6.8, 6.1 to 6.7, 6.1 to 6.6, 6.1 to 6.5, 6.1 to 6.4, 6.1 to 6.3, or 6.1 to 6.2. In one or more embodiments the pKa of a hybrid polymer and/or a hydrophobic block of a hybrid polymer is 6.2 to 8.0, 6.2 to 7.5, 6.2 to 7.2, 6.2 to 7.0, 6.2 to 6.9, 6.2 to 6.8, 6.2 to 6.7, 6.2 to 6.6, 6.2 to 6.5, 6.2 to 6.4, or 6.2 to 6.3. In one or more embodiments the pKa of hybrid polymer and/or a hydrophobic block of a hybrid polymer is 6.3 to 8.0, 6.3 to 7.5, 6.3 to 7.2, 6.3 to 7.0, 6.3 to 6.9, 6.3 to 6.8, 6.3 to 6.7, 6.3 to 6.6, 6.3 to 6.5, or 6.3 to 6.4. In one or more embodiments the pKa of hybrid polymer and/or a hydrophobic block of a hybrid polymer is 6.4 to 8.0, 6.4 to 7.5, 6.4 to 7.2, 6.4 to 7.0, 6.4 to 6.9, 6.4 to 6.8, 6.4 to 6.7, 6.4 to 6.6, or 6.4 to 6.5. In one or more embodiments the pKa of hybrid polymer and/or a hydrophobic block of a hybrid polymer is 6.5 to 8.0, 6.5 to 7.5, 6.5 to 7.2, 6.5 to 7.0, 6.5 to 6.9, 6.5 to 6.8, 6.5 to 6.7, or 6.5 to 6.6. In one or more embodiments the pKa of hybrid polymer and/or a hydrophobic block of a hybrid polymer is 6.6 to 8.0, 6.6 to 7.5, 6.6 to 7.2, 6.6 to 7.0, 6.6 to 6.9, 6.6 to 6.8, or 6.6 to 6.7. In one or more embodiments the pKa of a hybrid polymer and/or a hydrophobic block of a hybrid polymer is 6.7 to 8.0, 6.7 to 7.5, 6.7 to 7.2, 6.7 to 7.0, 6.7 to 6.9, or 6.7 to 6.8. In one or more embodiments the pKa of a hybrid polymer and/or a hydrophobic block of a hybrid polymer is 6.8 to 8.0, 6.8 to 7.5, 6.8 to 7.2, 6.8 to 7.0, or 6.8 to 6.9. In one or more embodiments the pKa of a hybrid polymer and/or a hydrophobic block of a hybrid polymer is 6.9 to 8.0, 6.9 to 7.5, 6.9 to 7.2, or 6.9 to 7.0. In one or more embodiments the pKa of a hybrid polymer and/or a hydrophobic block of a hybrid polymer is 7.0 to 8.0, 7.0 to 7.5, or 7.0 to 7.2. In one or more embodiments the pKa of a hybrid polymer and/or a hydrophobic block of a hybrid polymer is 7.2 to 8.0, 7.2 to 7.5, or 7.5 to 8.0.

The hybrid polymer and/or a hydrophobic block of the present disclosure include protonatable nitrogen atoms. For example, the quinine moiety of a quinine-containing monomer has two nitrogen atoms that can be protonated one at the quinuclidine group (pKₐ=8.5) and the other at the quinoline ring (pKₐ=4.1). The protonation of a first nitrogen in a hybrid polymer of block can affect the protonation of the second nitrogen in a hybrid polymer or block. The Hill coefficient (n_{Hill}) is a measure of cooperativity in the protonation-deprotonation process. A value of n_{Hill}˜1 implies that the amine groups in a polymer undergo protonation (or deprotonation) independent of other amine groups in their proximity. Whereas n_{Hill}>1 suggests positive cooperativity in protonation (or deprotonation), which means protonation (or deprotonation) of one amine group facilitates protonation (or deprotonation) of the surrounding amine groups.

The hybrid polymer and/or a hydrophobic block of the present disclosure may have a variety of n_{Hill} values. In one or more embodiments, a hybrid polymer and/or a hydrophobic block has an n_{Hill} value of 0.5 or greater, 0.7 or greater, 1.0 or greater, 1.1 or greater, 1.2 or greater, 1.3 or greater, 1.4 or greater, 1.5 or greater, 1.6 or greater, 1.7 or greater, 1.8 or greater, 1.9 or greater, 2.0 or greater, 2.25 or greater, 2.50 or greater, 2.75 or greater, 3.0 or greater, 3.25 or greater, 3.5 or greater, 3.75 or greater, 4.0 or greater, 4.25 or greater, 4.5 or greater, or 4.75 or greater. In one or more embodiments, a hybrid polymer and/or a hydrophobic block has an n_{Hill} value of 5.0 or less, 4.75 or less, 4.5 or less, 4.25 or less, 4.0 or less, 3.75 or less, 3.5 or less, 3.25 or less, 3.0 or less, 2.75 or less, 2.5 or less, 2.25 or less, 2.0 or less, 1.9 or less, 1.8 or less, 1.7 or less, 1.6 or less, 1.5 or less, 1.4 or less, 1.3 or less, 1.2 or less, 1.1 or less, 1.0 or less, or 0.7 or less. In one or more embodiments, a hybrid polymer and/or a hydrophobic block has an n_{Hill} value of 0.5 to 5.0, 0.5 to 4.5, 0.5 to 4.0, 0.5 to 3.5, 0.5 to 3.0, 0.5 to 2.5, 0.5 to 2.2, 0.5 to 2.0, 0.5 to 1.9, 0.5 to 1.8, 0.5 to 1.7, 0.5 to 1.6, 0.5 to 1.5, 0.5 to 1.4, 0.5 to 1.3, 0.5 to 1.2, 0.5 to 1.1, 0.5 to 1.0, or 0.5 to 0.7. In one or more embodiments, a hybrid polymer and/or a hydrophobic block has an n_{Hill} value of 0.7 to 5.0, 0.7 to 4.5, 0.7 to 4.0, 0.7 to 3.5, 0.7 to 3.0, 0.7 to 2.5, 0.7 to 2.2, 0.7 to 2.0, 0.7 to 1.9, 0.7 to 1.8, 0.7 to 1.7, 0.7 to 1.6, 0.7 to 1.5, 0.7 to 1.4, 0.7 to 1.3, 0.7 to 1.2, 0.7 to 1.1, or 0.7 to 1.0. In one or more embodiments, a hybrid polymer and/or a hydrophobic block has an n_{Hill} value of 1.0 to 5.0, 1.0 to 4.5, 1.0 to 4.0, 1.0 to 3.5, 1.0 to 3.0, 1.0 to 2.5, 1.0 to 2.2, 1.0 to 2.0, 1.0 to 1.9, 1.0 to 1.8, 1.0 to 1.7, 1.0 to 1.6, 1.0 to 1.5, 1.0 to 1.4, 1.0 to 1.3, 1.0 to 1.2, or 1.0 to 1.1. In one or more embodiments, a hybrid polymer and/or a hydrophobic block has an n_{Hill} value of 1.1 to 5.0, 1.1 to 4.5, 1.1 to 4.0, 1.1 to 3.5, 1.1 to 3.0, 1.1 to 2.5, 1.1 to 2.2, 1.1 to 2.0, 1.1 to 1.9, 1.1 to 1.8, 1.1 to 1.7, 1.1 to 1.6, 1.1 to 1.5, 1.1 to 1.4, 1.1 to 1.3, or 1.1 to 1.2. In one or more embodiments, a hybrid polymer and/or a hydrophobic block has an n_{Hillvalue} of 1.2 to 5.0, 1.2 to 4.5, 1.2 to 4.0, 1.2 to 3.5, 1.2 to 3.0, 1.2 to 2.5, 1.2 to 2.2, 1.2 to 2.0, 1.2 to 1.9, 1.2 to 1.8, 1.2 to 1.7, 1.2 to 1.6, 1.2 to 1.5, 1.2 to 1.4, or 1.2 to 1.3. In one or more embodiments, a hybrid polymer and/or a hydrophobic block has an n_{Hillvalue} of 1.3 to 5.0, 1.3 to 4.5, 1.3 to 4.0, 1.3 to 3.5, 1.3 to 3.0, 1.3 to 2.5, 1.3 to 2.2, 1.3 to 2.0, 1.3 to 1.9, 1.3 to 1.8, 1.3 to 1.7, 1.3 to 1.6, 1.3 to 1.5, or 1.3 to 1.4. In one or more embodiments, a hybrid polymer and/or a hydrophobic block has an n_{Hill} value of 1.4 to 5.0, 1.4 to 4.5, 1.4 to 4.0, 1.4 to 3.5, 1.4 to 3.0, 1.4 to 2.5, 1.4 to 2.2, 1.4 to 2.0, 1.4 to 1.9, 1.4 to 1.8, 1.4 to 1.7, 1.4 to 1.6, or 1.4 to 1.5. In one or more embodiments, a hybrid polymer and/or a hydrophobic block has an n_{Hillvalue} of 1.5 to 5.0, 1.5 to 4.5, 1.5 to 4.0, 1.5 to 3.5, 1.5 to 3.0, 1.5 to 2.5, 1.5 to 2.2, 1.5 to 2.0, 1.5 to 1.9, 1.5 to 1.8, 1.5 to 1.7, or 1.5 to 1.6. In one or more embodiments, a hybrid polymer and/or a hydrophobic block has an n_{Hill} value of 1.6 to 5.0, 1.6 to 4.5, 1.6 to 4.0, 1.6 to 3.5, 1.6 to 3.0, 1.6 to 2.5, 1.6 to 2.2, 1.6 to 2.0, 1.6 to 1.9, 1.6 to 1.8, or 1.6 to 1.7. In one or more embodiments, a hybrid polymer and/or a hydrophobic block has an n_{Hill} value of 1.7 to 5.0, 1.7 to 4.5, 1.7 to 4.0, 1.7 to 3.5, 1.7 to 3.0, 1.7 to 2.5, 1.7 to 2.2, 1.7 to 2.0, 1.7 to 1.9, or 1.7 to 1.8. In one or more embodiments, a hybrid polymer and/or a hydrophobic block has an n_{Hillvalue} of 1.8 to 5.0, 1.8 to 4.5, 1.8 to 4.0, 1.8 to 3.5, 1.8 to 3.0, 1.8 to 2.5, 1.8 to 2.2, 1.8 to 2.0, or 1.8 to 1.9. In one or more embodiments, a hybrid polymer and/or a hydrophobic block has an n_{Hill} value of 1.9 to 5.0, 1.9 to 4.5, 1.9 to 4.0, 1.9 to 3.5, 1.9 to 3.0, 1.9 to 2.5, 1.9 to 2.2, or 1.9 to 2.0. In one or more embodiments, a hybrid polymer and/or a hydrophobic block has an n_{Hill} value of 2.0 to 5.0, 2.0 to 4.5, 2.0 to 4.0, 2.0 to 3.5, 2.0 to 3.0, 2.0 to 2.5, 2.0 to 2.2, or 2.2 to 2.5.

A hybrid polymer of the present disclosure includes a hydrophilic block polymerized from at least a hydrophilic monomer. In one or more embodiments, the hydrophilic block may include poly(ethylene glycol) (PEG), poly(ethylene oxide) (PEO), poly(vinylpyrrolidone) (PVP), poly(acrylic acid) (PAA), maleic acid, poly(vinyl alcohol) (PVA), hydrophilic polyurethanes, poly(2-hydroxyethyl) methacrylate (pHEMA), poly(ethylene glycol) methacrylate, poly(2-methoxyethyl methacrylate), poly(2-ethoxyethyl methacrylate), poly[2-(dimethylamino)ethyl]methacrylate (DMAEMA), poly[3-(dimethylamino)propyl]methacrylamide (DMAPMA), poly(propylene glycol), poly(sarcosine), carbohydrates, gelatins, salts thereof, or mixtures thereof.

“PEG,” “polyethylene glycol,” and “poly(ethylene glycol)” refers to a polymer or block having the repeat unit —(CH₂CH₂O)—. The term “PEG” includes polymers of bocks having various terminal or end capping groups and so forth. With respect to specific forms, the PEG can take any number of a variety of molecular weights, as well as structures or geometries such as branched, linear, forked, multifunctional, dendrimeric, and the like.

In one or more embodiments, the hydrophilic block in a hybrid polymers of the present disclosure includes PEG. The hydrophilic block in a hybrid polymer may include polyethylene glycols such as PEG 300, PEG 400, PEG 600 PEG 800, PEG 1000, PEG 1500, PEG 2000, PEG 3000, PEG 3350, PEG 4000, PEG 5500, PEG 6000, PEG 8000, PEG 10000, PEG 12000, PEG 20000, PEG 35000, PEG 40000, PEG 50000, PEG 60000, PEG 70000, PEG 80000, PEG 90000, and PEG 100000 where the number after PEG indicates the average molecular weight. In one or more embodiments, the hydrophilic block in a hybrid polymer includes PEG having an average molecular weight of 1000 to 100000, such as, for example, 1000 to 10000, 5000 to 15000, or 8000 to 12000. The hydrophilic block in a hybrid polymer may include polyethylene glycols such as statistical ethylene oxide/propylene oxide-copolymerisates, such as the Polyglykol P41, statistical ethylene oxide/propylene oxide-copolymerisate-monobutyl ethers, such as the Polyglykol B11, polyalkylene glycols such as the Polyglykol PR-types (for example, Polyglykol—PR 300, Polyglykol—PR 450, Polyglykol—PR600 and Polyglykol—PR 1000), glycol esters, and mixtures and combinations thereof.

The hydrophilic block of a hybrid polymers of the present disclosure may be prepared from different monomers. For example, the monomer used for the preparation of the hydrophilic block may be selected from acrylic acid, maleic acid, hydroxyethyl methacrylate (HEMA), polyvinylpyrrolidone (PVP), polyvinyl alcohol (PVA), ethylene glycol, polyethylene glycols and derivatives thereof, such as poly(ethylene glycol) methacrylate, poly(2-methoxyethyl methacrylate), poly(2-ethoxyethyl methacrylate), DMAEMA, or DMAPMA, in order to impart desired properties into the polymer. A hydrophilic block may be a copolymer of neutralized methacrylic acids, such as sodium acrylate and hydroxyethyl methacrylate, vinylpyrrolidone, polyethylene glycols and derivatives thereof, such as poly(ethylene glycol) methacrylate, methoxyethyl methacrylate, ethoxy methacrylate, DMAEMA or DMAPMA.

In one or more embodiments, hydrophobic monomers may be present in the hydrophilic block, for example, in order to impart desired properties into the hydrophilic block. Likewise, it is possible to have a number of hydrophilic monomers, present in the hydrophobic block.

A hydrophilic block in the hybrid polymers of the present disclosure may include carbohydrate-containing repeat units. A hydrophilic block may be polymerized from a carbohydrate-containing monomer. A hydrophilic block may be polymerized from carbohydrate-containing monomers that are amenable to RAFT polymerization, for example, glucose, mannose, galactose, fucose derivatives, or combinations thereof. A carbohydrate-containing monomer can include a polymerizable group appended to the 1 position, the 2 position, or the 6 position. A carbohydrate-containing monomer can be the alpha or beta anomer. A carbohydrate-containing monomer may be a sugar amine substituted at the 2-position of the pyranose ring as shown below. In the chair conformation, for the numbering of the ring, the reference plane is chosen such that the lowest-numbered atom (usually C-1) is exoplanar. For example, for alpha-D-glucose, the numbering of the carbons on the pyranose ring is as follows:

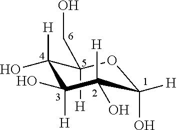

A carbohydrate-containing monomer may be a sugar amine substituted at the 2-position of the pyranose rings. The substituent at the 2-positon can include a polymerizable group. Examples of sugar amines substituted at the 2-positon of the pyranose ring include

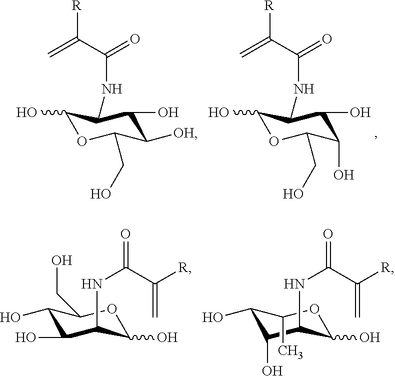

or an ionized version thereof, where R is H or methyl.

In one or more embodiments, a carbohydrate-containing monomer includes an N-acetyl substitution at the 2-position of the pyranose ring. The substituent at the 2-positon can include a polymerizable group. Examples of carbohydrate-containing monomers having an N-acetyl substitution at the 2-position of the pyranose ring include

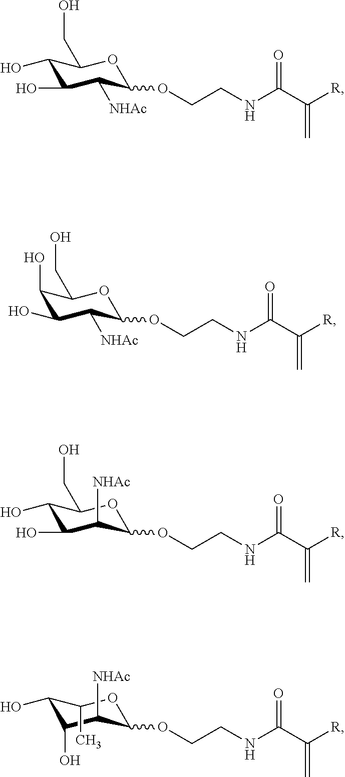

or an ionized version thereof, where R is H or methyl.

In one or more embodiments, a carbohydrate-containing monomer is substituted at the 1-position of the pyranose ring. The substituent at the 1-positon can include a polymerizable group. Examples of carbohydrate-containing monomers substituted at the 1-position of the pyranose ring include

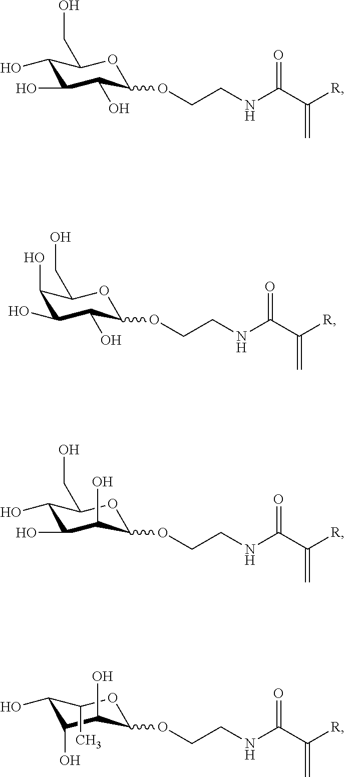

or an ionized version thereof, where R is H or methyl.

In one or more embodiments, a carbohydrate-containing monomer is a sugar dimer. Example sugar dimer monomers include

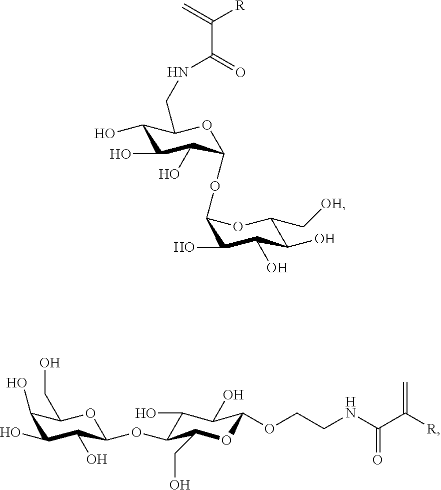

or an ionized version thereof, where R is a H or methyl.

In one or more embodiments, a carbohydrate-containing monomer is substituted at the 6-position. The substituent at the 6-positon can include a polymerization functional group. Examples of carbohydrate-containing monomer substituted at the 6-positon include

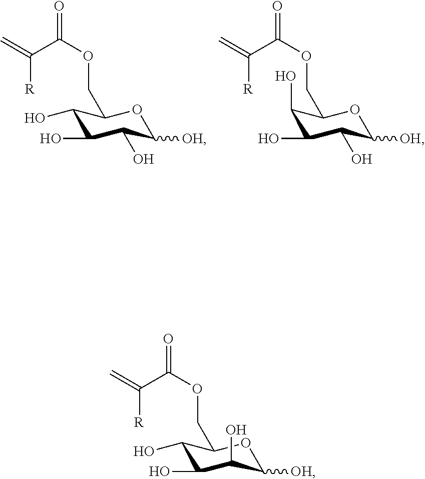

or an ionized version thereof, where R is H or methyl.

In one or more embodiments, a hydrophilic block is polymerized from at least a glucose-containing monomer. In one or more embodiments, a hydrophilic block is polymerized from at least

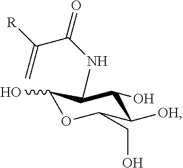

where R is H or CH₃. In one or more embodiments, a hydrophilic block includes

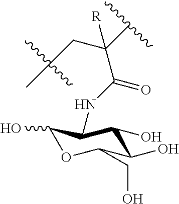

where R is H or CH₃.

In one or more embodiments, a hydrophilic block is polymerized from at least a galactose-containing monomer. In one or more embodiments, a hydrophilic block is polymerized from at least

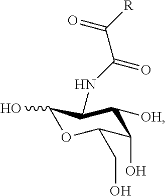

where R is H or CH₃. In one or more embodiments, a hydrophilic block includes

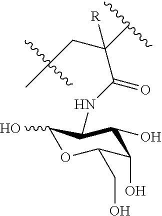

where R is H or CH₃.

Polymerization of a carbohydrate-containing monomer results in carbohydrate-containing repeat units. In one or more embodiments, a hydrophilic block of a hybrid polymers includes one or more of the following carbohydrate-containing repeat units:

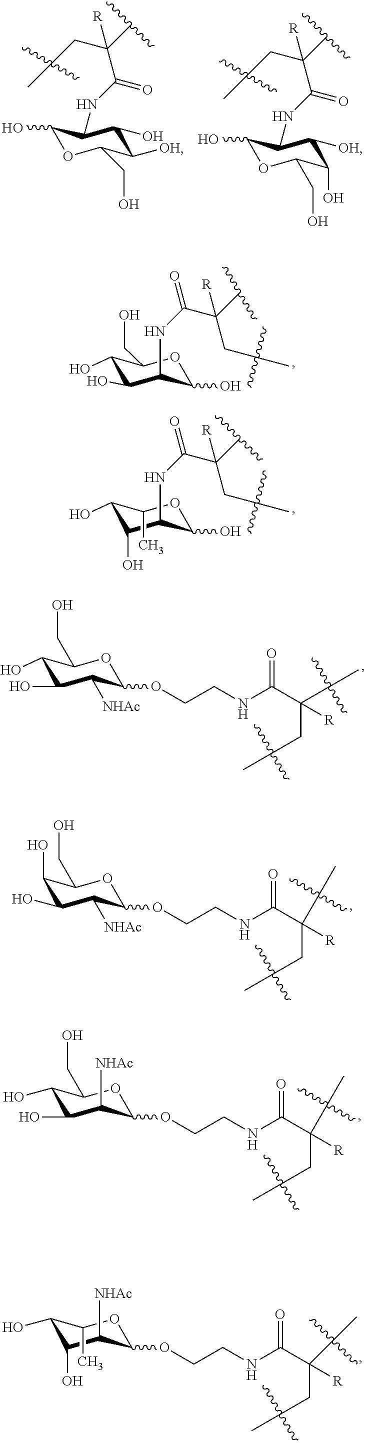

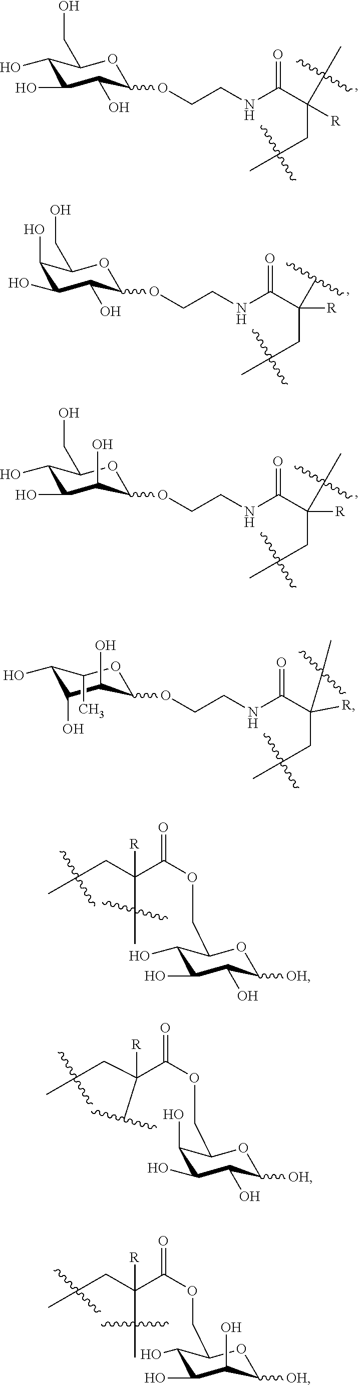

or an ionized form thereof, where R is H or methyl.

In one or more embodiments, the Mw and/or the Mn of a hydrophilic block of a hybrid polymer is 1 kilodalton (kDa) or greater, 2 kDa or greater, 3 kDa or greater, 4 kDa or greater, 5 kDa or greater, 10 kDa or greater, 15 kDa or greater, 20 kDa or greater, 25 kDa or greater, 30 kDa or greater, 35 kDa or greater, 40 kDa or greater, 45 kDa or greater, 50 kDa or greater, 75 kDa or greater, 100 kDa or greater, 125 kDa or greater, 150 kDa or greater, 175 kDa or greater, 200 kDa or greater, 250 kDa or greater, 300 kDa or greater, 350 kDa or greater, 400 kDa or greater, or 450 kDa or greater. In one or more embodiments, the Mw and/or the Mn of a hydrophilic block of a hybrid polymer 500 kDa or less, 450 kDa or less, 400 kDa or less, 350 kDa or less, 300 kDa or less, 250 kDa or less, 200 kDa or less, 175 kDa or less, 150 kDa or less, 125 kDa or less, 100 kDa or less, 75 kDa or less, 50 kDa or less, 40 kDa or less, 30 kDa or less, 25 kDa or less, 20 kDa or less, or 10 kDa or less. In one or more embodiments, the Mw and/or the Mn of a hydrophilic block of a hybrid polymer 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the molar mass of the carbohydrates in the hydrophilic block is 10 kDa to 500 kDa, 10 kDa to 450 kDa, 10 kDa to 400 kDa, 10 kDa to 350 kDa, 10 kDa to 300 kDa, 10 kDa to 250 kDa, 10 kDa to 200 kDa, 10 kDa to 150 kDa, 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mw and/or the Mn of a hydrophilic block of a hybrid polymer is 20 kDa to 500 kDa, 20 kDa to 450 kDa, 20 kDa to 400 kDa, 20 kDa to 350 kDa, 20 kDa to 300 kDa, 20 kDa to 250 kDa, 20 kDa to 200 kDa, 20 kDa to 150 kDa, 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mw and/or the Mn of a hydrophilic block of a hybrid polymer is 25 kDa to 500 kDa, 25 kDa to 450 kDa, 25 kDa to 400 kDa, 25 kDa to 350 kDa, 25 kDa to 300 kDa, 25 kDa to 250 kDa, 25 kDa to 200 kDa, 25 kDa to 150 kDa, 25 kDa to 100 kDa, 25 kDa to 75 kDa, 25 kDa to 50 kDa, 25 kDa to 40 kDa, or 25 kDa to 30 kDa. In one or more embodiments, the Mw and/or the Mn of a hydrophilic block of a hybrid polymer is 30 kDa to 500 kDa, 30 kDa to 450 kDa, 30 kDa to 400 kDa, 30 kDa to 350 kDa, 30 kDa to 300 kDa, 30 kDa to 250 kDa, 30 kDa to 200 kDa, 30 kDa to 150 kDa, 30 kDa to 100 kDa, 30 kDa to 75 kDa, 30 kDa to 50 kDa, or 30 kDa to 40 kDa. In one or more embodiments, the Mw and/or the Mn of a hydrophilic block of a hybrid polymer is 40 kDa to 500 kDa, 40 kDa to 450 kDa, 40 kDa to 400 kDa, 40 kDa to 350 kDa, 40 kDa to 300 kDa, 40 kDa to 250 kDa, 40 kDa to 200 kDa, 40 kDa to 150 kDa, 40 kDa to 100 kDa, 40 kDa to 75 kDa, or 40 kDa to 50 kDa. In one or more embodiments, the Mw and/or the Mn of a hydrophilic block of a hybrid polymer is 50 kDa to 500 kDa, 50 kDa to 450 kDa, 50 kDa to 400 kDa, 50 kDa to 350 kDa, 50 kDa to 300 kDa, 50 kDa to 250 kDa, 50 kDa to 200 kDa, 50 kDa to 150 kDa, 50 kDa to 100 kDa or 50 kDa to 75 kDa. In one or more embodiments, the Mw and/or the Mn of a hydrophilic block of a hybrid polymer is 75 kDa to 500 kDa, 75 kDa to 450 kDa, 75 kDa to 400 kDa, 75 kDa to 350 kDa, 75 kDa to 300 kDa, 75 kDa to 250 kDa, 75 kDa to 200 kDa, 75 kDa to 150 kDa, or 75 kDa to 100 kDa. In one or more embodiments, the Mw and/or the Mn of a hydrophilic block of a hybrid polymer is 100 kDa to 500 kDa, 100 kDa to 450 kDa, 100 kDa to 400 kDa, 100 kDa to 350 kDa, 100 kDa to 300 kDa, 100 kDa to 250 kDa, 100 kDa to 200 kDa, or 100 kDa to 150 kDa. In one or more embodiments, the Mw and/or the Mn of a hydrophilic block of a hybrid polymer is 150 kDa to 500 kDa, 150 kDa to 450 kDa, 150 kDa to 400 kDa, 150 kDa to 350 kDa, 150 kDa to 300 kDa, 150 kDa to 250 kDa, or 150 kDa to 200 kDa. In one or more embodiments, the Mw and/or the Mn of a hydrophilic block of a hybrid polymer is 200 kDa to 500 kDa, 200 kDa to 450 kDa, 200 kDa to 400 kDa, 200 kDa to 350 kDa, 200 kDa to 300 kDa, or 200 kDa to 250 kDa. In one or more embodiments, the Mw and/or the Mn of a hydrophilic block of a hybrid polymer is 250 kDa to 500 kDa, 250 kDa to 450 kDa, 250 kDa to 400 kDa, 250 kDa to 350 kDa, or 250 kDa to 300 kDa. In one or more embodiments, the Mw and/or the Mn of a hydrophilic block of a hybrid polymer is 300 kDa to 500 kDa, 300 kDa to 450 kDa, 300 kDa to 400 kDa, or 300 kDa to 350 kDa. In one or more embodiments, the Mw and/or the Mn of a hydrophilic block of a hybrid polymer is 350 kDa to 500 kDa, 350 kDa to 450 kDa, or 350 kDa to 400 kDa. In one or more embodiments, the Mw and/or the Mn of a hydrophilic block of a hybrid polymer is 400 kDa to 500 kDa, 400 kDa to 450 kDa, 400 kDa to 500 kDa, or 450 kDa to 500 kDa.

In embodiments, the hybrid polymer of the present disclosure is of formula XXXVII:

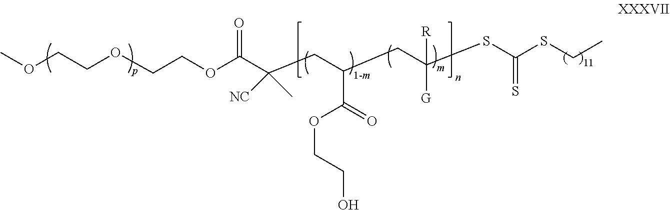

where m is the mole-% (mol-%) of quinoline-containing repeat units formed from a quinoline-containing monomer, R is H or CH₃, p is 5 to 2500, and n is 10 to 1000.

For polymers having square brackets with parentheses within the square brackets, it is understood that the square brackets indicate a block made of monomers. Within the square brackets, the parentheses, with fractional numbers, indicate the monomer components that make up that block. The components that make up the block within the square brackets can be arranged in any order. For example, in one or more embodiments, the components that make up the block within the square brackets can be arranged in a statistical nature. For example, in formula XXXVII, the hydrophobic block includes two types of monomers (HEA) and G at m and 1-m fraction.

In one or more embodiments, G may be a quinoline-containing repeat unit formed from a quinoline-containing monomer of the general formula X, X(a), or X(b). In one or more embodiments, G is

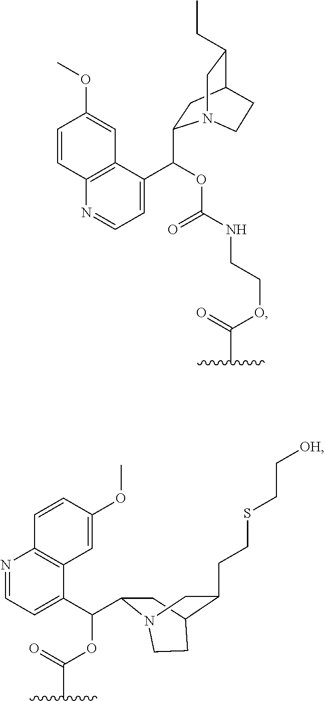

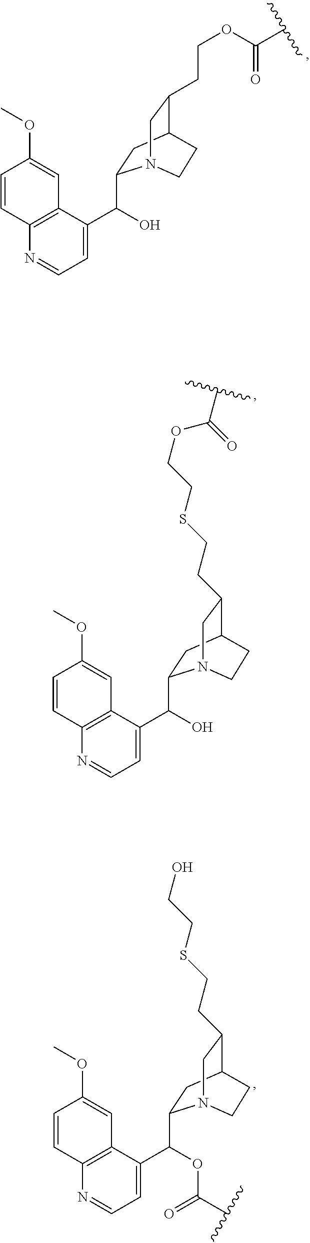

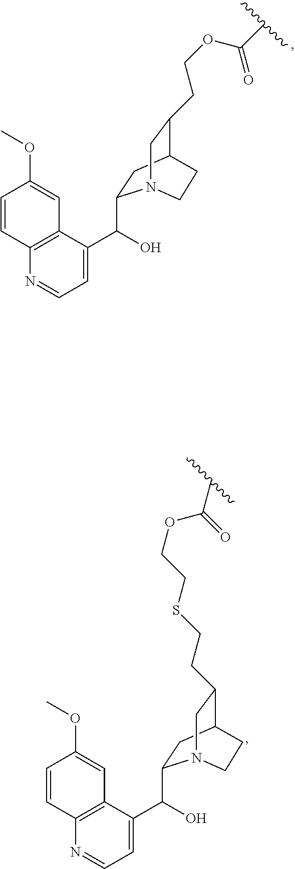

or an ionized version thereof.

p is 5 to 2500. p can be 5 or more, 10 or more, 25 or more, 50 or more, 75 or more, 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, 500 or more, 525 or more, 550 or more, 575 or more, 600 or more, 625 or more, 650 or more, 675 or more, 700 or more, 725 or more, 750 or more, 775 or more 800 or more, 825 or more, 850 or more, 875 or more, 900 or more, 925 or more, 950 or more, 975 or more, 1000 or more, 1100 or more, 1200 or more, 1300 or more, 1400 or more 1500 or more, 1600 or more, 1700 or more, 1800 or more, 1900 or more, 2000 or more, 2100 or more, 2200 or more, 2300 or more, or 2400 or more. p can be 2500 or less, 2400 or less, 2300 or less, 2200 or less, 2100 or less, 2000 or less, 1900 or less, 1800 or less, 1700 or less, 1600 or less, 1500 or less, 1400 or less, 1300 or less, 1200 or less, 1100 or less, 1000 or less, 975 or less, 950 or less, 925 or less, 900 or less, 875 or less, 850 or less, 825 or less, 800 or less, 775 or less, 750 or less, 725 or less, 700 or less, 675 or less, 650 or less, 625 or less, 600 or less, 575 or less, 550 or less, 525 or less, 500 or less, 475 or less, 450 or less, 425 or less, 400 or less, 375 or less, 350 or less, 325 or less, 300 or less, 275 or less, 250 or less, 225 or less, 200 or less, 175 or less, 150 or less, 120 or less, 100 or less, 75 or less, 50 or less, 25 or less, or 10 or less.

n is 10 to 1000. n can be 5 or more, 10 or more, 25 or more, 50 or more, 75 or more, 100 or more, 125 or more, 150 or more, 175 or more, 200 or more, 225 or more, 250 or more, 275 or more, 300 or more, 325 or more, 350 or more, 375 or more, 400 or more, 425 or more, 450 or more, 475 or more, 500 or more, 525 or more, 550 or more, 575 or more, 600 or more, 625 or more, 650 or more, 675 or more, 700 or more, 725 or more, 750 or more, 775 or more 800 or more, 825 or more, 850 or more, 875 or more, 900 or more, 925 or more, 950 or more, or 975 or more. n can be 1000 or less, 975 or less, 950 or less, 925 or less, 900 or less, 875 or less, 850 or less, 825 or less, 800 or less, 775 or less, 750 or less, 725 or less, 700 or less, 675 or less, 650 or less, 625 or less, 600 or less, 575 or less, 550 or less, 525 or less, 500 or less, 475 or less, 450 or less, 425 or less, 400 or less, 375 or les, 350 or less, 325 or less, 300 or less, 275 or less, 250 or less, 225 or less, 200 or less, 175 or less, 150 or less, 120 or less, 100 or less, 75 or less, 50 or less, 25 or less, or 10 or less.

m is the mol-% of quinoline-containing repeat units. m can be 0.05 or greater, 0.10 or greater, 0.15 or greater, 0.20 or greater, 0.25 or greater, 0.30 or greater, 0.35 or greater, 0.40 or greater, 0.45 or greater, 0.50 or greater, 0.55 or greater, 0.60 or greater, 0.65 or greater, 0.70 or greater, 0.75 or greater, 0.80 or greater, 0.85 or greater, 0.90 or greater, or 0.95 or greater. m can be 1 or less, 0.95 or less, 0.90 or less, 0.85 or less, 0.80 or less, 0.75 or less, 0.70 or less, 0.65 or less, 0.60 or less, 0.55 or less, 0.50 or less, 0.45 or less, 0.40 or less, 0.35 or less, 0.30 or less, 0.25 or less, 0.20 or less, 0.15 or less, or 0.10 or less.

In embodiments, the hybrid polymer of the present disclosure is of formula XXXVIII:

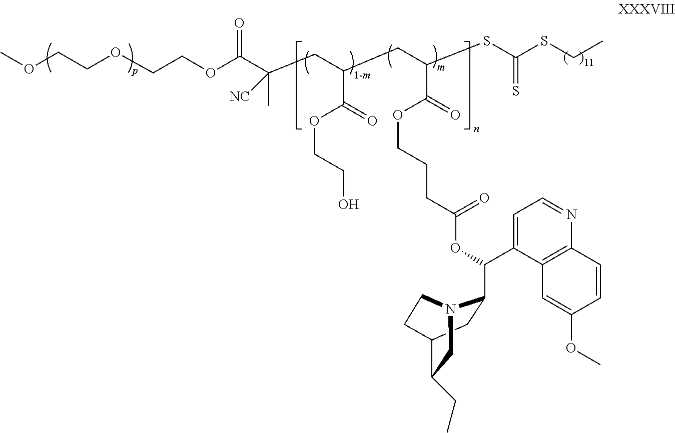

where p, m, and n are defined herein.

In embodiments, the hybrid polymer of the present disclosure is of formula C:

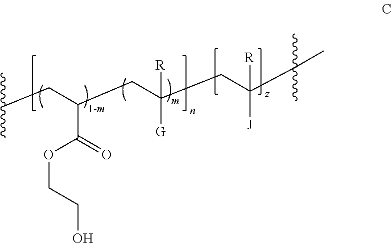

where m, n, and G are defined herein; each R is independently H or CH₃; n is 10 to 1000, z is 10 to 2500, and J is a carbohydrate-containing repat unit formed from a carbohydrate-containing monomer. G may be any G defined herein. z can be any value of n defined herein. In one or more embodiments, J can be

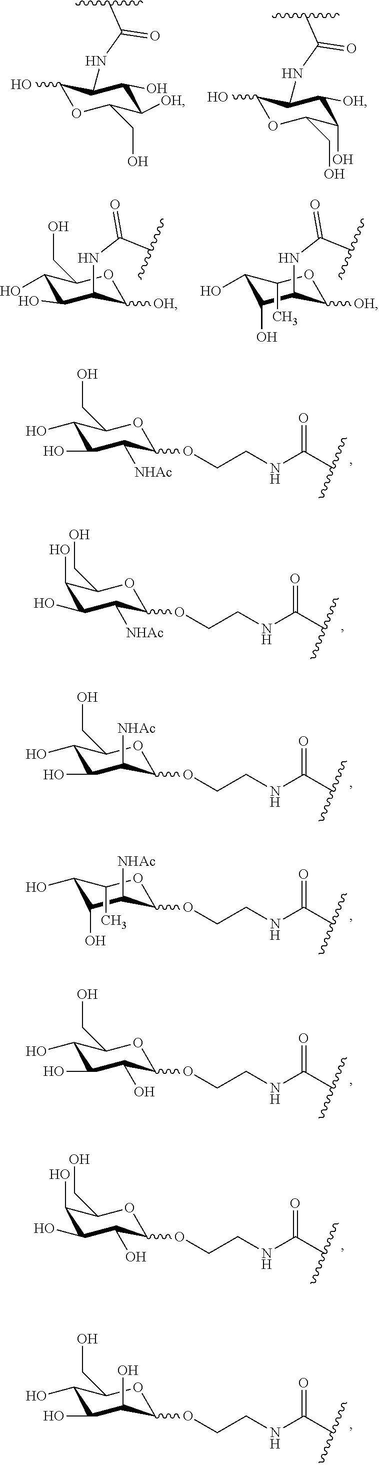

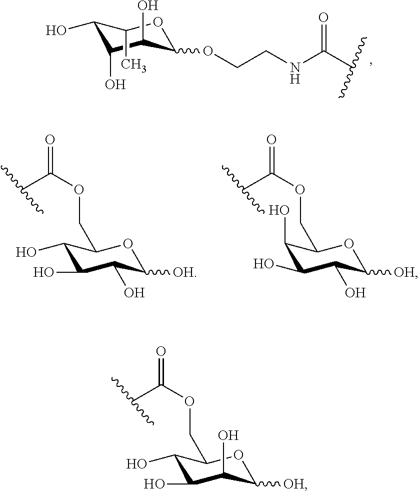

or an ionized form thereof.

In embodiments, the hybrid polymer of the present disclosure is of formula CI:

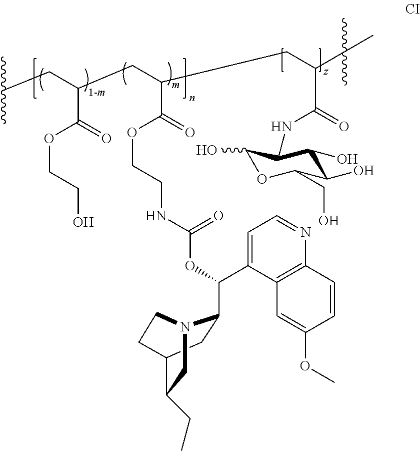

where m, n, and z are defined herein.

In embodiments, the hybrid polymer of the present disclosure is of formula CI:

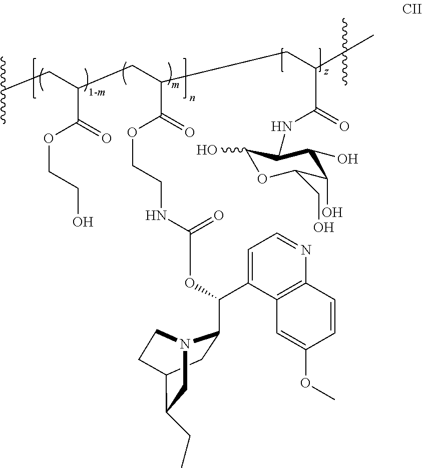

where m, n, and z are defined herein.

### Hydrophobic Polymers

The present disclosure also describes hydrophobic polymers. A hydrophobic polymer of the present disclosure includes quinoline-containing repeat units. A hydrophobic polymer of the present disclosure can be a copolymer polymerized from a quinoline-containing monomer and a second monomer. A hydrophobic polymer of the present disclosure can be any hydrophobic block of a hybrid polymer described herein.

A hydrophobic polymer of the present disclosure includes quinoline-containing repeat units. A quinoline-containing monomer used in the polymerization of a hydrophobic polymer may be any quinoline-containing monomer described herein. For example, a hydrophobic polymer can be polymerized from a quinoline-containing monomer of formula X, X(a), or X(b). A hydrophobic polymer can include any quinoline-containing repeat unit described herein.

In one or more embodiments, a hydrophobic polymer is a copolymer polymerized from a quinoline-containing monomer and a second monomer. The second monomer can be any suitable second monomer, such as, for example, the second monomers described herein for inclusion in a hydrophobic block of a hybrid polymer. In one or more embodiments, the second monomer is 2-hydroxyethyl acrylate (HEA).

A hydrophobic polymer of the present disclosure can have any characteristic of a hydrophobic block of a hybrid polymer described herein. For example, a hydrophobic polymer of the present disclosure can have a number-average molecular weight, weight-average molecular weight, pKa, and/or Hill coefficient of a hydrophobic block of a hybrid polymer.

The amount of the quinoline-containing monomer in a hydrophobic polymer may affect the properties of the hydrophobic polymer. The mole percent (mol-%) of the quinoline-containing monomer in a hydrophobic polymer may be measured and calculated using 1H NMR via methods known in the art. In one or more embodiments, the amount of the quinoline-containing monomer in a hydrophobic polymer is 1 mol-% or greater, 5 mol-% or greater, 10 mol-% or greater, 15 mol-% or greater, 20 mol-% or greater, 25 mol-% or greater, 30 mol-% or greater, 60 mol-% or greater, 80 mol-% or greater. In one or more embodiments, the amount of the quinoline-containing monomer in a hydrophobic polymer is 100 mol-% or less, 80 mol-% or less, 60 mol-% or less, 30 mol-% or less, 25 mol-% or less, 20 mol-% or less, or 15 mol-% or less, 10 mol-% or less, or 5 mol-% or less. In one or more embodiments, the amount of the quinoline-containing monomer in a hydrophobic polymer is 1 mol-% to 100 mol-%, 1 mol-% to 80 mol-%, 1 mol-% to 60 mol-%, 1 mol-% to 30 mol-%, 1 mol-% to 25 mol-%, 1 mol-% to 20 mol-%, 1 mol-% to 15 mol-%, 1 mol-% to 10 mol-%, or 1 mol-% to 5 mol-%. In one or more embodiments, the amount of the quinoline-containing monomer in a hydrophobic polymer is 5 mol-% to 100 mol-%, 5 mol-% to 80 mol-%, 5 mol-% to 60 mol-%, 5 mol-% to 30 mol-%, 5 mol-% to 25 mol-%, 5 mol-% to 20 mol-%, 5 mol-% to 15 mol-%, or 5 mol-% to 10 mol-%. In one or more embodiments, the amount of the quinoline-containing monomer in a hydrophobic polymer is 10 mol-% to 100 mol %, 10 mol-% to 80 mol %, 10 mol-% to 60 mol %, 10 mol-% to 30 mol-%, 10 mol-% to 25 mol-%, 10 mol-% to 20 mol-%, or 10 mol-% to 15 mol-%. In one or more embodiments, the amount of the quinoline-containing monomer in a hydrophobic polymer is 15 mol-% to 100 mol-%, 15 mol-% to 80 mol-%, 15 mol-% to 60 mol-%, 15 mol-% to 30 mol-%, 15 mol-% to 25 mol-%, or 15 mol-% to 20 mol-%. In one or more embodiments, the amount of the quinoline-containing monomer in a hydrophobic polymer is 20 mol-% to 100 mol-%, 20 mol-% to 80 mol %, 20 mol-% to 60 mol %, 20 mol-% to 30 mol %, or 20 mol-% to 25 mol-%. In one or more embodiments, the amount of the quinoline-containing monomer in a hydrophobic polymer is 25 mol-% to 100 mol-%, 25 mol-% to 80 mol-%, 25 mol-% to 60 mol-%, or 25 mol-% to 30 mol-%. In one or more embodiments, the amount of the quinoline-containing monomer in a hydrophobic polymer is 30 mol-% to 100 mol-%, 30 mol-% to 80 mol-%, or 30 mol-% to 60 mol-%. In one or more embodiments, the amount of the quinoline-containing monomer in a hydrophobic polymer is 60 mol-% to 100 mol-% or 60 mol-% to 80 mol-%. In one or more embodiments, the amount of the quinoline-containing monomer in a hydrophobic polymer is 80 mol-% to 100 mol-%. In one or more embodiments, the amount of the quinoline-containing monomer in a hydrophobic polymer is 15 mol-% to 60 mol-%. In one or more embodiments, the amount of the quinoline-containing monomer in a hydrophobic polymer is 15 mol-% to 30 mol-%.

In embodiments, a hydrophobic polymer of the present disclosure is of formula XXXIX:

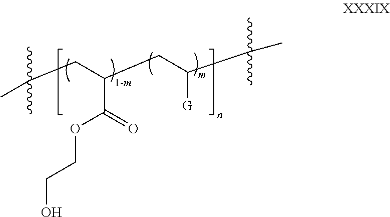

or an ionized form thereof, where m, G, and n are defined herein.

In embodiments, a hydrophobic polymer of the present disclosure is of formula XL:

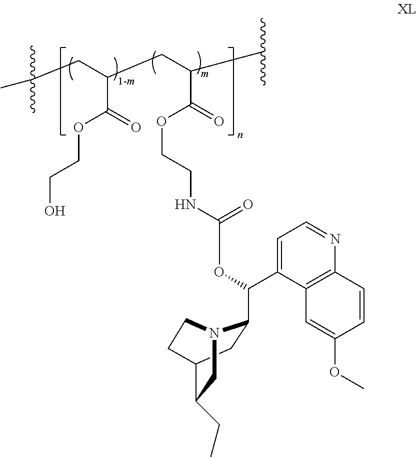

or an ionized form thereof, where m and n are defined herein.

In one or more embodiments, the present disclosure describes transfection agents. A transfection agent is a material that can make a complex with an oligonucleotide to facilitate the transfer of the oligonucleotide from the extracellular space across the cell membrane into the intracellular space. The hybrid polymers of the present disclosure, the hydrophobic polymers of the present disclosure, and mixtures of the hybrid polymers and the hydrophobic polymers can be transfection agents.

A transfection agent may be used to facilitate transfection. Transfection is the process of introducing oligonucleotides into a cell using nonviral methods. A cell that has successfully undergone transfection, that is, the oligonucleotide is located within the cell (cytoplasm or nuclease), is a transfected cell.

A complex of an oligonucleotide and a transfection agent is a transfection complex, also referred to as a polyplex, a polyplex complex, or a polyplex transfection complex. In the present disclosure, the transfection agent includes a hybrid polymer, a hydrophobic polymer, or both. An oligonucleotide can associate with (e.g., binds to) a polymer via electrostatic interactions between the negatively charged oligonucleotide backbone and the protonated amines (e.g., the quinuclidine nitrogen of the polymer). In one or more embodiments, an oligonucleotide includes plasmid DNA. Additionally, intercalation of the quinoline-containing monomers and an oligonucleotide may contribute to binding. A polyplex can facilitate the transport of the oligonucleotide across the cell membrane. However, quinoline-containing polymers are hydrophobic, and hence their polyplexes are not colloidally stable. In other words, the complexes formed due to the binding of quinoline-containing polymers and nucleic acids, such as mRNA and pDNA, aggregate over time to larger sizes (diameter>1000 nanometers). In one or more embodiments, the present disclosure describes that mixing the hybrid polymers of the present disclosure with a hydrophobic polymer, such as the hydrophobic polymers described herein, can inhibit or decrease the aggregation of polyplex particles. In embodiments, the mixing of hybrid polymers with hydrophobic polymers allows an oligonucleotide to form a polyplex while the hydrophilic block of the hybrid polymer protects the coating on the final polyplex particles keeping the particle size under 200 nanometers.

In one or more embodiments, a polyplex transfection complex includes a hybrid polymer of the present disclosure and a hydrophobic polymer of the present disclosure. In one or more embodiments, the amount of hybrid polymer in the polyplex transfection complex may be 80% or less, 70% or less, 60% or less, 50% or less, 45% or less, 40% or less, 35% or less, 30% or less, 25% or less, 20% or less, 15% or less, 10% or less, or 5% or less, relative to the total amount of polymer (the amount of the hybrid polymer plus the amount of the hydrophobic polymer) in the polyplex transfection complex. In one or more embodiments, the amount of hybrid polymer in the polyplex transfection complex may be 80%, 70%, 60%, 50%, 45%, 40%, 35%, 30%, 25%, 20%, 15%, 10%, or 5%, relative to the total amount of polymer in the polyplex transfection complex.

The weight or mole ratio of a hydrophobic polymer to a hybrid polymer in a polyplex transfection complex can vary. The weight or mole ratio of the a hydrophobic polymer to a hybrid polymer in a polyplex transfection complex can be 0.01 parts or greater hydrophobic polymer for every 1 part hybrid polymer, 0.02 parts or greater hydrophobic polymer for every 1 part hybrid polymer, 0.03 parts or greater hydrophobic polymer for every 1 part hybrid polymer, 0.04 parts or greater hydrophobic polymer for every 1 part hybrid polymer, 0.05 parts or greater hydrophobic polymer for every 1 part hybrid polymer, 0.06 parts or greater hydrophobic polymer for every 1 part hybrid polymer, 0.07 parts or greater hydrophobic polymer for every 1 part hybrid polymer, 0.08 parts or greater hydrophobic polymer for every 1 part hybrid polymer, 0.09 parts or greater hydrophobic polymer for every 1 part hybrid polymer, 0.1 parts or greater hydrophobic polymer for every 1 part hybrid polymer, 0.2 parts or greater hydrophobic polymer for every 1 part hybrid polymer, 0.3 parts or greater hydrophobic polymer for every 1 part hybrid polymer, 0.4 parts or greater hydrophobic polymer for every 1 part hybrid polymer, 0.5 parts or greater hydrophobic polymer for every 1 part hybrid polymer, 0.6 parts or greater hydrophobic polymer for every 1 part hybrid polymer, 0.7 parts or greater hydrophobic polymer for every 1 part hybrid polymer, 0.8 parts or greater hydrophobic polymer for every 1 part hybrid polymer, 0.9 parts or greater hydrophobic polymer for every 1 part hybrid polymer, 2 parts or greater hydrophobic polymer for every 1 part hybrid polymer, 3 parts or greater hydrophobic polymer for every 1 part hybrid polymer, 4 parts or greater hydrophobic polymer for every 1 part hybrid polymer, 5 parts or greater hydrophobic polymer for every 1 part hybrid polymer, 6 parts or greater hydrophobic polymer for every 1 part hybrid polymer, 7 parts or greater hydrophobic polymer for every 1 part hybrid polymer, 8 parts or greater hydrophobic polymer for every 1 part hybrid polymer, 9 parts or greater hydrophobic polymer for every 1 part hybrid polymer, 10 parts or greater hydrophobic polymer for every 1 part hybrid polymer, 11 parts or greater hydrophobic polymer for every 1 part hybrid polymer, 12 parts or greater hydrophobic polymer for every 1 part hybrid polymer, 13 parts or greater hydrophobic polymer for every 1 part hybrid polymer, 14 parts or greater hydrophobic polymer for every 1 part hybrid polymer, 15 parts or greater hydrophobic polymer for every 1 part hybrid polymer, 16 parts or greater hydrophobic polymer for every 1 part hybrid polymer, 17 parts or greater hydrophobic polymer for every 1 part hybrid polymer, 18 parts or greater hydrophobic polymer for every 1 part hybrid polymer, 19 parts or greater hydrophobic polymer for every 1 part hybrid polymer, 20 parts or greater hydrophobic polymer for every 1 part hybrid polymer, 30 parts or greater hydrophobic polymer for every 1 part hybrid polymer, 40 parts or greater hydrophobic polymer for every 1 part hybrid polymer, 50 parts or greater hydrophobic polymer for every 1 part hybrid polymer, 60 parts or greater hydrophobic polymer for every 1 part hybrid polymer, 70 parts or greater hydrophobic polymer for every 1 part hybrid polymer, 80 parts or greater hydrophobic polymer for every 1 part hybrid polymer, or 90 parts or greater hydrophobic polymer for every 1 part hybrid polymer. The weight or mole ratio of the a hydrophobic polymer to a hybrid polymer in a polyplex transfection complex can be 100 parts or less hydrophobic polymer for every 1 part hybrid polymer, 90 parts or less hydrophobic polymer for every 1 part hybrid polymer, 80 parts or less hydrophobic polymer for every 1 part hybrid polymer, 70 parts or less hydrophobic polymer for every 1 part hybrid polymer, 60 parts or less hydrophobic polymer for every 1 part hybrid polymer, 50 parts or less hydrophobic polymer for every 1 part hybrid polymer, 40 parts or less hydrophobic polymer for every 1 part hybrid polymer, 30 parts or less hydrophobic polymer for every 1 part hybrid polymer, 20 parts or less hydrophobic polymer for every 1 part hybrid polymer, 19 parts or less hydrophobic polymer for every 1 part hybrid polymer, 19 parts or less hydrophobic polymer for every 1 part hybrid polymer, 17 parts or less hydrophobic polymer for every 1 part hybrid polymer, 16 parts or less hydrophobic polymer for every 1 part hybrid polymer, 15 parts or less hydrophobic polymer for every 1 part hybrid polymer, 14 parts or less hydrophobic polymer for every 1 part hybrid polymer, 13 parts or less hydrophobic polymer for every 1 part hybrid polymer, 12 parts or less hydrophobic polymer for every 1 part hybrid polymer, 11 parts or less hydrophobic polymer for every 1 part hybrid polymer, 10 parts or less hydrophobic polymer for every 1 part hybrid polymer, 9 parts or less hydrophobic polymer for every 1 part hybrid polymer, 8 parts or less hydrophobic polymer for every 1 part hybrid polymer, 7 parts or less hydrophobic polymer for every 1 part hybrid polymer, 6 parts or less hydrophobic polymer for every 1 part hybrid polymer, 5 parts or less hydrophobic polymer for every 1 part hybrid polymer, 4 parts or less hydrophobic polymer for every 1 part hybrid polymer, 3 parts or less hydrophobic polymer for every 1 part hybrid polymer, 2 parts or less hydrophobic polymer for every 1 part hybrid polymer, 0.9 parts or less hydrophobic polymer for every 1 part hybrid polymer, 0.8 parts or less hydrophobic polymer for every 1 part hybrid polymer, 0.7 parts or less hydrophobic polymer for every 1 part hybrid polymer, 0.6 parts or less hydrophobic polymer for every 1 part hybrid polymer, 0.5 parts or less hydrophobic polymer for every 1 part hybrid polymer, 0.4 parts or less hydrophobic polymer for every 1 part hybrid polymer, 0.3 parts or less hydrophobic polymer for every 1 part hybrid polymer, 0.2 parts or less hydrophobic polymer for every 1 part hybrid polymer, 0.1 parts or less hydrophobic polymer for every 1 part hybrid polymer, 0.09 parts or less hydrophobic polymer for every 1 part hybrid polymer, 0.08 parts or less hydrophobic polymer for every 1 part hybrid polymer, 0.07 parts or less hydrophobic polymer for every 1 part hybrid polymer, 0.06 parts or less hydrophobic polymer for every 1 part hybrid polymer, 0.05 parts or less hydrophobic polymer for every 1 part hybrid polymer, 0.04 parts or less hydrophobic polymer for every 1 part hybrid polymer 0.03, parts or less hydrophobic polymer for every 1 part hybrid polymer, or 0.02 parts or less hydrophobic polymer for every 1 part hybrid polymer.

In one or more embodiments, the weight or mole ratio of a hydrophobic polymer to a hybrid polymer in a polyplex transfection complex can be 20 parts hydrophobic polymer for every 1 part hybrid polymer, 10 parts hydrophobic polymer for every 1 part hybrid polymer, 5 parts hydrophobic polymer for every 1 part hybrid polymer, 3 parts hydrophobic polymer for every 1 part hybrid polymer, or 1 part hydrophobic polymer for every 1 part hybrid polymer.

In one or more embodiments, a polyplex transfection complex includes a hybrid polymer of the present disclosure and does not include a hydrophobic polymer of the present disclosure.

The ratio of the amine group (e.g., quinuclidine nitrogen) of the polymers (the hybrid polymer and/or hydrophobic polymer) to the phosphate groups of the oligonucleotide (N/P ratio) may affect the efficiency of the polyplex as a transfection agent complex. As used herein, the N/P ratio refers to the N/P ratio of a single polyplex or the average N/P ratio of a plurality of polyplexes. In one or more embodiments the polyplex N/P ratio is 1 or greater, 3 or greater, 5 or greater, 6 or greater, 8 or greater, 10 or greater, 12 or greater, 14 or greater, 16 or greater, 18 or greater, 20 or greater, 25 or greater, or 30 or greater. In one or more embodiments the polyplex N/P ratio is 40 or less, 35 or less, 30 or less, 25 or less, 20 or less, 18 or less, 16 or less, 14 or less, 12 or less, 10 or less, 8 or less, 6 or less, 5 or less, or 3 or less. In one or more embodiments the polyplex N/P ratio is 1 to 40, 1 to 35, 1 to 30, 1 to 25, 1 to 20, 1 to 18, 1 to 16, 1 to 14, 1 to 12, 1 to 12, 1 to 10, 1 to 8, 1 to 6, 1 to 5, or 1 to 3. In one or more embodiments the polyplex N/P ratio is 3 to 40, 3 to 35, 3 to 30, 3 to 25, 3 to 20, 3 to 18, 3 to 16, 3 to 14, 3 to 12, 3 to 12, 3 to 10, 3 to 8, 3 to 6, or 3 to 5. In one or more embodiments the polyplex N/P ratio is 5 to 40, 5 to 35, 5 to 30, 5 to 25, 5 to 20, 5 to 18, 5 to 16, 5 to 14, 5 to 12, 5 to 12, 5 to 10, 5 to 8, or 5 to 6. In one or more embodiments the polyplex N/P ratio is 6 to 40, 6 to 36, 6 to 30, 6 to 26, 6 to 20, 6 to 18, 6 to 16, 6 to 14, 6 to 12, 6 to 12, 6 to 10, or 6 to 8. In one or more embodiments the polyplex N/P ratio is 8 to 40, 8 to 36, 8 to 30, 8 to 26, 8 to 20, 8 to 18, 8 to 16, 8 to 14, 8 to 12, 8 to 12, or 8 to 10. In one or more embodiments the polyplex N/P ratio is 10 to 40, 10 to 36, 10 to 30, 10 to 26, 10 to 20, 10 to 18, 10 to 16, 10 to 14, or 10 to 12. In one or more embodiments the polyplex N/P ratio is 12 to 40, 12 to 36, 12 to 30, 12 to 26, 12 to 20, 12 to 18, 12 to 16, or 12 to 14. In one or more embodiments the polyplex N/P ratio is 14 to 40, 14 to 36, 14 to 30, 14 to 26, 14 to 20, 14 to 18, or 14 to 16. In one or more embodiments the polyplex N/P ratio is 16 to 40, 16 to 36, 16 to 30, 16 to 26, 16 to 20, or 16 to 18. In one or more embodiments the polyplex N/P ratio is 18 to 40, 18 to 36, 18 to 30, 18 to 26, or 18 to 20. In one or more embodiments the polyplex N/P ratio is 20 to 40, 20 to 36, 20 to 30, or 20 to 26. In one or more embodiments the polyplex N/P ratio is 25 to 40, 25 to 36, 25 to 30, 30 to 36, or 35 to 40.

A polyplex includes an oligonucleotide. As used herein, the term “oligonucleotide” refers to a polymer of two or more nucleotides. An oligonucleotide may be single-stranded; that is, include a single polymer of nucleotides, or double-stranded; that is, include two polymers of nucleotides that are at least partially hybridized to each other. An oligonucleotide may include deoxyribonucleotides, ribonucleotides, or both. The oligonucleotide may include non-canonical deoxyribonucleotides, non-canonical ribonucleotides, or both. Examples of oligonucleotides that may be transfected into cells using the copolymers of the present disclosure include but are not limited to, plasmid DNA (pDNA), messenger RNA (mRNA), antisense oligonucleotides, small interfering RNA, micro-RNA, guide RNA, Cas9-sgRNa complexes, aptamers, derivatives thereof, and combinations thereof. In one or more embodiments, the hybrid polymer of the present disclosure may be used as a transfection agent for the transfection of pDNA into a cell.

The size of the polyplex may affect the transfection efficiency of the polyplex. For example, the size of the polyplex may influence the internalization pathway used by the cell during transfection. As used herein, the size of a polyplex transfection complex refers to the hydrodynamic diameter (dh) of a polyplex or the average hydrodynamic diameter of a plurality of polyplexes. The hydrodynamic diameter may be measured using dynamic light scattering, for example. A polyplex (or plurality of polyplexes) may have an undiluted dh and a treatment dh. The undiluted dh refers to the dh of a polyplex (or a plurality of polyplexes) in the mixture in which they are formed prior to any dilution.

In one or more embodiments, the medium in which polyplexes are initially formed may be unsuitable for live cells. For example, polyplexes may be formed in a solution at an acidic pH (e.g., a pH of 1 to 5). Direct exposure of the cells to such as solution may stress and/or kill the cells. Polyplexes may also be formed in pure water without additives such as salts. Such solutions can rupture the cells due to a difference in osmotic pressure inside and outside the cells. Additionally, polyplexes may be formed using a high concentration of the copolymer and/or oligonucleotide. The resultant polyplex solution may include a concentration of polyplexes that is too high for exposure to cells. As such, in some embodiments, prior to exposure to cells, an undiluted polyplex mixture is diluted with a dilution medium. The dilution factor, the dilution medium, and the pH of the medium are chosen so as to reduce cell death upon exposure to the polyplexes. In one or more embodiments, when a mixture of polyplexes is diluted, the polyplexes may aggregate to form complexes having a larger dₕ than prior to dilution. As used herein, the treatment dₕ also referred to as an aggregate dₕ, refers to the dₕ of the polyplex (or plurality of polyplexes) that are used to treat (e.g., transfect cells). In one or more embodiments, the undiluted dₕ and the treatment dₕ are the same. In other embodiments, the undiluted dₕ and the treatment dₕ are different. In some such embodiments, the undiluted dₕ is smaller than the treatment dₕ. Polyplexes that have been diluted and are ready for exposure to cells may be referred to as diluted polyplexes or aggregated polyplexes.

In one or more embodiments, the undiluted dₕ and/or the treatment dₕ is 20 nm or greater, 40 nm or greater, 50 nm or greater, 60 nm or greater, 70 nm or greater, 80 nm or greater, 90 nm or greater, 100 nm or greater, 200 nm or greater, 300 nm or greater, 400 nm or greater, 500 nm or greater, 600 nm or greater, 700 nm or greater, 800 nm or greater, 900 nm or greater, 1000 nm or greater, 1100 nm or greater, 1200 nm or greater, 1300 nm or greater, 1400 nm or greater, 1500 nm or greater, 1600 nm or greater, 1700 nm or greater, 1800 nm or greater, or 1900 nm or greater. In one or more embodiments, the undiluted dₕ and/or the treatment dₕ is 2000 nm or less, 1900 nm or less, 1800 nm or less, 1700 nm or less, 1600 nm or less, 1500 nm or less, 1400 nm or less, 1300 nm or less, 1200 nm or less, 1100 nm or less, 1000 nm or less, 900 nm or less, 800 nm or less, 700 nm or less, 600 nm or less, 500 nm or less, 400 nm or less 300 nm or less, 200 nm or less, 100 nm or less, 90 nm or less, 80 nm or less, 70 nm or less, 60 nm or less, 50 nm or less, or 40 nm or less. In one or more embodiments, the undiluted dₕ and/or the treatment dₕ is 20 nm to 2000 nm, 20 nm to 1900 nm, 20 nm to 1800 nm, 20 nm to 1700 nm, 20 nm to 1600 nm, 20 nm to 1500 nm, 20 nm to 1400 nm, 20 nm to 1300 nm, 20 nm to 1200 nm, 20 nm to 1100 nm, 20 nm to 1000 nm, 20 nm to 900 nm, 20 nm to 800 nm, 20 nm to 700 nm, 20 nm to 600 nm, 20 nm to 500 nm, 20 nm to 400 nm, 20 nm to 300 nm, 20 nm to 200 nm, 20 nm to 100 nm, 20 nm to 90 nm, 20 nm to 80 nm, 20 nm to 60 nm, or 20 nm to 50 nm. In one or more embodiments, the undiluted dₕ and/or the treatment dₕ is 20 nm to 200 nm, 20 nm to 100 nm, 40 nm to 200 nm, 40 nm to 100 nm, 40 nm to 80 nm, 40 nm to 60 nm, 100 nm to 1500 nm or greater, 500 nm to 1500 nm or greater, 1000 nm to 1500 nm or greater, 300 nm to 900 nm, 300 nm to 800 nm, 400 nm to 800 nm, or 400 nm to 600 nm.

In another embodiment, the present disclosure describes a transfection composition. The transfection composition includes a polyplex transfection complex of the present disclosure. The transfection complex includes an oligonucleotide and a hybrid polymer of the present disclosure, a hydrophobic polymer of the present disclosure, or both. The transfection composition further includes a transfection medium. The size of the polyplex or plurality of polyplexes in a transfection composition is the treatment dₕ (as described elsewhere herein). The treatment dₕ may be any treatment dₕ as described herein.

The transfection medium may be any suitable medium for contact with cells. In one or more embodiments, the medium comprises water. In one or more embodiments, the medium includes salts and or agents or compounds that promote cell health and/or growth. In one or more embodiments, the medium is a cell growth medium, such as, for example, Dulbecco's Modified Eagle Medium (DMEM), Minimum Essential Medium (MEM), RPMI 1640 Medium, Opti-MEM™ I. In one or more embodiments, the medium includes proteins. In other embodiments, the medium does not include proteins. In one or more embodiments, the medium is serum-free. In other embodiments, the medium includes serum.

The pH of the transfection composition may vary. In one or more embodiments, the pH of the medium is 2 or greater, 3 or greater, 4 or greater, 5 or greater, 6 or greater, 7 or greater, or 8 or greater. In one or more embodiments, the pH of the medium is 9 or less, 8 or less, 7 or less, 6 or less, 5 or less, 4 or less, or 3 or less. In some embodiments, the pH of the medium is 4 to 8, 5 to 8, 6 to 8, or 7 to 8.

In another embodiment, the present disclosure describes a method. The method includes forming a polyplex transfection complex. The method generally includes mixing a hybrid polymer and/or a hydrophobic polymer with the oligonucleotide to create a first mixture. The method also includes incubating the first mixture for a period of time to form a second mixture comprising the polyplex transfection complex. In some embodiments, the second mixture is a transfection composition that can be used to treat cells.

The concentration of the hybrid polymer and/or the concentration of the hydrophobic polymer (if present), and the concentration of the oligonucleotide in the first mixture may vary depending at least in part on the desired N/P ratio, the identity and properties of the quinoline-containing polymer, the identity and properties of the oligonucleotide, the number of cells to be treated, other factors, or any combination thereof.

In one or more embodiments, the first mixture includes water. In one or more embodiments, the first mixture includes an aqueous solution of water and an acid. The acid is used to adjust the pH to a value of 1 to 5. A low pH may be beneficial to polyplex formation as a low pH may increase the probability that the amines of the polymer are protonated. Protonation of the amines gives the amines a positive charge which may increase electrostatic interactions between the polymer and the negatively charged backbone of the oligonucleotide. Any suitable acid or buffer may be included. Examples of acids include but are not limited to, citric acid, acetic acid, hydrochloric acid, formic acid, lactic acid, uric acid, malic acid, tartaric acid, sulfuric acid, and the like. Examples of buffers include sodium acetate buffer, chloroacetate buffer, and the like.

The length of the first incubation period of time may affect the transfection efficiency of the polyplex. In one or more embodiments, the first period of time is 1 minute or greater, 15 minutes or greater, 20 minutes or greater, 25 minutes or greater, 30 minutes or greater, 40 min or greater, 1 hour or greater, or 5 hours or greater. In one or more embodiments, the first period of time is 5 hours or less, 1 hour or less, 40 minutes or less, 35 minutes or less, 30 minutes or less, 25 minutes or less, 20 minutes or less, or 15 minutes or less. In one or more embodiments, the first period of time is 15 minutes to 40 minutes, 15 minutes to 35 minutes, 15 minutes to 30 minutes, 15 minutes to 25 minutes, or 15 minutes to 20 minutes. In one or more embodiments, the first period of time is 20 minutes to 40 minutes, 20 minutes to 35 minutes, 20 minutes to 30 minutes, or 20 minutes to 25 minutes. In one or more embodiments, the first period of time is 25 minutes to 40 minutes, 25 minutes to 35 minutes, or 25 minutes to 30 minutes. In one or more embodiments, the first period of time is 30 minutes to 40 minutes or 30 minutes to 35 minutes. In one or more embodiments, the first period of time is 25 minutes to 35 minutes.

In one or more embodiments, the method further includes diluting the second mixture with a dilution medium to create a third mixture. In some embodiments, the third mixture is a transfection composition that can be used to treat cells. In such embodiments, the dilution medium is a transfection medium. In one or more embodiments, the method further includes incubating the third mixture for second period of time to form a transfection composition. In some such embodiments, the incubation of the third mixture allows for the aggregation of polyplexes to form polyplex particles having a larger dₕ than in the second mixture.

The second mixture may be diluted with an amount of a medium such that the concentration of the polyplexes in the third mixture is 99% or less, 90% or less, 80% or less, 70% or less, 60% or less, 50% or less, 40% or less, 30% or less, 20% or less, 10% or less, 5% or less, 1% or less 0.1% or less, 0.01% or less, 0.001% or less, 0.0001% or less of the concentration of the polyplexes in the second mixture. In one or more embodiments, the second mixture may be diluted with an amount of a medium such that the concentration of the polyplexes in the third mixture is 0.0001% or greater, 0.001% or greater, 0.01% or greater, 0.1% or greater, 1% or greater, 5% or greater, 10% or greater, 20% or greater, 30% or greater, 40% or greater, 50% or greater, 60% or greater, 70% or greater, 80% or greater, or 90% or greater of the concentration of the polyplexes in the second mixture. In one or more embodiments, the second mixture may be diluted with an amount of a medium such that the concentration of the polyplexes is 10% to 90%, 20% to 80%, or 40% to 60% of the concentration of the polyplexes in the second mixture.

The length of the second period of time may affect the efficiency of transfection. In one or more embodiments, the second period of time is 1 min or greater, 15 minutes or greater, 20 minutes or greater, 25 minutes or greater, or 30 minutes or greater, 40 minutes or greater, 1 hour or greater, or 5 hours or greater. In one or more embodiments, the second period of time is 5 hours or less, 1 hour or less, 40 minutes or less, 35 minutes or less, 30 minutes or less, 25 minutes or less, 20 minutes or less, or 15 minutes or less. In one or more embodiments, the second period of time is 15 minutes to 40 minutes, 15 minutes to 35 minutes, 15 minutes to 30 minutes, 15 minutes to 25 minutes, or 15 minutes to 20 minutes. In one or more embodiments, the second period of time is 20 minutes to 40 minutes, 20 minutes to 35 minutes, 20 minutes to 30 minutes, or 20 minutes to 25 minutes. In one or more embodiments, the second period of time is 25 minutes to 40 minutes, 25 minutes to 35 minutes, or 25 minutes to 30 minutes. In one or more embodiments, the second period of time is 30 minutes to 40 minutes or 30 minutes to 35 minutes. In one or more embodiments, the second period of time is 25 minutes to 35 minutes.

In another aspect, the present disclosure describes a method of transfecting a cell with a polyplex transfection complex of the present disclosure. In some such embodiments, the polyplex transfection complex is in a transfection composition of the present disclosure. The polyplex transfection complex and/or transfection composition may include any component as disclosed herein and may be formed according to any method disclosed herein. The method includes contacting a polyplex transfection complex or transfection composition with a cell to form a transfection mixture. In one or more embodiments, the transfection composition is the third mixture following the second incubation time from the method of forming the polyplex transfection complex described herein. In one or more embodiments, the method further incudes incubating the transfection mixture for a transfection time. In one or more embodiments, the method further includes quenching the transfection mixture.

The pH of the polyplex transfection complex or transfection composition used to treat the cells may affect the efficiency of transfection. For example, a pH that is too high or too low may kill the cells. In one or more embodiments, the pH of the polyplex or transfection composition is between 5 and 8. In one or more embodiments, the pH of polyplex or transfection composition is between 7 and 8.

Similarly, the length of the transfection time may affect the efficiency of transfection. In one or more embodiments, the transfection time is 1 minute or greater, 15 minutes or greater, 20 minutes of greater, 25 minutes or greater, or 30 minutes or greater, 40 minutes or greater, 60 minutes or greater, 120 minutes or greater, 5 hours or greater, 12 hours or greater, 24 hours or greater, 48 hours or greater, 64 hours or greater, or 96 hours or greater. In one or more embodiments, the transfection time of time is 96 hours or less, 64 hours or less, 48 hours or less, 24 hours or less, 12 hours or less, 5 hours or less, 120 minutes or less, 60 minutes or less, 40 minutes of less, 35 minutes or less, 30 minutes or less, 25 minutes or less, or 20 minutes or less. In one or more embodiments, the transfection time is 15 minutes to 120 minutes, 15 minutes to 100 minutes, 15 minutes to 80 minutes, 15 minutes to 60 minutes, 15 minutes to 40 minutes, 15 minutes to 35 minutes, 15 minutes to 30 minutes, 15 minutes to 25 minutes, or 15 minutes to 20 minutes. In one or more embodiments, transfection time is 20 minutes to 120 minutes, 20 minutes to 100 minutes, 20 minutes to 80 minutes, 20 minutes to 60 minutes, 20 minutes to 40 minutes, 20 minutes to 35 minutes, 20 minutes to 30 minutes, or 20 minutes to 25 minutes. In one or more embodiments, the transfection time is 25 minutes to 120 minutes, 25 minutes to 100 minutes, 25 minutes to 80 minutes, 25 minutes to 60 minutes, 25 minutes to 40 minutes, 25 minutes to 35 minutes, or 25 minutes to 30 minutes. In one or more embodiments, the transfection time is 30 minutes to 120 minutes, 30 minutes to 100 minutes, 30 minutes to 80 minutes, 30 minutes to 60 minutes, 30 minutes to 40 minutes or 30 minutes to 35 minutes. In one or more embodiments, the transfection time is 25 minutes to 35 minutes.

Quenching the transfection mixture decreases the likelihood of transfection. Quenching the transfection mixture may increases cell survival during and/or after the transfection process. In one or more embodiments, quenching the transfection mixture includes adding serum-containing media to the transfection mixture to create a quenched mixture. The serum in the serum-containing media disrupts the ability of the transfection complex to be transfected into a cell.

In an embodiment, the present disclosure describes a transfection kit. The kit includes a transfection reagent of the present disclosure. The transfection reagent may be provided in a solution. In one or more embodiments, the transfection reagent may be provided in an acidic solution having a pH of 1 to 5. In one or more embodiments, the kit may include a serum free medium. In one or more embodiments, the kit may include a serum-containing medium. In one or more embodiments, the kit may include ultrapure water. In one or more embodiments, the kit may include instructions.

In the description and claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

As used herein, “alkyl” or “alkyl group” refers to a fully saturated, straight, or branched hydrocarbon chain having from one to twelve carbon atoms, and which is attached to the rest of the molecule by a single bond.

As used herein, “alkene” refers to carbon-carbon double bond. An alkene is a functional group that may be a part of a larger molecule. An alkene may be a polymerizable functional group.

When a group is present more than once in a formula described herein, each group is independently selected, whether specifically stated or not. For example, when more than one Y group is present in a formula, each Y group is independently selected. Furthermore, subgroups contained within these groups are also independently selected. For example, when each Y group contains an R1, each R1 is also independently selected.

As used herein, the symbols

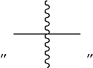

and “-” (hereinafter can be referred to as “a point of attachment bond or point of attachment”) when used in the context of a compound, denote a bond that is a point of attachment between two chemical entities, one of which is depicted as being attached to the point of attachment bond and the other of which is not depicted as being attached to the point of attachment bond. For example,

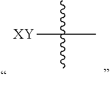

and —XY” indicate that the chemical entity “XY” is bonded to another chemical entity via the point of attachment bond.

The point of attachment of the chemical group to the compound may be described in several ways. For example, in some embodiments, the organic group may be described as the monovalent or radical of the respective functional group (e.g., alkyl for alkane, alkenyl for an alkene containing group). In some embodiments, where a general formula is shown with a covalent bond connecting the chemical group to a compound, the chemical group may be described as the common functional group that is or is contained within the chemical group. For example, if the chemical group R is described relative to the formula CH₃CH₂CH₂—R, the chemical group may be described, for example, as an alkene.

The terms “alkenyl” and “alkenyl group” refers to a univalent group that is a radical of an alkene and includes groups that are linear, branched, cyclic, or combinations thereof. An alkenyl group has one or more double bonds. The location of the double bond may be anywhere along the alkenyl. The radical may be a part of the double bond (e.g., —CHCH—R). The radical may be a part of a single bond (e.g., —CH₂—R).

As used herein, the terms “formed from” and “polymerized from” are open ended and may include other components that may not be expressly described relative to the subject that is formed from or polymerized from the stated components. For example, a polymer formed from or polymerized from a quinoline-containing monomer may include capping groups or other groups not expressly mentioned.

### EXAMPLES

The present disclosure is illustrated by the following examples. It is to be understood that the particular examples, materials, amounts, and procedures are to be interpreted broadly in accordance with the scope and spirit of the invention as set forth herein.

### Example 1

Example 1 explores the properties and characteristics of polyplexes that include pDNA, a hybrid polymer, and a hydrophobic polymer. The hybrid polymer included a hydrophobic block polymerized from 2-hydroxyethyl acrylate (HEA) and a hydroquinine monomer (HQ) as well as a polyethylene glycol (PEG) hydrophilic block (abbreviated as PHQ polymer, or simply PHQ). The hydrophobic polymer was polymerized from 2-hydroxyethyl acrylate (HEA) and the same hydroquinine monomer as used in the hybrid polymer (abbreviated as HQ polymer, or simply HQ).

For polyplex formation, 25 polymer formulations were tested, each formulation including one hybrid polymer and one hydrophobic polymer. In the formulation series, the PHQ polymer was kept constant while the HQ polymers varied by hydroquinine content. More specifically, HQ polymers were synthesized that had 25 mol-% (HQ-25), 35 mol-% (HQ-35), 44 mol-% (HQ-44), 60 mol-% (HQ-60), and 100 mol-% (HQ-100) hydroquinine content.

Binary mixtures of PHQ and a HQ-X (where X is the mol-% of hydroquinine in the polymer) were formulated based on the contribution of each polymer to the overall N/P ratio, or the molar ratio of quinuclidine nitrogen atoms to the phosphate groups in the mRNA. For example, an N/P of 16 indicates there are 16 times more quinuclidine moieties than phosphate groups in the formulation. A formulation having 10% PHQ and 90% HQ-X indicates that the contribution from HQ-X in this mixture is 14.4 and the contribution from the hybrid polymer (PHQ) is 1.6. Each of HQ-25, HQ-35, HQ-44, HQ-60, and HQ-100 where separately formulated with 5%, 10%, 20%, 50%, or 75% PHQ. Additionally, a formulation of 100% PHQ was tested.

### Materials:

Synthesis of Polymers: Quinine (anhydrous, 99% total base with ≤5% dihydroquinine) was bought from Alfa Assar (Tewksbury, MA). Hydroquinine (98%), 2-hydroxyethyl acrylate (HEA), 2-hydroxyethyl methacrylate (HEMA), 4-cyano-4-[(dodecylsulfanylthiocarbonyl)sulfanyl]pentanoic acid (CDP), 2-(dodecylthiocarbonothioylthio)-2-methylpropionic acid (DDMAT), azobisisobutyronitrile (AIBN), 4,4′-azobis(cyanovaleric acid) (V-501), N,N′-dicyclohexylcarbodiimide (DCC), silica gel (technical grade, pore size 60 Å, 70-230 mesh) were purchased from Sigma-Aldrich (St. Louis, MO). 2-Isocyanatoethyl acrylate (stabilized with BHT) was purchased from TCI Chemicals (Portland, Oregon). mPEG-OH, 10 kDa, was purchased from Creative PEGWorks (Chapel Hill, NC). 4-Dimethylaminopyridine (DMAP) was purchased from Acros Organics. Spectra/Por™ Pre-wetted RC dialysis tubing (MW cutoff˜1 kDa) was purchased from Spectrum Chemical Mfg. Corp (New Brunswick, NJ). The tubing was soaked in and rinsed with Milli-Q water prior to use.

Transfection: GFP expressing pZsGreenl-N1 plasmid DNA (4.7 kbp) was purchased from Aldevron (Fargo, ND). CCK-8 cell counting kit was purchased from Bimake (Houston, TX). CELLTRACE™ Calcein Violet, QUANT-IT™ PicoGreen were purchased from ThermoFisher Scientific (Waltham, MA). CellScrub buffer was purchased from Genlantis (San Diego, CA. JETPEI® was purchased from Polyplus-transfection (New York, NY). Label IT Nucleic Acid Labeling Kit, Cy5 was purchased from Mirus Bio (Madison, WI).

Cell Culture: Dulbecco's modified eagle medium (DMEM; high glucose, pyruvate, and GlutaMAX supplemented), Opti-MEM I reduced serum medium, Trypsin-EDTA (0.05%), Phosphate buffered saline (PBS) pH=7.4, UltraPure DNAse/RNAse-Free distilled water (DI H2O), Antibiotic-antimycotic (100×) were purchased from Life Technologies, ThermoFisher Scientific (Carlsbad, CA). Heat inactivated fetal bovine serum (HI FBS) was purchased from Corning Life Sciences (Durham, NC). Human embryonic kidney cell line (HEK293T) engineered to have the traffic light reporter (TLR) system were from the laboratory of Mark Osborne at the University of Minnesota. The subclones were made at the Genome Engineering Shared Resource (Minneapolis, MN) to obtain a stable cell line.

Instrument Details: NMR spectra were recorded on AX-400 Bruker Avance III HD (Billerica, MA). Mass spectra were recorded on BioTOF II ESI-TOF Mass Spectrometer. Size exclusion chromatography was performed on Agilent INFINITY 1260 HPLC system equipped with Wyatt DAWN Heleos II multiangle laser light scattering detector and Wyatt OPTILAB T-rEX refractive index detector. Molar masses were calculated using dn/dc values calculated from the refractive index signal using samples with known concentration with an assumption of 100% mass recovery. Absorbance and fluorescence measurements of polymers and polyplexes were acquired using Synergy H1 multimode plate reader (BioTek; Winooski, VT). pH measurements and potentiometric titrations were done with OrionStar T910 (ThermoFisher Scientific; Waltham, MA). Flow cytometry was performed on ZE5 cell analyzer (Bio-Rad; Hercules, CA) and results were analyzed with analyzed using FlowJo software (Ashland, OR). Dynamic light scattering (DLS) measurements were made with a Zetasizer Nano ZS (Malvern; Worcestershire, UK) with a 4.0 mW He—Ne laser (λ=633 nm) and DYNAPRO® Plate Reader III (Malvern; Worcestershire, UK) (λ=820 nm, detector angle=150°). Cell suspensions were counted with a Countess II automated cell counter (ThermoFisher Scientific; Waltham, MA) with dead cell discrimination by dilution (1:1) with trypan blue (0.4%). Widefield fluorescence microscopy was carried out using an EVOS Digital Microscope (AMG Life Technologies; Grand Island, NY). All statistical calculations were performed with GraphPad Prism v9.4.1.

### Methods:

Cell Culture: The engineered HEK293T cells were cultured in DMEM supplemented with 10% fetal bovine serum and 1% antibiotic-antimycotic at 37° C. and 5% CO2 in 75 cm² and 175 cm² cell culture flasks respectively.

Transfection Protocol: Cells were plated in 48-well plates at the density of 55,000 cells/mL, 24 hours before transfection. Manufacturer's protocol was used for transfection with JETPEI and LIPOFECTAMINE 2000. Previously reported transfection protocol was used with minor modifications (Van Bruggen, C.; Punihaole, D.; Keith, A. R.; Schmitz, A. J.; Tolar, J.; Frontiera, R. R.; Reineke, T. M. Quinine Copolymer Reporters Promote Efficient Intracellular DNA Delivery and Illuminate a Protein-Induced Unpackaging Mechanism. Proc. Natl. Acad. Sci. U.S.A 2020, 117, 32919-32928). Secondary solutions of polymer and pDNA were mixed in 1:1 volume ratio and incubated for 30 minutes at room temperature to form primary polyplexes at desired N/P ratios. The polyplex solutions were then diluted in 1:2 volume ratio using Opti-MEM to mature the polyplexes. After 30 minutes, the cells were washed with phosphate buffered saline (PBS) and treated with the mature polyplex solution with each well receiving 0.5 microgram of pDNA. The cells were then incubated at 37° C. with 5% CO2 for 30 minutes before addition of 0.5 mL of serum-supplemented DMEM into each well. Fresh supplemented media was added to each well 24 hours later. Negative controls were performed with cells being similarly treated with media that is equivalently diluted but does not contain any polymer and pDNA to confirm that the cells are tolerant of the media changes, the wash procedures, and the duration of transfection. To transfect the cells, aggregated polyplexes were prepared according to the regular transfection protocol followed by addition of polyplex solution directly into serum supplemented media.

### Synthesis of HQ

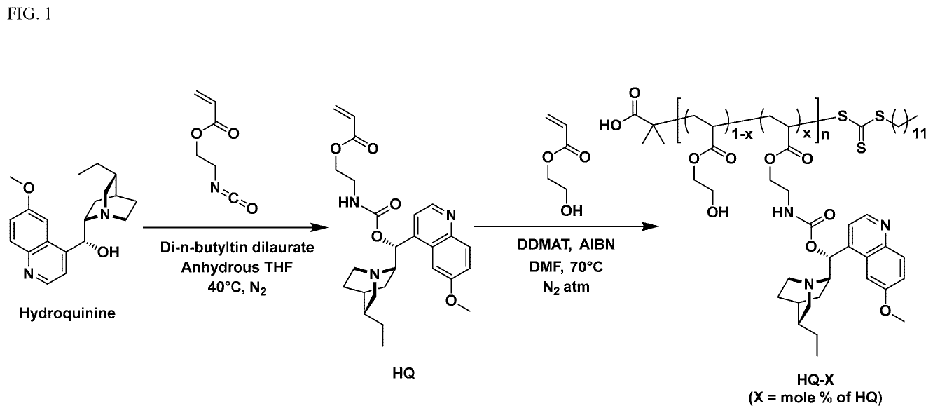

FIG. 1 shows the synthetic scheme for synthesis of the hydroquinine monomer (HQ) and copolymerization of the hydroquinine monomer with 2-hydroxyethyl acrylate (HEA) using reversible addition fragmentation chain transfer (RAFT). Polymers made via this scheme are referred to as HQ-X polymers where X indicates the mol-% of the hydroquinine monomer in the polymer.

Synthesis of the hydroquinine monomer: In a round bottom flask, hydroquinine (4.0 g, 12.3 mmol) was dissolved in anhydrous tetrahydrofuran (TIF; 50 mL) followed by the addition of dibutyltin dilaurate (DBTDL) (23 mg, 0.036 mmol dissolved in 5 mL anhydrous TIF) into the reaction mixture. A solution of 2-isocyanatoethyl acrylate (2.3 g, 16 mmol) in anhydrous THE (20 mL) was added dropwise to the reaction mixture under continuous stirring. After stirring for 30 minutes at room temperature, the reaction mixture was heated to 40° C. in an oil bath and was stirred for another 24 hours under N2 atmosphere. Completion of the reaction was confirmed via TLC, and excess 2-isocyanatoethyl acrylate was quenched by adding distilled water. The quenched reaction mixture was washed with ethyl acetate (3 times with 200 mL) to extract the product into the organic phase. The organic phase was further washed with brine, followed by drying over anhydrous Na₂SO₄, and then was concentrated under vacuum. The pure product was isolated by flash chromatography using silica gel as the stationary phase and 20% methanol in dichloromethane (DCM) as the mobile phase. The purified product was dried under a high vacuum overnight before further use.

Polymerization of the hydroquinine monomer via Reversible Addition-Fragmentation Chain-Transfer Polymerization (RAFT): Into one-dram vials, monomers (HQ and HEA), chain transfer agent (DDMAT), and initiator (AIBN) were transferred and dissolved in anhydrous dimethylformamide (DMF; total monomer concentration=1 M, [Total monomer]:[DDMAT]:[AIBN]=200:1:0.2). The vials were closed with SUBA-SEAL septa and the reaction mixture was purged with N2 for at least 30 minutes. Subsequently, the vials were transferred to a metal heating block and stirred vigorously for six hours at 80° C. The reactions were quenched by rapidly cooling the reaction mixture in a liquid N₂ bath followed by exposure to atmospheric oxygen. The reaction mixture was then diluted with 10% THE (inhibitor-free) in methanol and then transferred to RC dialysis tubing and then dialyzed for four days using 10% THE (inhibitor-free) in methanol. The purified polymer solutions were first concentrated and then dried under a high vacuum overnight to yield the pure polymer. For all experiments and assays, polymer stock solutions were prepared in 3% acetic acid in water solution. The polymer solutions were vortexed well and then filtered using a 0.22-micrometer syringe filter before use.

A family of five polymers (HQ-X, X=mole percentage of hydroquinine), with mole percentages of hydroquinine ranging from 25% to 100% were prepared.

### Synthesis of PHQ

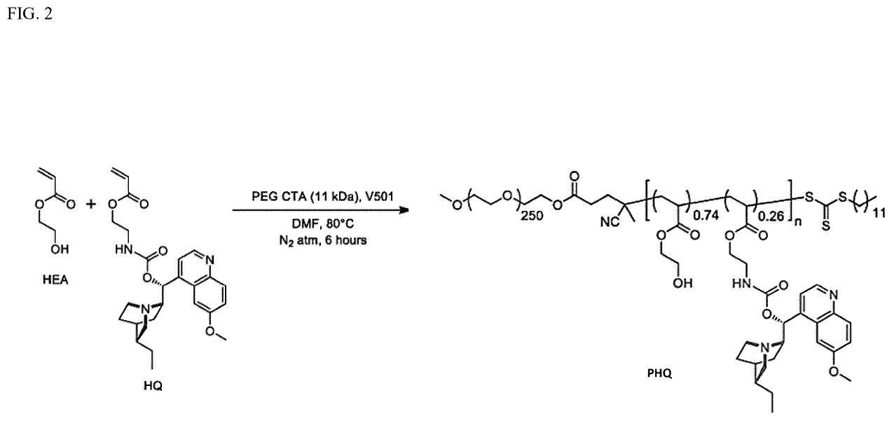

FIG. 2 shows the synthetic scheme for synthesizing a from 2-hydroxyethyl acrylate (HEA) and a hydroquinine monomer (HQ). The resultant polymer is referred to as PHQ. The chain transfer agent (CTA) used in the synthesis of the hybrid polymers was poly(ethylene glycol) 4-cyano-4-(phenylcarbonothioylthio)pentanoate (PEG-CTA) with V-501 as the initiator. Monomers, PEG-CTA, initiator, and solvent (DMF) were added to a reaction vial and sparged with nitrogen gas for 45 minutes. The reaction vial was then put on the heat/stirring to react at 80° C. for six hours. The polymerization was quenched by freezing with liquid nitrogen and exposing the reaction to atmospheric oxygen. For these reactions, the ratio of initiator to CTA was 0.05:1 to maintain a large fraction of living chains for eventual chain extension. For the chain extension polymerizations needed to make the hybrid polymers with carbohydrates in the hydrophilic block, the same procedure was applied with some slight modifications. Instead of PEG-CTA as the chain transfer agent, carbohydrate blocks were used as macro-CTAs. Also, the initiator:macro-CTA ratio for these chain extensions was 0.1:1. The reactions were run for 24 hours.

All polymers were purified using dialysis in pre-soaked regenerated cellulose dialysis tubing with a molecular weight cutoff of 1000 Da. The solvent used for dialysis was 75/25 (v/v) methanol/TIF (inhibitor free). Dialysis was run for four days with a solvent change once per day. Purified polymers were dried under vacuum to obtain pale yellow to white colored powders.

### Preparation of Polyplexes

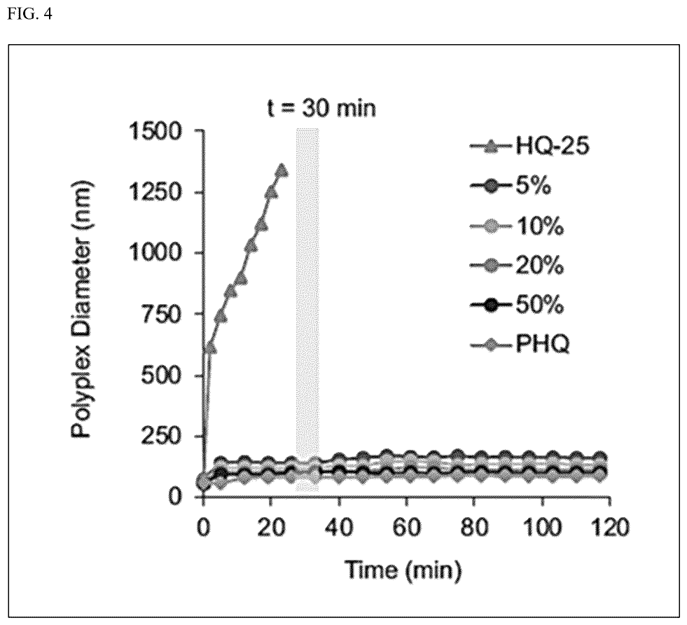

For preparing the polyplexes, first polymer stocks were prepared by dissolving polymers in 3% acetic acid by vigorous vortexing at 3000 rpm for 15 minutes followed by sterile filtration using 0.22 μm PDVF-based syringe filter and stored in −20° C. The polymer stock solutions were diluted in a way such that the solutions had a concentration of 0.0104 M for the quinuclidine nitrogen atoms which was also used in N/P calculation. For formulating the polyplexes, both pDNA and polymer stocks were diluted to appropriate concentrations using water. pDNA was diluted to 20 μg/mL and polymer stocks were diluted based on the desired N/P ratio. The diluted polymer solution was added to the diluted pDNA solution and mixed thoroughly. After that, the second polymer stock solution was added to the polyplex solution. Polyplexes were allowed to form for 30 minutes followed by addition of Opti-MEM in 1:2 volume ratio and thorough mixing. The polyplexes were then matured 30 minutes prior to transfection or other assays such as DLS size measurement. Final concentration of pDNA in mature polyplex solution=3.3 μg/mL. Polyplexes made without PHQ doping may not be colloidally stable and aggregate to larger sizes (diameter>500 nm) within 30 minutes whereas PHQ inclusion helps particles avoid aggregation keeping the particle size smaller (under 200 nm) for up to 120 minutes (FIG. 4).

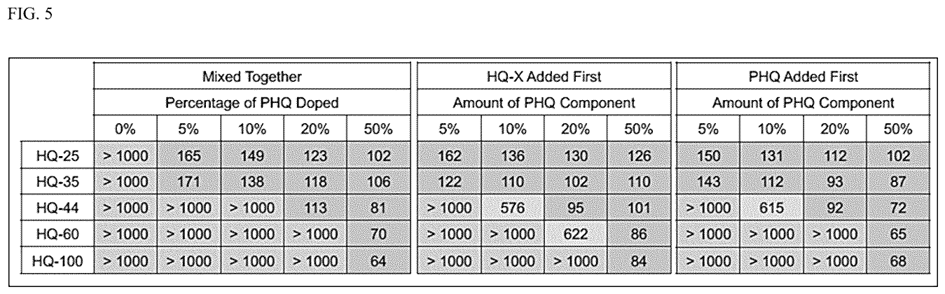

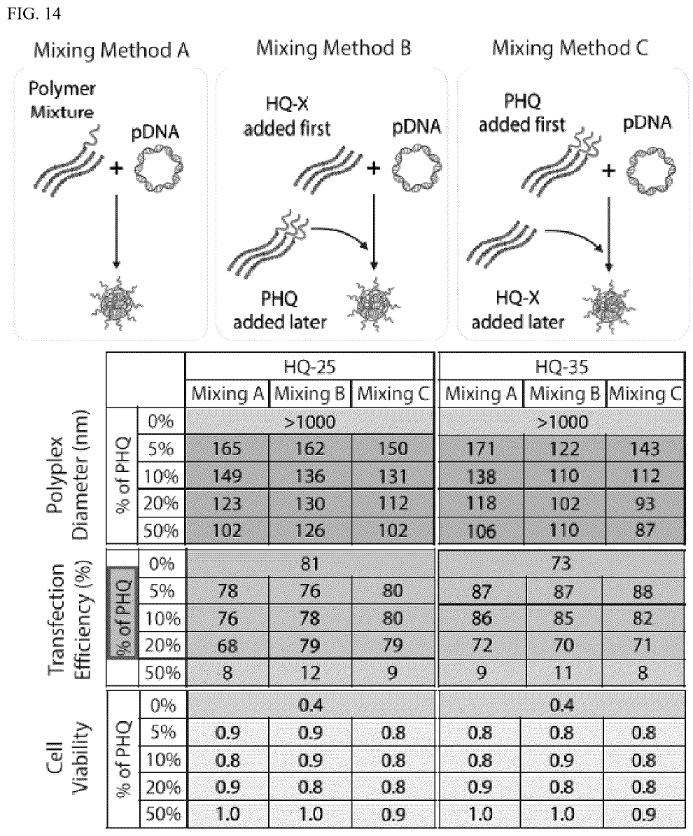

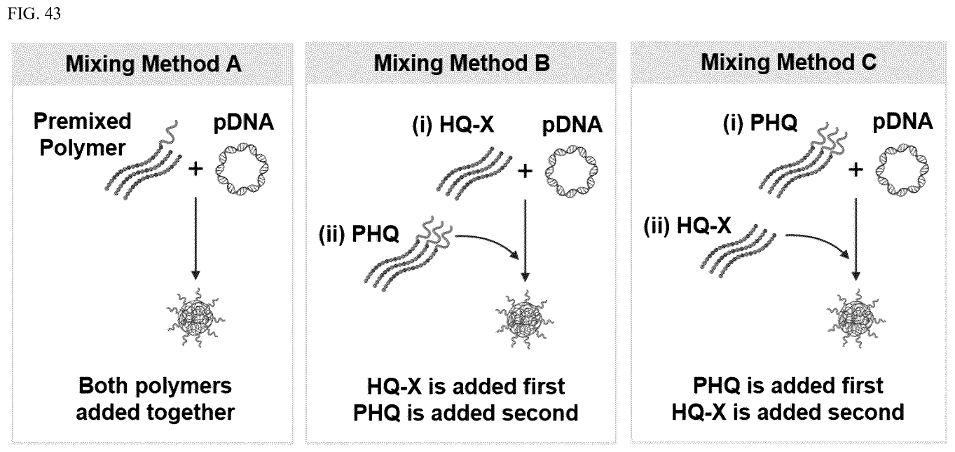

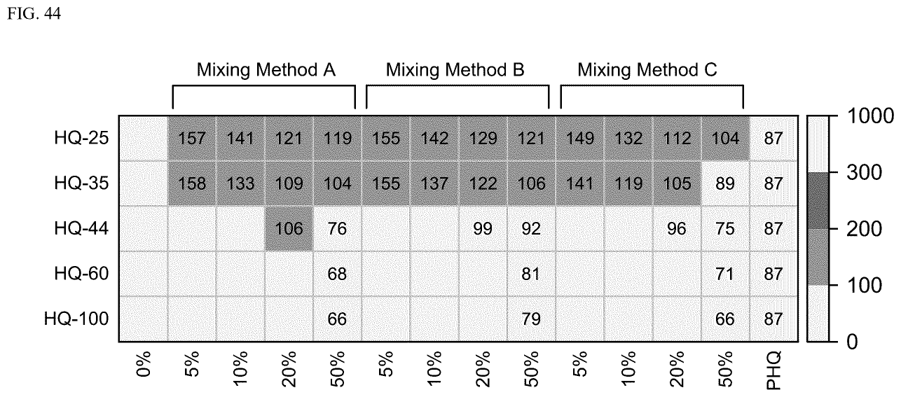

Following the procedure similar to the preparation of polyplexes described above, the effect of addition sequence of the hybrid polymer and the hydrophobic polymer into the transfection mixture was evaluated (FIG. 14 and FIG. 43). In Run-1, each of the five HQ-X polymers was individually mixed with PHQ before their addition to the pDNA solution followed by Opti-MEM addition. In Run-2, each of the five HQ-X polymers was added to the pDNA solution, followed by the addition of PHQ, followed by addition of Opti-MEM. In Run-3, PHQ was added to the pDNA solution first, followed by addition of each of the five HQ-X polymers, followed by Opti-MEM addition. FIGS. 5 and 44 show the sizes of the polyplexes formed from this experiment. Data presented in FIGS. 5 and 44 shows that all three sequences of addition lead to polyplex particles of the same size.

### Green Fluorescent Protein Reporter Assay

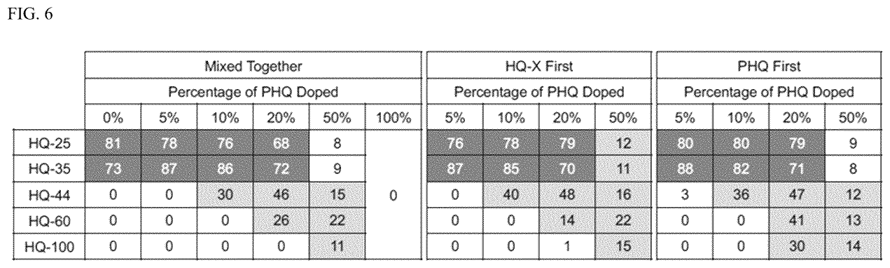

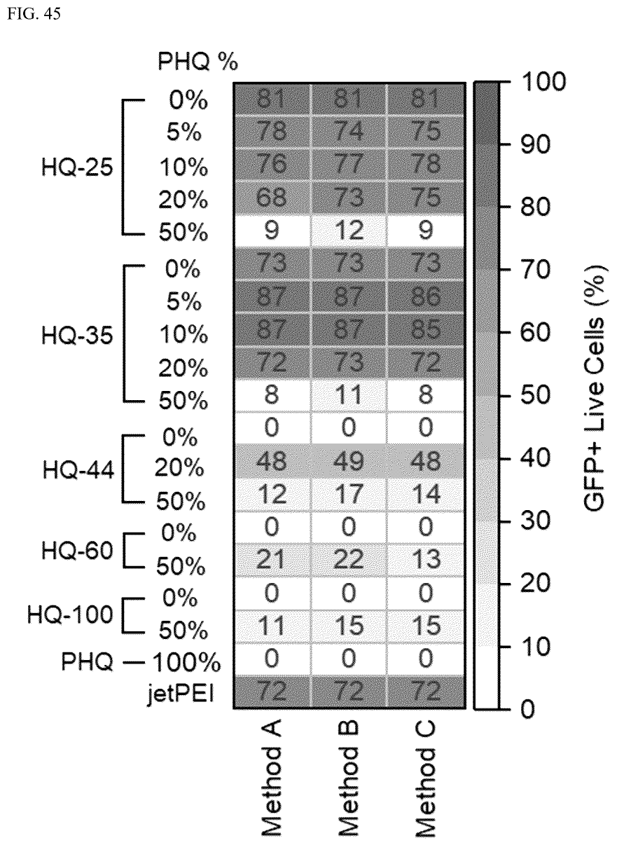

A green fluorescent protein reporter assay was performed using the pZsGreenl-N1 pDNA (4.7 kbp) as the payload to assess the ability of some polyplexes of the present disclosure to deliver pDNA into the nucleus. In this assay, HEK293T cells were transfected with the polyplex particles formed with polymer mixtures comprised of HQ-X and PHQ in different proportions (0% to 100%) (see FIG. 6 and FIG. 45) and pZsGreenl-N1. The transfection efficiency was evaluated based on the percentage of live cells having green fluorescence; that is, the percentage of live cells producing the green fluorescent protein (GFP), ZsGreen1.

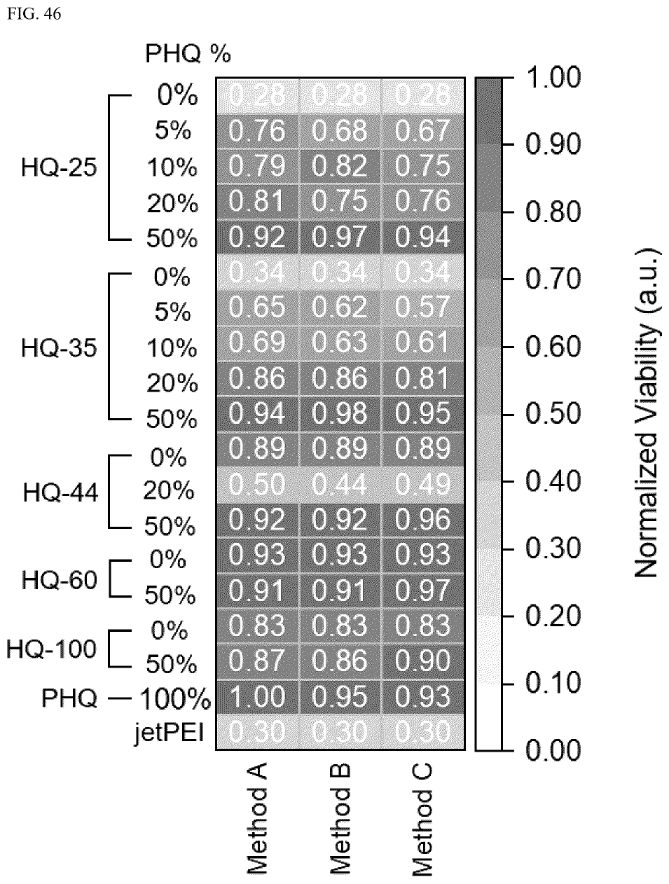

Polyplexes made using different mixing methods (see FIG. 43) were also tested. FIGS. 45, 46, and 47 show the GFP+ live cells (FIG. 45), normalized viability (FIG. 46), and effective efficiency of the polyplexes formed using different mixing methods testing in the green fluorescent protein reporter assay.

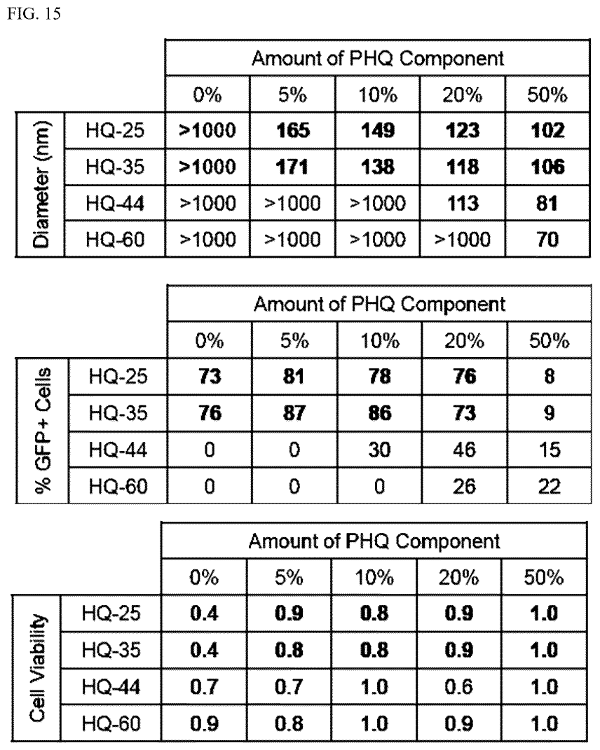

Additional polyplex size, GFP+ live cell, and cell viability data of polyplexes having various compositions is shown in FIG. 15.

### Quantifying Cell Viability

Cell viability was measured using colorimetric assay with CCK-8. 48 hours after transfection, the cells were treated with a 6% solution of CCK-8 in Phenol Red free DMEM and incubated at 37° C. with 5% CO2 for 1 hour. After 1 hour, the supernatant solution was transferred to a clear 96-well plate, and the absorbance of the supernatant solution at 450 nm was measured. Absorbance from 6% CCK-8 solution in FLUOROBRITE DMEM was subtracted from all data points and the values were normalized to the absorbance from the supernatant of untreated cells.

### Polyplex Internalization Studies

The extent of polyplex internalization was studied by transfecting cells using polyplexes that included the mixture of hybrid polymer (PHQ), the five HQ-X polymers, and Cy5-labeled pDNA.

Labeling pDNA with Cy5: LABEL-IT nucleic acid labeling kit from Mirus Bio (Madison, Wisconsin) was used to prepare Cy5-labeled pDNA using manufacturers protocol with one adjustment. Briefly, one full kit was used to label 1 milligram pDNA instead of 100 micro grams. The reduction in labeling density was to reduce alteration in polymer-pDNA binding while keeping sufficient number of fluorophores for detection using flow cytometry and confocal microscopy. Labeling density of the fluorescent probe was calculated using spectrophotometric method provided by Mirus Bio. The average ratio of nucleobase to Cy5 was calculated to be 440 which implies that each pDNA was labeled with 21 molecules of Cy5 on average.

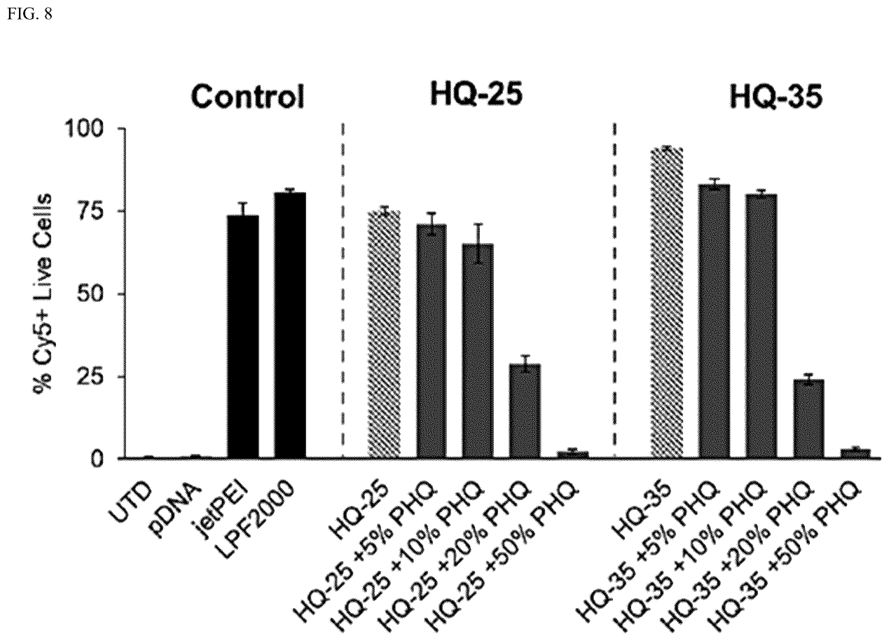

Polyplex internalization measurement using Cy5-Labeled pDNA: Cells were transfected according to the transfection protocol using the Cy5-labeled pZsGreen plasmid. 24 hours after transfection, the cells were washed with PBS and trypsinized using phenol red free trypsin for 5 minutes followed by quenching of trypsinization with serum supplemented phenol red free media. The cell suspension was diluted with ice cold PBS containing 2% fetal bovine serum and centrifuged at 4° C. at 1000 rpm for 10 minutes in deep well plates. The supernatant was removed, and the cells were incubated with a CELLSCRUB solution for 10 minutes at room temperature followed by centrifugation at 4° C. at 1000 rpm for 10 minutes. The supernatant was discarded, and the cells were resuspended in ice cold PBS with 2% fetal bovine serum and used for flow cytometry measurements. A 640 nm laser was used for detecting Cy5+ cells (FIG. 8).

### Dye Exclusion Assay

The ability of the mixture of a hybrid polymer (PHQ) and the five HQ-X polymers to bind with pDNA was evaluated using a dye exclusion assay.

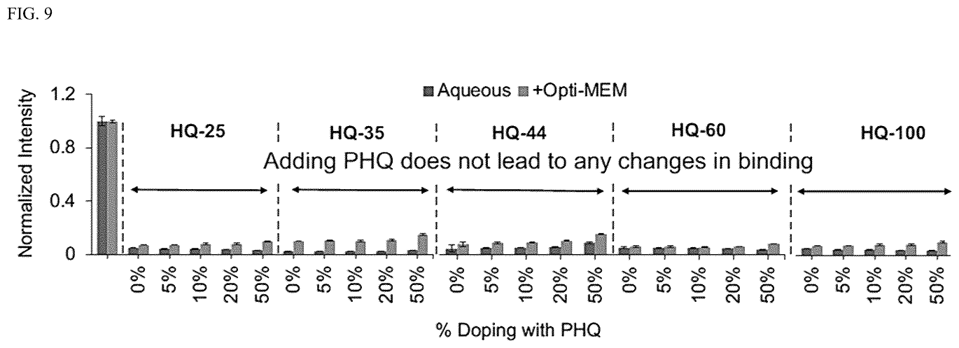

To improve the aqueous solubility of the polymers as well as to increase electrostatic interactions between the polymer chains and the pDNA, the stock solutions for the polymers were prepared in aqueous solution of acetic acid (3% glacial acetic acid in water, pH˜2.6). In general, for all experiments and assays, stock solutions of polymer (0.0104 M with respect of quinuclidine nitrogen atoms) and pDNA (1 mg/mL) were diluted with ultrapure water to freshly prepare secondary solutions with appropriate concentrations. pDNA was diluted to 0.02 mg/mL and the polymer solutions were diluted according to the desired N/P of final mixture. For the dye exclusion assay, pDNA was diluted with ultrapure water that was doped with PICOGREEN (0.5% v/v). The polymer secondary solution was added into the pDNA secondary solution in 1:1 volume ratio to form the primary polyplexes for 30 minutes at room temperature. Fluorescence intensity was measured using fluorescence filter cube (λₑₓ=485/20 nm, λₑₘ=528/20 nm). The intensity from the polyplex solutions (at respective N/P ratios) without PICOGREEN was used for background subtraction. The intensity from the polymer free solutions (N/P=0) were used to normalize the intensity from the polyplex solutions. Additionally, fluorescence of the polymers in the presence of PICOGREEN but without pDNA was measured for which only baseline level fluorescence signal was observed. These controls confirmed that neither the fluorescence of the free polymer nor the interaction of PICOGREEN with polymer interferes with the dye exclusion results (FIG. 9).

### Synthesis of PEG-CTA

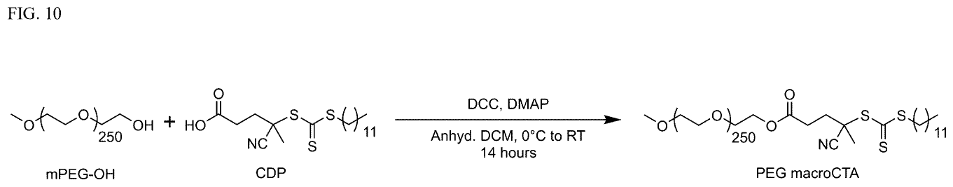

FIG. 10. shows the synthetic scheme used to synthesis PEG-CTA. Into an oven dried round bottom flask, mPEG-OH (5 g, 0.48 mmol, 1 equiv.), CDP (394 mg, 0.96 mmol, 2 equiv.), and DMAP (59.2 mg, 0.48 mmol, 1 equiv.) were transferred and dissolved in 25 mL of anhydrous DCM and stirred well until all solids were dissolved. The reaction mixture was then immersed in an ice bath to cool down. Into this ice cold solution, DCC (198 mg, 0.96 mmol, 2 equiv.), dissolved in 2 mL anhydrous DCM, was added drop wise under constant stirring. After 2 hours, the reaction mixture was placed at room temperature and stirred for another 22 hours. The product was initially recovered as a yellow waxy substance by precipitation in 400 mL of cold diethyl ether followed by vacuum filtration. The filtered product was then redissolved in 30 mL of DCM followed by reprecipitation in 400 mL of cold diethyl ether overnight in freezer. This process was repeated two more times before vacuum filtration. The filtered product was then dried overnight in vacuum oven at 50° C. to obtain the final product in 4.1 g (76%) yield.

### Example 1 Data and Analysis

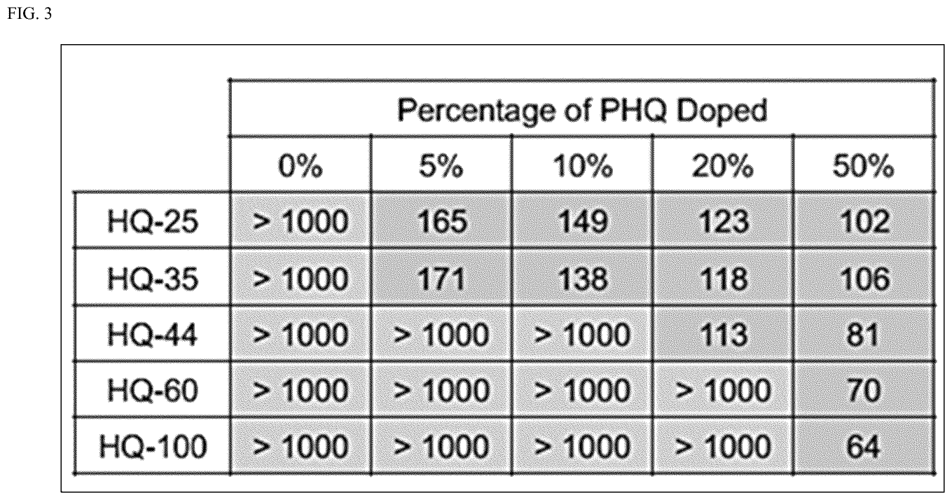

FIG. 3 is a table summarizing polyplex particle diameter sizes (in nanometers) of different polyplex compositions formed with pDNA and different PHQ and HQ-X mixtures. In the absence of PHQ, all five HQ-X polymers showed aggregation of polyplex particles with particle diameters greater than 1000 nanometers. All five HQ-X polymers showed polyplex particles with diameters less than 200 nanometers with 50% PHQ doping (FIG. 3).

The ability of the mixtures of hybrid polymers to help deliver pDNA was studied using a green fluorescent protein reporter assay. HQ-25 and HQ-35 with PHQ performed efficiently displaying greater than 60% efficiency in the assay (FIG. 6). PHQ-only samples were unable to deliver pDNA in the assay.

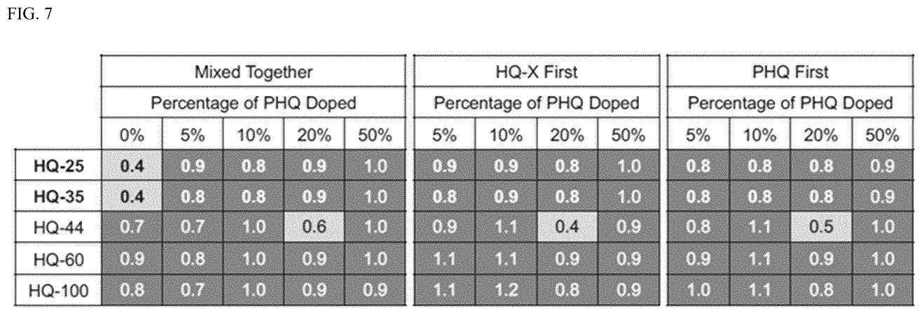

Cell viability of the transfected cells was measured using colorimetric assay with CCK-8. Data shown in FIGS. 7 and 46 depicts values normalized from 0 to 1. A value of 1 means 100% survival of the cells. The results showed that for the HQ-25 and HQ-35 polymers, the cell survival improved with PHQ doping starting at 5% PHQ doping level. These data showed that adding PHQ helped avoid particle aggregation, helped achieve better performance in delivering pDNA, and improved cell viability.

The effect of the addition sequence of the hybrid polymer and the hydrophobic polymer into the transfection mixture was also evaluated. Each of the five HQ-X polymers was individually mixed with PHQ before their addition to the transfection mixture. The results were compared to a run where the HQ-X polymer was added to the transfection mixture, followed by the addition of PHQ, and a run in which PHQ was added to the transfection mixture first, followed by the addition of the HQ-X polymer. Data presented in FIG. 5 shows that all three sequences of addition lead to polyplex particles of the same size.

The ability of the mixture of a hybrid polymer (PHQ) and the five HQ-X polymers to bind with pDNA was evaluated using a dye exclusion assay. FIG. 9 shows the results. Adding PHQ to a polyplex formulation of HQ-25, HQ-35, HQ-44, HQ-60, and HQ-100 did not lead to any changes in binding compared to the HQ-X alone.

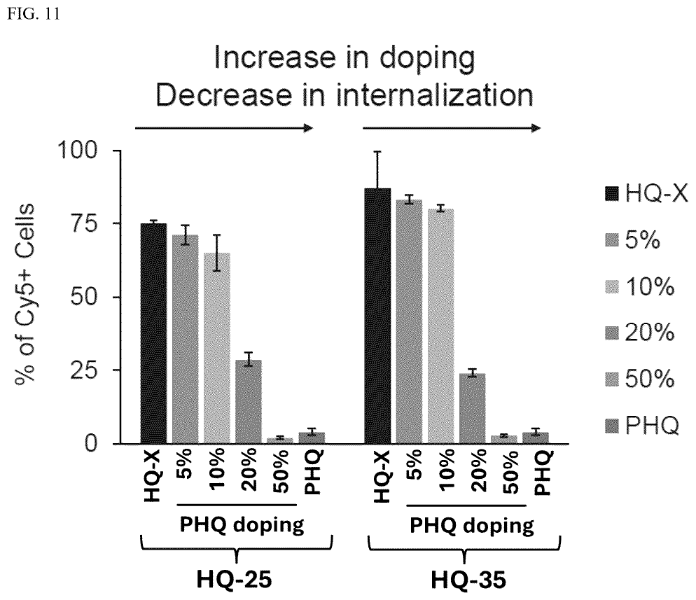

The extent of polyplex internalization was studied by transfecting cells using polyplexes that included the mixture of hybrid polymer (PHQ), the five HQ-X polymers, and Cy5-labeled pDNA. FIGS. 8 and 11 show that increasing the amount of PHQ leads to a decrease in cellular uptake of the polyplexes evident by the decrease in the percentage of Cy5+ cells.

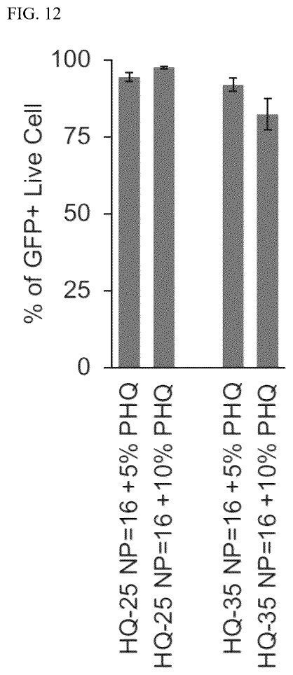

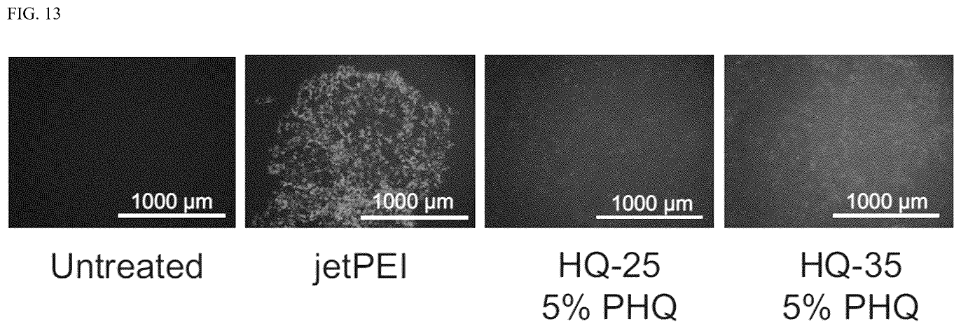

A green fluorescent protein reporter assay was performed using the pZsGreenl-N1 pDNA (4.7 kbp) as the payload to assess the ability of some polyplexes of the present disclosure to deliver pDNA into the nucleus. FIGS. 12 and 13 shows the results.

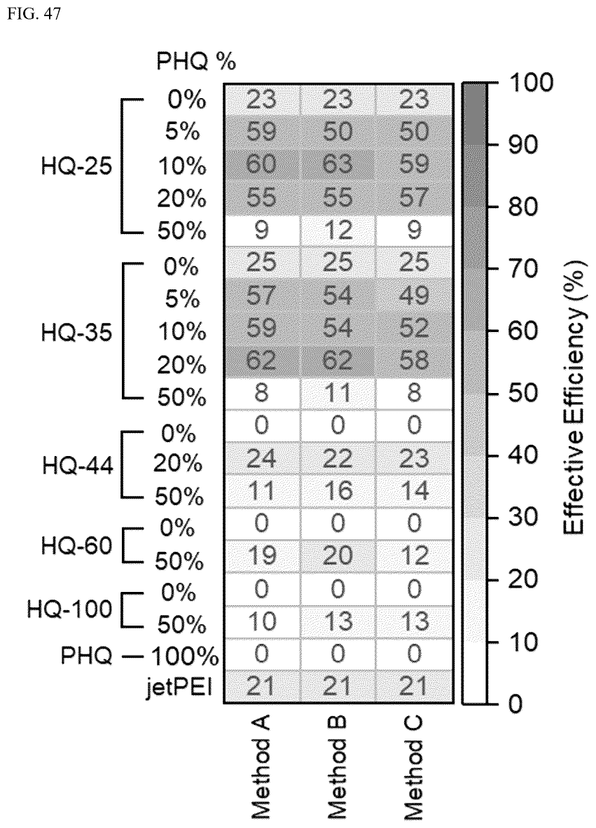

Three different mixing sequences were performed to understand if the size of the final polyplexes is influenced by the order of polymer addition to pDNA. FIG. 43 shows an overview of the three different mixing methods tested. FIG. 44 shows diameter of 70 total polyplex formulations tested. In the absence of PHQ, all five HQ-X polymers lead to colloidally unstable polyplexes. As PHQ % increases, colloidal stability is achieved. For HQ-25, and HQ-35, even 5% PHQ is enough to impart colloidal stability. For HQ-44, HQ-60, and HQ-100 which are more hydrophobic, higher PHQ % was needed to achieve colloidal stability. In general, with the increase in PHQ %, the average polyplex size also decreased. When comparing polyplex formulations across the three mixing sequences, no significant differences were observed suggesting all three mixing methods lead to similar polyplexes. The transfection ability of the colloidally stable polyplexes formed using the three different mixing sequences was evaluated. FIG. 45, FIG. 46, and FIG. 47 show the transfection efficiency of polymer mixtures in terms of percentage of GFP+ live cells, the normalized viability of the cells after transfection using polymer mixtures, and the effective efficiency of transfection, respectively. Effective efficiency is the product of transfection efficiency and normalized cell viability, and it illustrates a whole view of performance by the pDNA carriers. For comparison, polyplexes formed with only the HQ-X component as well as only PHQ were also evaluated. While pure HQ-X polyplexes showed high transfection efficiency and low viability, the polymer mixtures generally showed retention of transfection efficiency and improvement in cell viability. Pure PHQ had no GFP+ cells. Along those lines, polymer formulations with 50% PHQ incorporation also had a low transfection efficiency. All transfections were accompanied by cells transfected with jetPEI, a commercially available transfection agent.

### Example 2

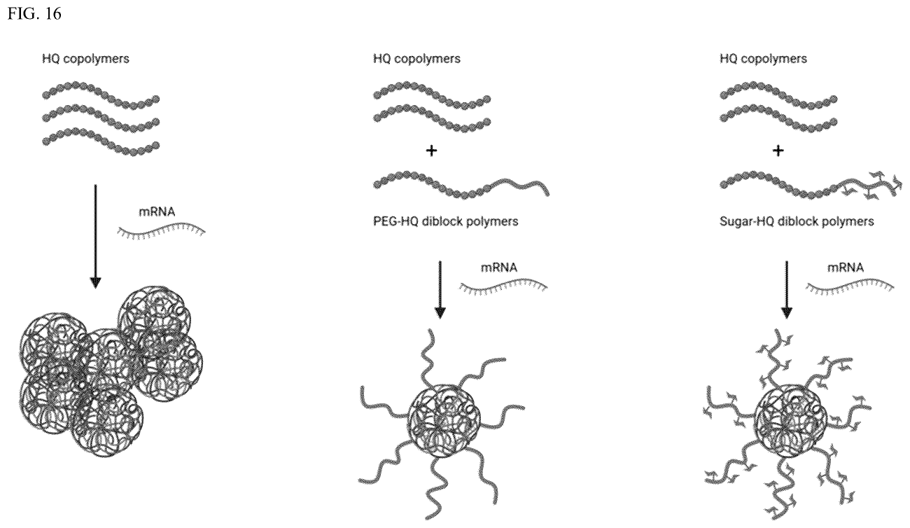

Example 2 explores the properties and characteristics of polyplexes that include mRNA, a hybrid polymer, and a hydrophobic polymer. Three different hybrid polymers were tested. The first hybrid polymer included a hydrophobic block polymerized from 2-hydroxyethyl acrylate (HEA) and a hydroquinine monomer (HQ) as well as a hydrophilic block polymerized from a glucose-containing monomer (abbreviated as GlcHQ polymer or simply GlcHQ; FIG. 16). The first hybrid polymer included a hydrophobic block polymerized from 2-hydroxyethyl acrylate (HEA) and a hydroquinine monomer (HQ) as well as a hydrophilic block polymerized from a galactose-containing monomer (abbreviated as GalHQ polymer or simply GalHQ; FIG. 16). The third hybrid polymer included a hydrophobic block polymerized from 2-hydroxyethyl acrylate (HEA) and a hydroquinine monomer (HQ) as well as a polyethylene glycol hydrophilic block (abbreviated as PHQ polymer or simply PHQ; FIG. 16). The hydrophobic polymer was polymerized from 2-hydroxyethyl acrylate (HEA) and the same hydroquinine monomer (HQ) as used in the hybrid polymers (abbreviated as HQ polymer or simply HQ).

For polyplex formation, 72 polymer formulations were tested, each formulation including one hybrid polymer and one hydrophobic polymer. In the formulation series, the GalHQ, GlcHQ, or PHQ hybrid polymers were kept constant while HQ polymers varied by hydroquinine content. More specifically, HQ polymers were synthesized that had 25 mol-% (HQ-25), 35 mol-% (HQ-35), 44 mol-% (HQ-44), 60 mol-% (HQ-60), and 100 mol-% (HQ-100) hydroquinine content.

Binary mixtures of GalHQ, GlcHQ, or PHQ and HQ-X (where X is the mol-% of HQ in the polymer) were formulated based on the contribution of each polymer to the overall N/P ratio, or the molar ratio of quinuclidine nitrogen atoms to the phosphate groups in the mRNA. For example, an N/P of 16 indicates there are 16 times more quinuclidine moieties than phosphate groups in the formulation. A formulation having 10% GalHQ, GlcHQ, or PHQ and 90% HQ-X indicates that the contribution from HQ-X in this mixture is 14.4 and the contribution from PHQ, GlcHQ, or GalHQ is 1.6. Each of HQ-25, HQ-35, HQ-44, HQ-60, and HQ-100 where separately formulated with 5%, 10%, 20%, 50%, or 75% PHQ; 5%, 10%, 20%, 50%, or 75% GlcHQ; or 5%, 10%, 20%, 50%, or 75% GalHQ. Additionally, formulations of 100% PHQ, GlcHQ, and GalHQ were tested.

### Materials

Chemical Materials: Hydroquinine (98%), 2-hydroxyethylacrylate (HEA), Acryloyl chloride, 2-(Dodecylthiocarbonothioylthio)-2-methylpropionic acid (DDMAT), 4-cyano-4 [(dodecylsulfanylthiocarbonyl)sulfanyl]pentanoic acid (CDP), 2,2′-Azobis(2-methylpropionitrile) (AIBN), 4,4′-azobis(cyanovaleric acid) (V-501), N,N′-dicyclohexylcarbodiimide (DCC), silica gel (technical grade, pore size 60 Å, 70-230 mesh), anhydrous N,N-dimethylformamide (DMF) and dichloromethane (DCM), were purchased from Sigma-Aldrich (Saint Louis, MO). 2-Isocyanoethyl acrylate (stabilized with BHT) was purchased from Tokyo Chemicals Inc. Americas (Portland, OR). D-glucosamine hydrochloride and 4-dimethylaminopyridine (DMAP) were purchased from Acros Organics (Geel, Belgium). D-galactosamine hydrochloride was purchased from Carbosynth (Staad, Switzerland). mPEG-OH, 10 kDa, was purchased from Creative PEGWorks (Chapel Hill, NC). Spectra/Por dialysis tubing with a molecular-weight cutoff of 1 kDa, treated with 0.1 wt % ethylenediaminetetraacetic acid (EDTA) solution and stored in ˜0.05 wt % sodium azide, was purchased from Spectrum Chemical (New Brunswick, NJ).

Biological Materials: Dubelco's Modified Eagle Medium (DMEM; high glucose, pyruvate and Glutamax supplemented), Ham's F-12K (Kaighn's) Medium, Roswell Park Memorial Institute (RPMI) 1640 Medium, Opti-MEM Reduced Serum Medium, Heat Inactivated Fetal Bovine Serum (HI FBS), Antibicrobial/Antimycotic, Phosphate Buffered Saline (PBS, pH=7.4), Fluorobrite DMEM, Trypsin-EDTA (0.5%), CellTrace Calcein Violet, Quant-iT RiboGreen RNA assay kit, and UltraPure DNase/RNase-free water were all purchased from Life Technologies-ThermoFisher Scientific (Carlsbad, CA). PAGE GelRed Nucleic Acid Gel Stain was purchased from Biotium (San Francisco, CA). CCK-8 cell counting kit was purchased from Dojindo Molecular Technologies (Rockville, MD). DasherGFP mRNA was purchased from Aldeveron (Fargo, ND). EZ Cap Cy5 EGFP mRNA (5-moUTP) and ARCA Cy3 EGFP mRNA (5-moUTP) were purchased from APExBIO (Houston, TX). jetPEI was purchased from PolyPlus (Illkirch, France). Lipofectamine 2000 (LPF 2000) was purchased from Invitrogen (Waltham, MA). Murine CD1 red blood cells were purchased from Innovative Research Inc. (Novi, MI). The HEK-293T cell line was engineered to have the traffic light reporter system in the laboratory of Mark Osborne at the University of Minnesota and the subclones were generated at the Genome Engineering Shared Resource (Minneapolis, MN) to ensure a stable cell line. The A549 and MDA-MB-231 cell lines were purchased from ATCC (Manassas, VA). The HuH-7 cell line was purchased from Cytion Cell Lines Services (Sioux Falls, SD).

### Monomer Synthesis

Monomer Synthesis. HQ monomer was previously synthesized and characterized according to Roy, P.; Kreofsky, N. W.; Brown, M. E.; Van Bruggen, C.; Reineke, T. M. Enhancing pDNA Delivery with Hydroquinine Polymers by Modulating Structure and Composition. JACS Au 2023, 3 (7), 1876-1889. Sugar monomers, 2-deoxy-2-acrylamido glucopyranose (AAGlc) and 2-deoxy-2-acrylamido galactopyranose (AAGal), were synthesized following a previously reported method with slight modification (Lan, T.; Guo, H.; Lu, X.; Geng, K.; Wu, L.; Luo, Y.; Zhu, J.; Shen, X.; Guo, Q.; Wu, S. Dual-Responsive Curcumin-Loaded Nanoparticles for the Treatment of Cisplatin-Induced Acute Kidney Injury. Biomacromolecules 2022, 23 (12), 5253-5266). D-glucosamine hydrochloride or D-galactosamine hydrochloride (5.1 g, 23.5 mmol), potassium carbonate (3.2 g, 23.5 mmol), and methanol (125 mL) were added to a round bottomed flask and cooled to 0° C. using an ice bath. Acryloyl chloride (2.3 g, 25 mmol) was added to the flask using a syringe pump over 30 min. The reaction stirred for 30 minutes in the ice bath then allowed to stir for 3 hours at room temperature. 100 mL of cold methanol was added to precipitate out the salts and the mixture was filtered. The filtrate was then concentrated using rotary evaporation and dry loaded onto a silica gel column. The mobile phase used for the purification of the product was 4:1 ethyl acetate:methanol. Yields for AAGlc and AAGal were 39% and 43%, respectively.

### Polymer Synthesis

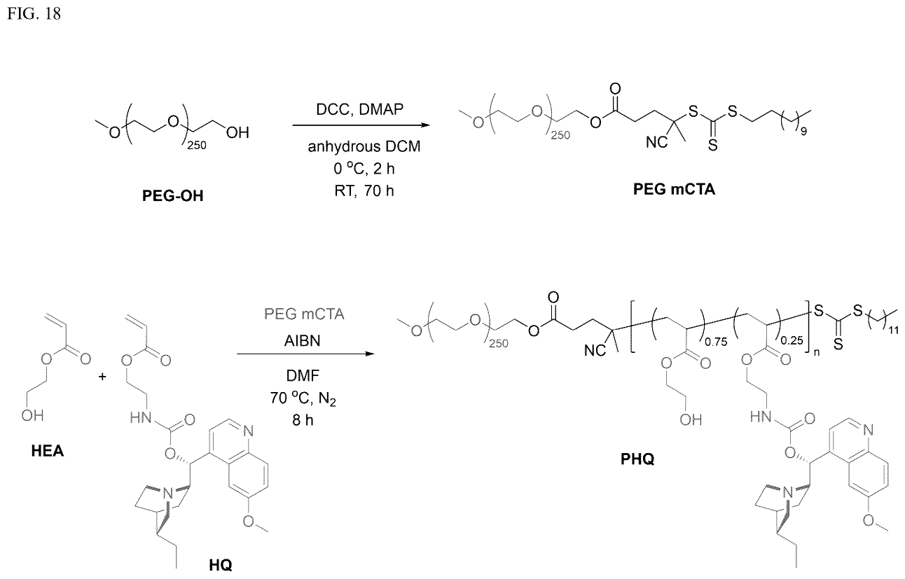

HQ-X polymers in the binary mixtures were previously synthesized and characterized according to Roy, P.; Kreofsky, N. W.; Brown, M. E.; Van Bruggen, C.; Reineke, T. M. Enhancing pDNA Delivery with Hydroquinine Polymers by Modulating Structure and Composition. JACS Au 2023, 3 (7), 1876-1889. PEG macroCTA was synthesized as previously reported, with slight modification (see FIG. 18; Roy, P.; Kreofsky, N. W.; Santa Chalarca, C. F.; Reineke, T. M. Binary Copolymer Blending Enhances pDNA Delivery Performance and Colloidal Shelf Stability of Quinine-Based Polyplexes. Bioconjugate Chem. 2025.) Briefly, PEG-OH (1 g, 100 umol), CDP (80.5 mg, 200 umol), and DMAP (12 mg, 100 umol) were transferred and dissolved in anhydrous DCM (10 mL) then cooled to 0° C. in an ice bath. DCC (41 mg, 200 umol), dissolved in DCM, was added dropwise. After 2 hours, the reaction mixture was allowed to warm to room temperature and stirred for another 70 hours. The product was precipitated using cold diethyl ether and dried in under high vacuum overnight (yield=95%). All polymerizations were done using RAFT (see FIG. 18). Any monomers with inhibitor present were passed through an activated basic alumina plug prior to the reaction. Generally, all reactants were dissolved in a vial and the reaction mixture was sparged with nitrogen gas for 30-60 minutes depending on reaction volume before stirring at 70° C. for the duration of the reaction. Reactions were quenched by freezing with liquid nitrogen and exposing them to atmospheric oxygen. HQ-25 was resynthesized to serve as part of the sugar diblock polymers: HQ (857 mg, 1.83 mmol), HEA (561 mg, 4.83 mmol), DDMAT (12 mg, 0.033 mmol), and AIBN (0.55 mg, 0.0033 mmol) were dissolved in DMF (5 mL) and polymerized via RAFT for 6.5 hours. Chain extension of HQ-25 with AAGlc and AAGal was accomplished in much the same way: AAGlc or AAGal (256 mg, 1.1 mmol), HQ-25 macro-CTA (253 mg, 0.011 mmol), and AIBN (0.18 mg, 0.0011 mmol) were dissolved in DMF (2.2 mL) and polymerized via RAFT for 6 hours. PHQ was synthesized with RAFT polymerization as well in a similar manner: HQ (514 mg, 1.1 mmol), HEA (336 mg, 2.9 mmol), PEG macro-CTA (220 mg, 0.02 mmol), and V501 (0.56 mg, 0.002 mmol) were dissolved in DMF (5 mL) and polymerized via RAFT for 8 hours. HQ-25 and PHQ were both purified using 1 kDa MWCO dialysis tubing and 75/25 methanol/tetrahydrofuran as the solvent. These pure polymers were dried in a vacuum oven at 40° C. overnight to obtain pale yellow powders. GlcHQ and GalHQ were both purified via dialysis in 1 kDa MWCO dialysis bags using water that had been adjusted to pH 4.5 by dropping in 1M HCl. The pure polymers were lyophilized resulting in a fluffy white powder as the final product. Reaction conditions are shown in table 1.

TABLE 1

| | % | | | | | |
|---|---|---|---|---|---|---|
| | HQ in | | | | | Scale |
| | mono- | | | [Mono- | % | (mmol |
| | mer | CTA:Mono- | CTA:Ini- | mer] | Con- | mono- |
| Name | feed | mer | tiator | (M) | version | mer) |
| HQ-25 | 27.5 | 200:1 | 0.1:1 | 1 | 72 | 6.67 |
| GlcHQ | — | 100:1 | 0.1:1 | 0.5 | 62 | 1 |
| GalHQ | — | 100:1 | 0.1:1 | 0.5 | 63 | 1 |
| PHQ | 27.5 | 200:1 | 0.1:1 | 0.8 | 60 | 4 |

### Chemical Characterization

All pure products were characterized via ¹H NMR spectroscopy on a Bruker (Billerica, MA) Advance III HD 500 MHz NMR spectrometer using chloroform-d or DMSO-d6 as the solvent. For each measurement 32 scans were collected with a relaxation delay of 10 seconds. For the sugar diblock polymers, GlcHQ and GalHQ, ¹H NMR spectroscopy was the primary means of determining molar mass because the diblock polymers were incompatible with the columns installed on the size exclusion chromatography (SEC) system using a DMF mobile phase and the polymers aggregated/micellized slightly in the aqueous mobile phase used on a different SEC system with compatible columns. As a result, the molar masses and dispersities are likely overestimated. For other polymers, HQ-X and PHQ, molar mass was determined using an SEC system using DMF with 0.5 M LiBr as the mobile phase, equipped with an Agilent (Santa Clara, CA) Infinity 1200 HPLC system, a Wyatt (Santa Barbara, CA) miniDAWN multiangle laser light scattering detector, and a Wyatt OPTILAB T-rEX refractive index detector. The dn dc of several polymer building blocks was measured (HQ-25, pAAGlc, pAAGal) on a Wyatt OPTILAB T-rEX refractive index detector so that the weight averaged approximation could be used to determine the dn dc of the polymers studied:

$$\frac{\partial n}{\partial c} = {{w}_{A} ( \frac{\partial n}{\partial c} )}_{A} + {{w}_{B} ( \frac{\partial n}{\partial c} )}_{B}$$

where W_{A} is the weight fraction of block A, W_{B} is the weight fraction of block B, (dn/dc)_{A} is the dn/dc of block A, and (dn/dc)_{B} is the dn/dc of block B.

### Polyplex Formation

Binary mixtures were formulated based on the contribution to the overall N/P ratio, or the molar ratio of quinuclidine nitrogen atoms to the phosphate groups in the mRNA. For example, an N/P of 16 indicates there are 16 times more quinuclidine moieties than phosphate groups in the formulation. A 10% mixture of HQ-X and diblock stabilizer indicates that the contribution from HQ-X in this mixture is 14.4 and the contribution from the diblock stabilizer (PHQ, GlcHQ, GalHQ) is 1.6. Polymer stock solutions were prepared at 10 nmol N/μL in 3% acetic acid (pH˜2.6) then combined to generate all the percent stabilizer formulations (5%, 10%, 20%, 50%, 75%, and 100%). These mixtures were then diluted to give the appropriate concentration for N/P=16 and mixed with mRNA (0.02 μg/μL) in a 1:1 ratio. For all biophysical studies, unless otherwise noted, the mRNA used was Aldeveron DasherGFP mRNA, while for transfection studies the mRNA used was APExBIO Cy5 Labeled EGFP mRNA. The solution was given 30 minutes to complex before the addition of buffer (Opti-MEM or in some cases, PBS+50 mM NaHCO₃) in a 2:1 ratio to give a final mRNA concentration of 3.33 g/mL. The addition of buffer brought the pH of the solution to ˜6.7 in Opti-MEM and ˜7.4 in the PBS+50 mM NaHCO₃.

### Dynamic Light Scattering

Polyplex sizes and PDI values were measured on a Wyatt DynaPro Plate Reader III at the same concentration that was used for transfection (3.3 g/mL mRNA) in various buffers (Opti-MEM and PBS+50 mM NaHCO₃) (See “Polyplex Formation”). For each mixture, 5-7 acquisitions were acquired with an acquisition time of 5 seconds at 25° C. For the long-term stability studies, the polyplex solutions were stored in the fridge at 4° C. between measurements. Prior to each measurement the solutions were thoroughly mixed using a pipette. Instrument parameters remained the same for all these DLS measurements.

### Dye Exclusion Assay

Polyplexes were formed as described in the “Polyplex formation” at three N/P ratios (4, 8, and 16) stopping after the complexation stage before adding Opti-MEM. Instead of diluting the mRNA to 0.2 μg/μL with water, the mRNA stock solution was diluted with water containing RiboGreen dye (0.5% v/v). The fluorescence of RiboGreen was measured on a BioTek (Winooski, VT) Synergy H1 Hybrid Reader using a filter cube (λₑₓ=485/20 nm, λₑₘ=528/20 nm). Controls of polyplexes without dye were used to background subtract and the signal was normalized to an N/P=0 (no polymer) control. After measuring the polyplexes in acidic water, Opti-MEM was added in the standard 2:1 ratio and the fluorescence of the samples was remeasured after 30 minutes.

### Gel Migration Assay

Agarose gel (1.5% w/v) made with Tris-EDTA and spiked with 1×GelRed Nucleic Acid Gel Stain was poured and allowed to solidify. Polyplexes were prepared as described in the “Polyplex formation” section at N/P=16. To get adequate signal from the mRNA in the gel, the polyplexes were concentrated to be ˜10× higher concentration than the standard transfection concentration using Amicon Ultra Centrifugal Filters (30 kDa MWCO) purchased from Sigma-Aldrich. After thirty minutes of polyplex annealing and an additional thirty minutes of incubation with buffer, the polyplex solution was added to the filter unit, spun at 14,000 g for 5 minutes. The filter was inverted and the tubes were spun again at 1,000 g for 2 minutes to collect the concentrated solution. 20 μL of this concentrated solution was added to 10 μL of 3× gel loading buffer and transferred to the wells of the gel. The assay was run at 70 V for one hour in Tris-EDTA buffer. Images were captured using a UV gel transilluminator and a Google (Mountain View, CA) Pixel 6 Pro camera.

### FRET Assay

Polyplex solutions were formed in the same way as described in the “Polyplex formation” section at N/P=16, with a couple of exceptions. The buffer used to dilute the samples was Fluorobrite DMEM because of its minimal disruption to fluorescence. The mRNA used to form these polyplexes were Cy3 labeled EFGP mRNA and/or Cy5 labeled EGFP mRNA from APExBIO. Three conditions were tested for each formulation: A negative FRET control of polyplexes formulated with just Cy3 mRNA, a positive FRET control of polyplexes co-formulated with half Cy3 mRNA and half Cy5 mRNA, and a test condition of polyplexes made separately with Cy3 mRNA and Cy5 mRNA, then mixed together prior to Fluorobrite DMEM addition. Once the Flourobrite DMEM was added, 30 minutes of incubation time was given to the formulations to lock in the particles, then each of these polyplex solutions were concentrated down in the same way as described in the “Gel Electrophoresis” section to give ˜10× concentration for better fluorescence signal. Samples were diluted in Fluorobrite DMEM to give a consistent concentration of Cy3 mRNA in each sample then the emission spectra were measured on a BioTek Synergy H1 Hybrid Reader with λₓ=500 nm. For the PHQ containing samples, triplicate data points were collected, while for the GlcHQ and GalHQ containing samples, data was collected in duplicate. Cy3 emission at 570 nm was used to compare the three conditions tested. To determine FRET efficiency, the following equation was used where I_{DA} is the intensity of donor (Cy3) in the presence of acceptor (mixed condition) and I_{D} is the intensity of donor (Cy3) without acceptor (negative control):

$${E}_{FRET} = ( 1 - \frac{{I}_{DA}}{{I}_{D}} ) * 100 \%$$

### Cell Culturing

The IEK-293T and MDA-MB-231 cell lines were cultured in supplemented high glucose DMEM (with Glutamax and pyruvate). The A549 cell line was cultured in supplemented Ham's F-12K (Kaighn's) Medium (with L-Glutamine). The HuH-7 cell line was cultured in supplemented RPMI 1640 Medium (with L-Glutamine). All media were supplemented with 10% FBS and 1% Antibiotic/Antimycotic. All cell lines were grown in 75 cm² cell culture flasks for adherent cell types and incubated in a humidified incubator at 37° C. and 5% CO₂. Cells were regularly passaged to maintain less than 80% confluency. Transfections were done when the cell density was 70-80% confluency.

### Transfection/Internalization Reporter Assay

A transfection reporter assay was used to determine the transfection efficiency, relative cell viability, and internalization for the polyplexes presented in this work. The EZ Cap Cy5 EGFP mRNA (5-moUTP) from APExBIO was used as the cargo for all transfection studies. For all cell lines presented in this work the same transfection procedure was followed because they are all adherent cell types. 24 hours prior to transfection, the cells were removed from their culture flasks and plated at a density of 50,000 cells/mL in 48-well plates (500 μL per well). After giving the cells 24 hours to proliferate, polyplexes were formed in the same way as described in the “Polyplex Formation” section at a final concentration of 3.33 μg/mL in serum-free Opti-MEM. 150 μL of this polyplex solution was transferred to each well after aspirating off the plating media and rinsing with PBS (0.5 g mRNA/well). The cells were placed back into the incubator for 2 hours, then the transfection was quenched with serum-supplemented growth media. Both jetPEI (N/P=5) and LPF 2000 were used per manufacturer guidelines as positive controls. 24 hours after transfection, the cells were prepared for analysis. To do this, the cells were trypsinized to remove them from the 48-well plates and aliquoted into two plates—one for flow cytometry and the other for a cell counting kit-8 (CCK-8) viability assay. For the CCK-8 assay, cells were given CCK-8-spiked supplemented DMEM (8% v/v) to measure the metabolic activity of the cells. After 1-3 hours of incubation (depending on the metabolic rate of the cell line) absorbance at 450 nm was measured on a BioTek Synergy H1 Hybrid Reader. For the flow cytometry aliquot of cells, they were centrifuged down, then resuspended in PBS. This was done a total of 3 times to remove polyplexes that were outside of the cells to not interfere with the internalization results. After the last rinse with PBS, the cells were resuspended in 2% FBS containing PBS spiked with CellTrace Calcein Violet as a viability stain. After allowing 15 minutes for dye penetration, the cells were analyzed on a Bio-Rad (Hercules, CA) ZE5 flow cytometer with 488 nm, 640 nm, and 405 nm laser channels activated. This allowed for determination of Calcein Violet, EGFP, and Cy5 fluorescence.

### Statistical Analysis

All statistical analyses were completed in GraphPad Prism v9.5.0. For transfection data, statistical analysis was performed using one-way ANOVA followed by a post-hoc Tukey test to determine the statistical significance of pairs within each formulation. For FRET studies, statistical analysis was done using an unpaired t-test to compare to the negative Cy3 only control.

### Example 2 Data and Analysis

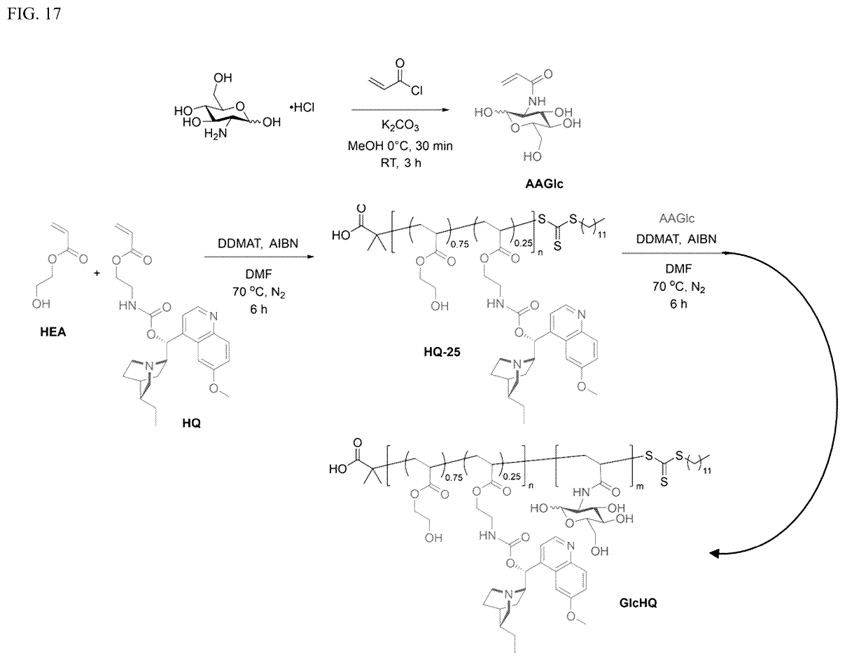

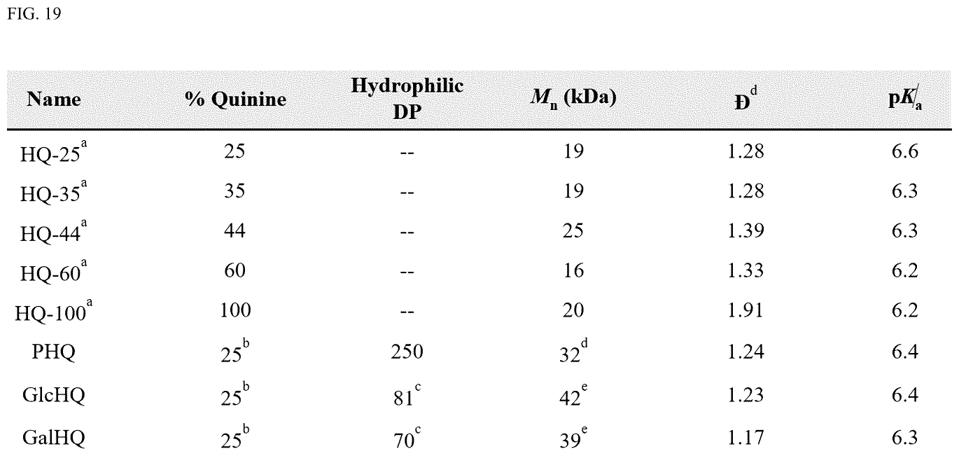

To achieve controlled, living polymerization, an acrylate-functionalized quinine derivative (HQ) was synthesized. This monomer was amenable to the generation of a library of statistical copolymers containing 2-hyrdoxyethyl acrylate (HEA) and HQ using reversible addition fragmentation chain transfer (RAFT) polymerization. This collection of polymers is referred to as HQ-X (FIG. 17) where X is the molar percentage of HQ in the polymer, ranging from 25-100% (i.e. HQ-25 contains 25 mol % HQ and 75 mol % HEA). A previous batch of these polymers that had been thoroughly characterized was used for the studies contained in this work (FIG. 19). To generate the PEGylated HQ-25 diblock polymer (PHQ) we utilized our previously reported method.⁴³ PEG-OH was coupled to a CDP chain transfer agent (CTA) using DCC coupling (FIG. 17) to generate a macroCTA. This macroCTA was then chain extended with HQ and HEA in a 25/75 feed ratio using RAFT polymerization to generate a diblock polymer with a 10 kDa PEG block and a ˜20 kDa HQ-25 block (FIGS. 17 and 19).

The sugar/HQ-25 diblock polymers (GlcHQ and GalHQ) were synthesized via sequential RAFT. The sugar monomers were both synthesized via an amidation reaction involving acryloyl chloride and D-glucosamine hydrochloride or D-galactosamine hydrochloride (FIG. 17). The resulting monomers, 2-deoxy-2-acrylamido glucopyranose (AAGlc) and 2-deoxy-2-acrylamido galactopyranose (AAGal) bore acrylamide functional groups as the active radical site in polymerization as opposed to the acrylate functional groups of HEA and HQ. Given that the reactivities of the propagating species were slightly different, the best results where when polymerizing the acrylate block first followed by the acrylamide block when using DDMAT as the CTA. Hence, HQ and HEA were copolymerized to generate ˜20 kDa HQ-25. This polymer was then chain extended with AAGlc or AAGal to form the diblock polymers GlcHQ and GalHQ with molar masses of ˜40 kDa (FIG. 17 and FIG. 19).

Polymers were characterized by using ¹H NMR spectroscopy to determine the molar incorporations of the various monomers. Most of the molar masses for the polymers discussed in this work were determined using size exclusion chromatography (SEC), except for GlcHQ and GalHQ. These polymers were only compatible with columns on an SEC system with an aqueous mobile phase (pH˜5). In this mobile phase there were issues with micellization/aggregation of these diblock polymers leading to overestimated molar masses and artificially increased dispersities. Hence, the molar mass calculated using ¹H NMR spectroscopy was used for calculations and is reported in FIG. 19. The pKₐ values for the diblock polymers synthesized in this work were measured via potentiometric titration and were very close to the previously measured pKₐ values of the HQ-X polymers (6.2-6.6) (FIG. 19). The fact that these polymers possess pKₐ values below physiological pH (7.4) indicates that there is a fraction of unprotonated amine groups that can buffer the endolysosomal environment and aid in transfection.

Polyplex formation and characterization. With all the components for the binary blends synthesized, colloidally stable polyplexes were formulated and screened for mRNA delivery efficiency. Previous results indicated that the order of mixing the components (HQ-X, diblock stabilizer, and nucleic acid payload) did not matter for particle size or transfection efficiency. Thus, one mixing procedure was used to form all the polyplexes in this work. First, the polymer components (HQ-X and diblock stabilizer) were mixed at various stabilizer percentages in 3% acetic acid to ensure they were fully dissolved. All the stabilizer percentages presented in this work represent the contribution of the diblock stabilizer (GlcHQ, GalHQ, or PHQ) to the overall N/P ratio, or the molar ratio of quinuclidine nitrogen atoms to phosphate groups in mRNA. For instance, at N/P=16, a 10% stabilizer incorporation would indicate that HQ-X contributes to 14.4 units of that ratio and the HQ in the stabilizer accounts for the remaining 1.6. After the polymers were mixed, they were diluted to the working concentration for N/P=16, combined with the mRNA payload in a 1:1 ratio, and allowed to anneal (final acetic acid concentration ˜0.15%, pH˜2.6). After this annealing time, the pH was brought closer to physiological pH by the addition of buffer. Two buffers were selected for this: Opti-MEM and PBS+50 mM NaHCO₃. The former is commonly used for in vitro transfections and is most relevant for the transfection results presented in this report. The latter was selected because PBS is a more universal buffer, and with the inclusion of 50 mM sodium bicarbonate the pH of the final polyplex solution rose to 7.4 (in contrast to the final pH of 6.7 for polyplexes diluted with Opti-MEM). Hence, this buffer system is more relevant for future in vivo experimentation. Interestingly, we found that particle formation for these binary mixtures of polymers could be accomplished by relying only on the electrostatic complexation with mRNA, as we observed particle formation in acidic water in the presence of mRNA but not in its absence. Although this electrostatic complexation certainly plays a role, the hydrophobic collapse of the HQ-containing portions of these formulations may be a driver of particle formation. This is because particles of similar size could be generated both with and without mRNA payload when buffer was introduced and the pH was increased above the pKₐ of the polymers.

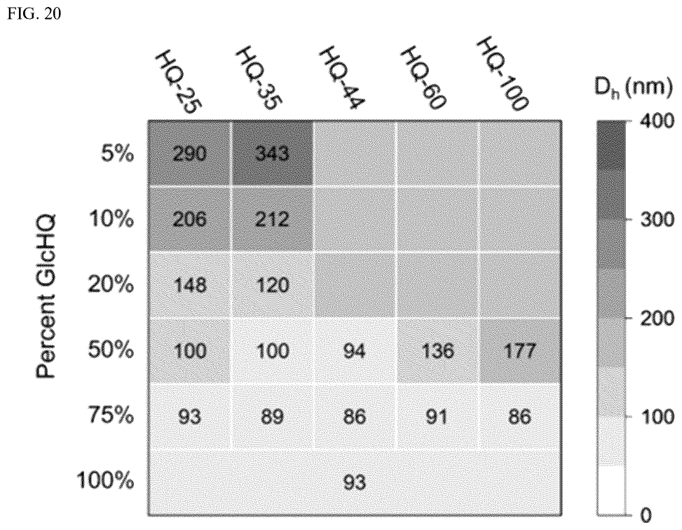

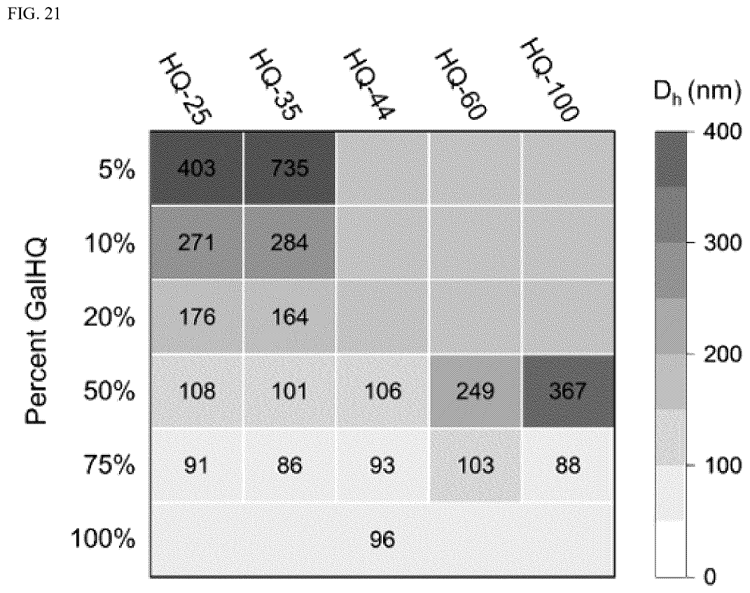

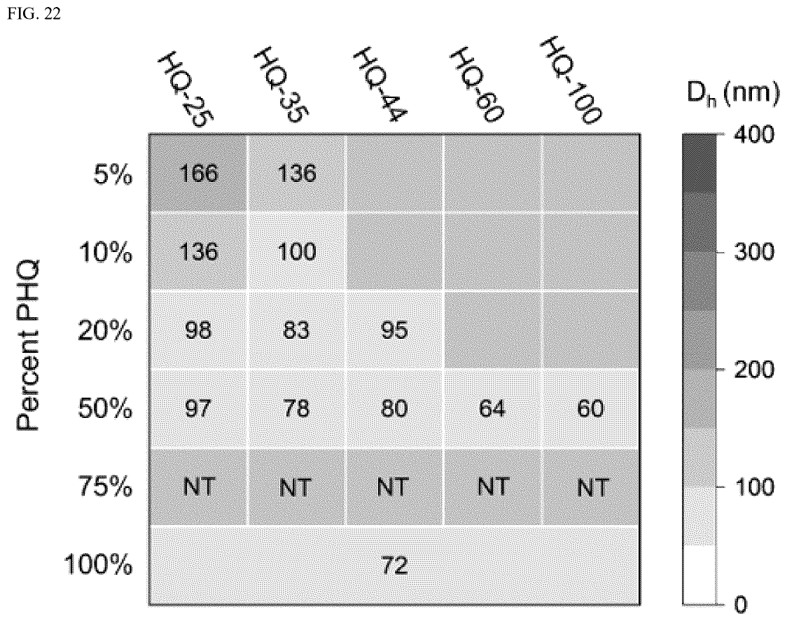

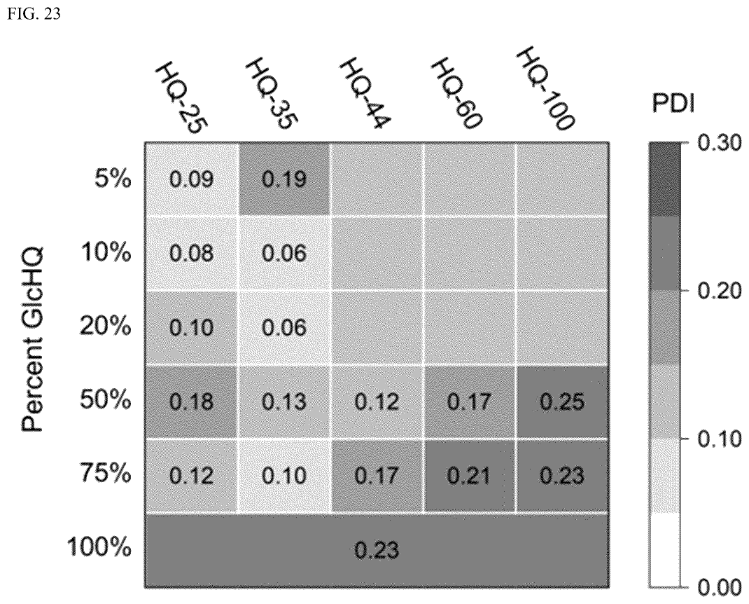

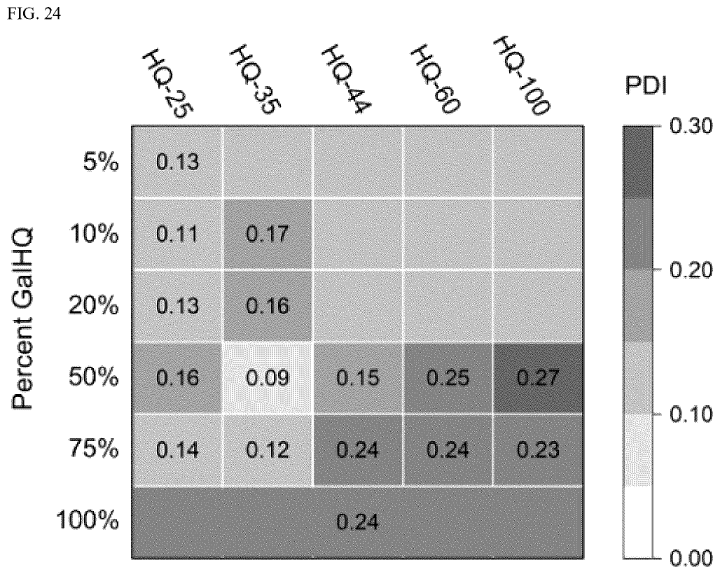

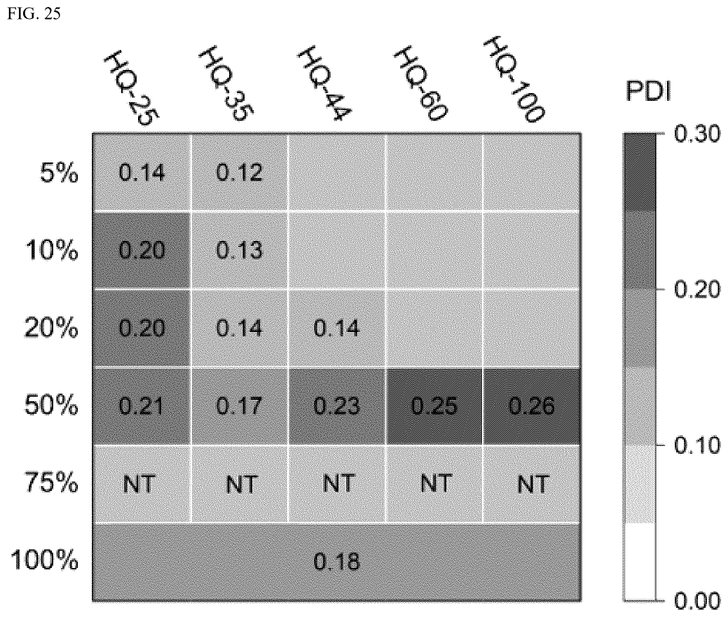

To understand the effects of the diblock stabilizers on colloidal stability, a large library of formulations consisting of five HQ-X ratios (HQ-25, 35, 44, 60, and 100) and five stabilizer percentages (5%, 10%, 20%, 50%, 75%, and 100%) was scanned using dynamic light scattering (DLS) to measure the size and distribution of the particles (FIGS. 20, 21, 22, 23, 24, and 25) after dilution in Opti-MEM. The 75% formulations were not tested for PHQ because previous results pointed to the fact that PHQ incorporations above 50% would may hamper internalization and leave these formulations inactive for nucleic acid delivery. Not every combination of HQ-X and diblock stabilizer resulted in acceptable particles. Generally, the more HQ present in the HQ-X component of the formulation, the more stabilizer was required to generate submicron particles that did not aggregate. For instance, HQ-60 and 100 could not form small, stable particles at stabilizer percentages less than 50%. Nearly all the formulations that did not aggregate resulted in particles that were <300 nm in diameter (FIGS. 20, 21, and 22). As the stabilizer percentage increased, the size of the particles also steadily decreased, giving tunable particles sizes between 70 and 300 nm. The particle size distribution of the stabilized polyplexes was generally monomodal and had polydispersity index (PDI) values that were all <0.3, with many formulations resulting in very narrowly dispersed populations (PDI≤0.2) (FIGS. 23, 24, and 25). When comparing the physical properties of the different stabilizers, the PEG-based diblock (PHQ) was able to generate smaller particles than the sugar diblock polymers. Additionally, GalHQ seemed to be slightly worse at creating small, uniform particles in comparison to GlcHQ. The total weight fraction of HQ in each formulation was examined to determine a stability threshold for these mixtures. For PHQ, the stability threshold was around 71% HQ; 5% higher than the threshold of 66% for the sugar diblocks. It is reasonable to assume that any formulations made below these thresholds would yield small, stable particles in Opti-MEM. Overall, 47 of the 73 tested formulations resulted in small, uniform particles that did not aggregate after incubation in Opti-MEM and could be brought forward.

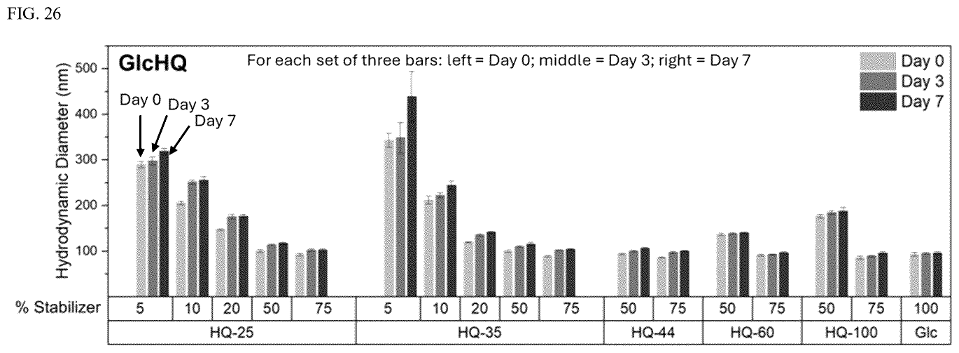

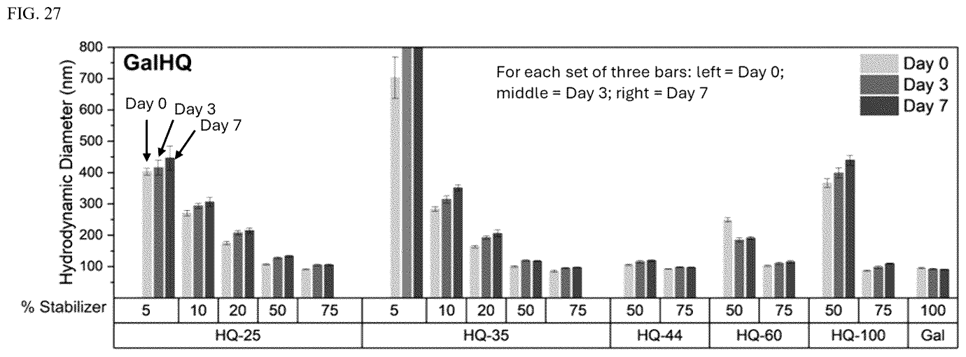

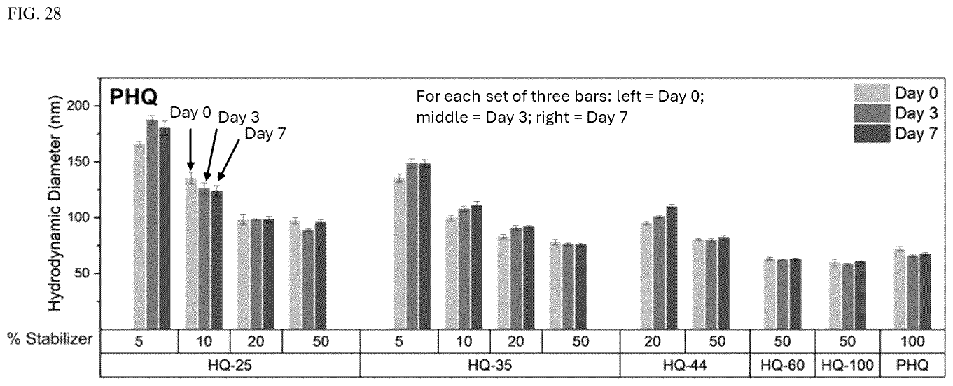

Colloidal stability over time was explored. The polyplex solutions were stored in Opti-MEM at 4° C. for one week, measuring their initial size at formulation, then three and seven days later (FIGS. 26, 27, and 28). Across the board, there were little differences in size right after formulation and after seven days (average difference=+22 nm for GlcHQ and GalHQ, +3 nm for PHQ), which demonstrates the colloidal stability of these formulations. The smaller particles (more stabilizer) maintained their size extremely well over the duration of the experiment, while the formulations that formed larger particles (less stabilizer) had larger increases in size. Only one formulation (HQ-35/5% GlcHQ) did not retain its colloidal stability over this seven-day window. Despite this, this formulation was still carried forward as it was able to create polyplexes that did not immediately aggregate into particles that were larger than 1 μm. PDI values were maintained within a window of approximately +0.05 from the original measurement. There was no upward trend in these values over time, which was an indication that the particles were not becoming more dispersed over time and that the particles were colloidally stable.

All these particle size and stability measurements were repeated in the more universal buffer of PBS+50 mM NaHCO₃ to show the behavior of this system at physiological pH. The same subset of formulations formed stable polyplexes as in Opti-MEM. Generally, in PBS, the extremes were amplified, with the lower stabilizer formulations giving larger particles and the higher stabilizer formulations giving smaller particles than in Opti-MEM. Additionally, the measurements over time showed that all the formulations except for HQ-35/5% GalHQ and GlcHQ were colloidally stable over one week, with little change to PDI. Taken as a whole, the DLS results demonstrate the ability to form colloidally stable polyplexes using binary mixtures of HQ-X and diblock stabilizers (PHQ, GlcHQ, and GalHQ) at different pH's and buffer compositions. Five HQ-X compositions (HQ-25, 35, 44, 60, and 100) and six stabilizer percentages (5%, 10%, 20%, 50%, 75%, and 100%) were mixed and of these 73 formulations, 47 remained as submicron particles with all but one of these retaining their size over a week. The majority of these formulations were <200 nm in diameter with uniform DPI's<0.2 in both Opti-MEM and PBS buffer.

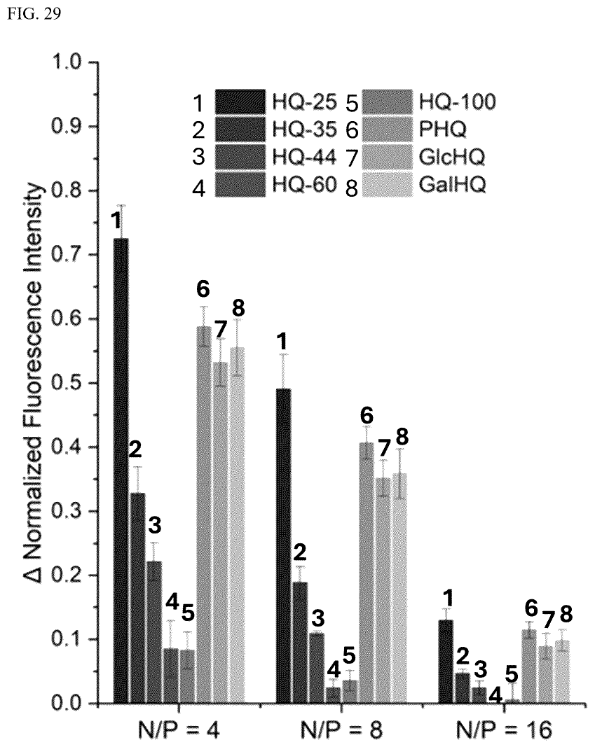

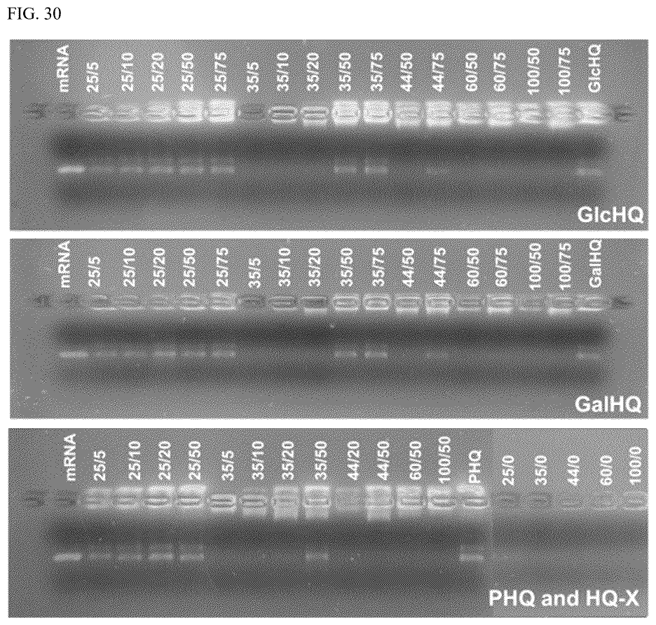

The 47 formulations that had been investigated for colloidal stability were then moved to the next stage of experimentation: understanding their mRNA binding capabilities. A RiboGreen dye exclusion assay with these formulations at N/P=16 revealed that all the formulations bound to the payload very effectively. Nearly all the RiboGreen dye was displaced by polymer in the assay, giving normalized fluorescence intensities less than 10% that of the naked mRNA control both acidic water and Opti-MEM. While these results were encouraging to show the polymers could effectively bind to and compact the mRNA payload, no trends in the binding were observed amongst the formulations. To elucidate trends, the polymer components of the binary mixtures were analyzed separately at three N/P ratios (4, 8, and 16). The lower N/P ratios were included to better illustrate any trends since the N/P=16 samples bound tightly. The normalized fluorescence intensity was measured in the acidic water prior to buffer addition, as well as after the addition of Opti-MEM. The difference in these normalized intensity values (FIGS. 29 and 30) can be used as a proxy for how tightly the polymers hold on to the payload after neutralizing the pH and adding in various salts. The results confirmed previous findings that increasing the amount of HQ in the HQ-X polymers leads to tighter mRNA binding. Also, the results indicated that HQ-25 binds similarly to the payload as the diblock stabilizers. Increasing the amount of stabilizer in the formulation seems to decrease the binding strength of the formulation, as HQ-25 bound the weakest to the mRNA payload.

To complement the dye exclusion results, a gel electrophoresis assay was used. As part of this assay, the mRNA in the polyplexes needed to be at a higher concentration to be visible in the gel. Increasing the amount of polymer and mRNA added to the solution was not done because it would necessitate a large amount of acetic acid be added to the solution and risk potential degradation of the mRNA. Thus, a procedure for concentrating the polyplexes after they had already been formed using Amicon centrifugal filtration units was used. The polyplexes were concentrated to ˜10× their typical concentration and a fraction measured on DLS to ensure that the particles were still giving the same size as unconcentrated particles. The rest of the polyplexes were run on an agarose gel for the electrophoresis assay. Unexpectedly, multiple formulations were found that have a free mRNA band, which is typically indicative of incomplete binding (FIG. 30). This contrasts with the initial dye exclusion screening in which all the formulations bound well to the mRNA. However, inspecting which formulations had bands of free mRNA confirmed the results of the second round of RiboGreen dye exclusion. Increasing the HQ content in the HQ-X portion of the formulation led to tighter binding, while increasing the stabilizer percentage caused a decrease in the binding strength. Formulations with either high stabilizer percentages of stabilizer, low X in HQ-X, or both showed free mRNA bands. The images of the gels are presented in color to also show the fluorescence of quinine. When bound to genetic material, the fluorescence of quinine is quenched, thus the wells with little quinine fluorescence (blue glow) and no free band of mRNA indicated exceptionally strong binding. All these ideas are exemplified by the HQ-35 series of formulations in FIG. 30. When compared to the lower HQ-X formulation, HQ-25, these formulations bound tighter because fewer bands of free mRNA are present. As the percentage of stabilizer increases in this series, the quinine fluorescence in the well increases and free mRNA bands begin to appear at 50%, indicating weaker binding. Collectively, the dye exclusion and the gel electrophoresis assays clearly demonstrate increased binding strength as X in HQ-X increases and at lower stabilizer incorporations. However, there was still the conflicting data point of the first round of dye exclusion showing tight binding across the board and the many instances of free mRNA bands in the gel electrophoresis assay. To understand this better, the dynamics of the system were analyzed using a Forster resonance energy transfer (FRET) assay.

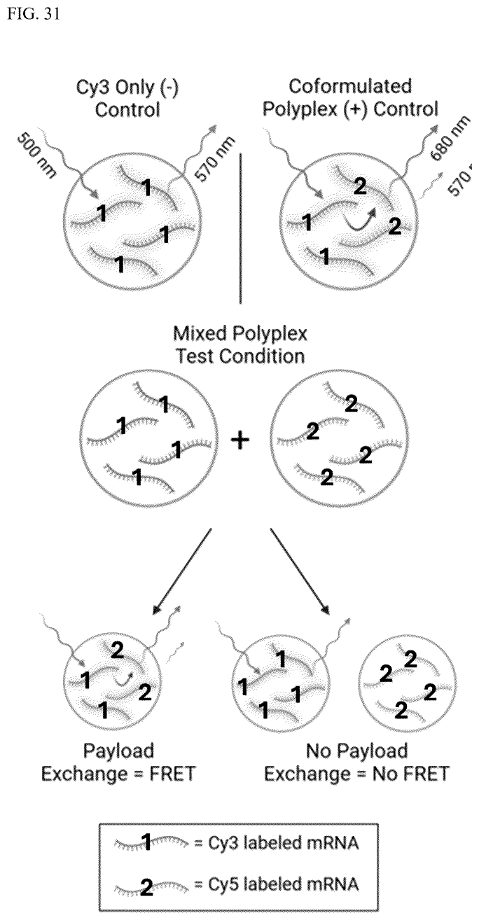

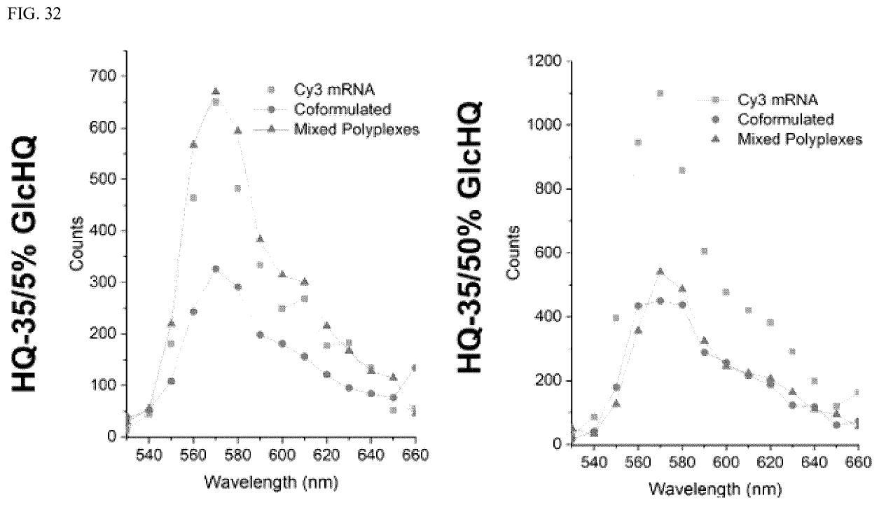

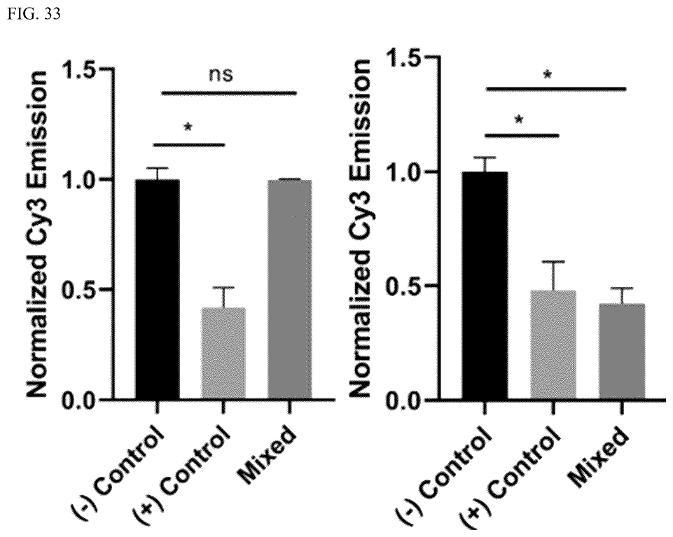

A FRET assay was employed to make better sense of the binding results discussed in the previous subsection by studying the payload dynamics in the system. Like the gel electrophoresis assay, the mRNA needed to be concentrated ˜10× to give adequate signal and was done so after polyplex formation using Amicon centrifugal filtration units. The assay was designed to investigate payload exchange within the polyplexes by utilizing a Cy3-Cy5 FRET pair. The design of the experiment is outlined in FIG. 31. A negative control with only Cy3-labeled mRNA was used to obtain the Cy3 fluorescence when no FRET is occurring. A positive control of polyplexes co-formulated with half Cy3-labeled mRNA and half Cy5-labeled mRNA was used to obtain the Cy3 fluorescence when FRET does occur and most of the Cy3 fluorescence is transferred to the Cy5 label. The test condition included polyplexes that were separately formulated with Cy3 and Cy5-labeled mRNA then mixed. If the fluorescence intensity decreased when compared to the negative control, that would demonstrate that FRET is occurring as a result of interparticle payload exchange. This is highlighted when comparing a formulation that bound strongly (HQ-35/5% GlcHQ) to a formulation that bound weakly (HQ-35/50% GlcHQ) in FIGS. 32 and 33. In the strongly binding formulation, the mixed condition gave a similar Cy3 emission spectrum as the negative control, indicating no FRET, while for the weakly binding formulation the mixed condition gave a Cy3 emission spectrum that resembles the positive control, indicating FRET by way of payload exchange.

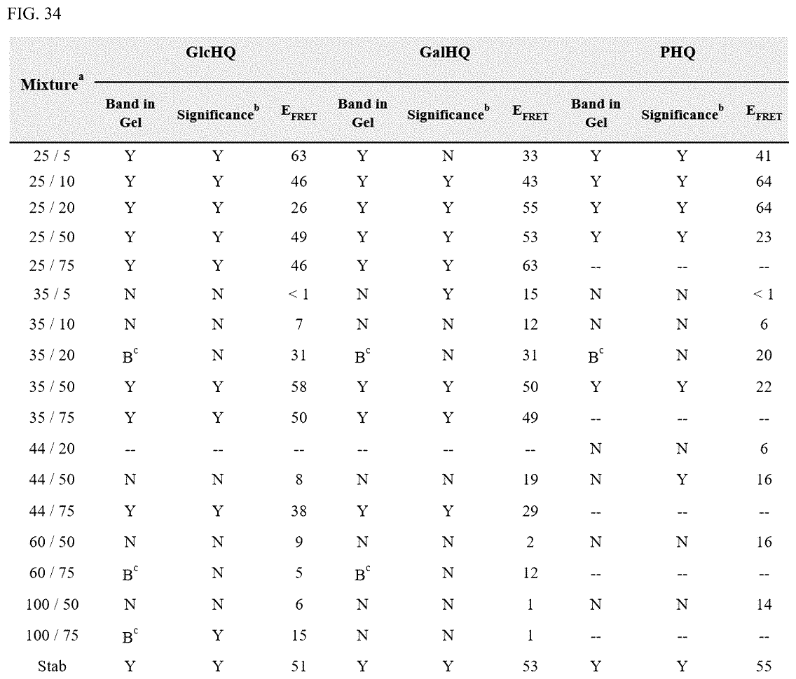

The results of the FRET assay are summarized in FIG. 34, alongside a summary of the gel electrophoresis results. For the vast majority the formulations, gel electrophoresis and FRET assays agreed. When there was a distinct band present in the gel electrophoresis assay, a significant decrease in the intensity of the Cy3 emission in the FRET assay (payload exchange occurring) was typically observed. This means that the formulations that had a free band of mRNA did not necessarily have such because the mRNA could not be bound by the polymers. Instead, those weaker binding mixtures may result in dynamic systems that could readily exchange payload and only released the mRNA because of the electrophoretic driving force in the gel mobility assay. This would resolve the discrepancy between the dye exclusion assay that showed effective compaction of the payload and the free mRNA bands seen in the gel electrophoresis assay. Lastly, the final column for each formulation in FIG. 34 quantifies the relative amounts of payload exchange using FRET efficiency (EFRET), with higher numbers corresponding to increased exchange. Generally, the weaker the binder, the higher the EFRET. Taken as a whole, the binding results indicate three main points: (1) all the formulations can effectively bind mRNA as indicated by the RiboGreen dye exclusion assay with all formulations at N/P=16; (2) lower X in HQ-X and increased stabilizer percentages lead to weaker binding, and; (3) formulations that demonstrate free mRNA in the gel electrophoresis assay do so because they are dynamic systems that can readily exchange payload, not because of incomplete binding to mRNA.

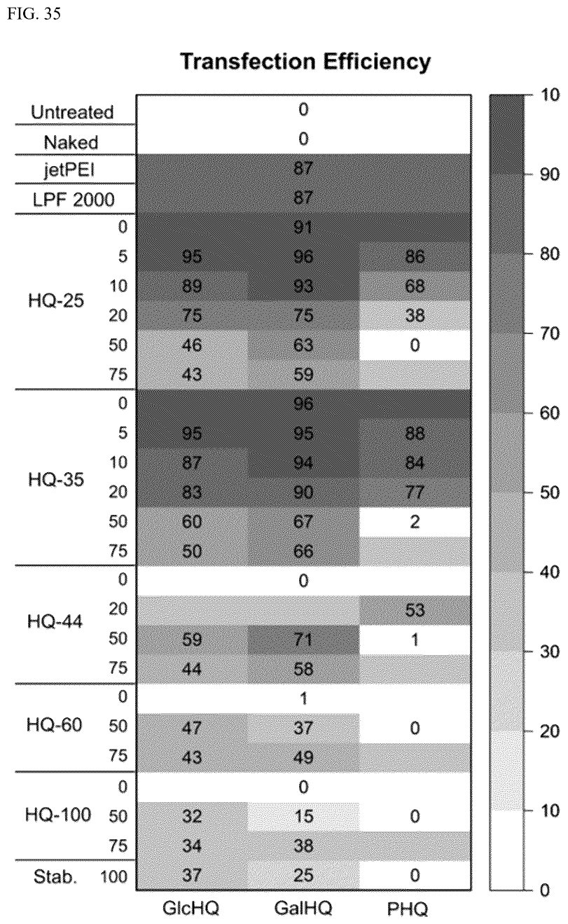

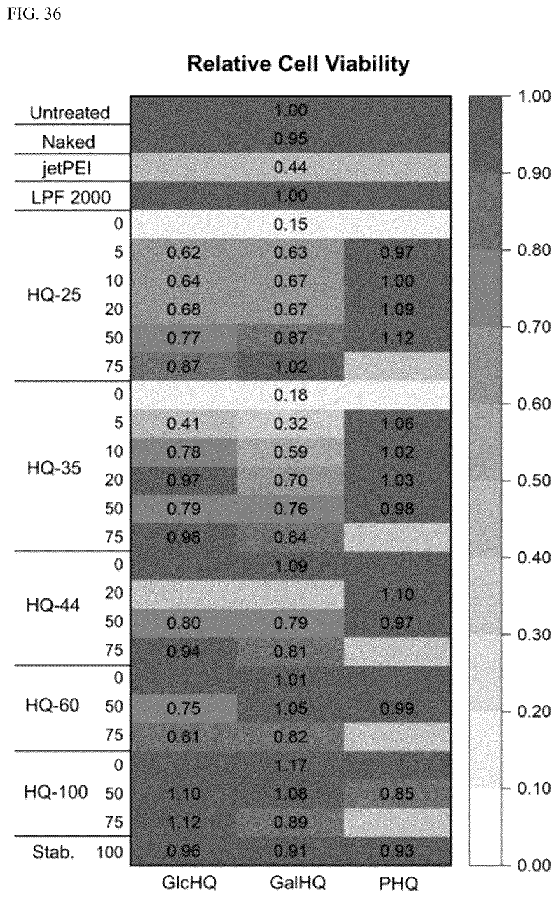

The binary mixtures were evaluated for their mRNA delivery performance using a transfection reporter assay in which mRNA encoding for enhanced green fluorescent protein (EGFP) was the payload. The 47 formulations from previous studies were used at N/P=16 to transfect human embryonic kidney cells (HEK-293T), a common benchmark cell line for discerning transfection efficiency. The results for this transfection can be seen in FIGS. 35 and 36 with the transfection efficiency (% live cells EGFP+) as determined by flow cytometry and the relative cell viability determined with a cell counting kit 8 (CCK-8) assay. The transfection efficiencies and cell viabilities of the HQ-X polymers alone (0% stabilizer) match previously reported values. HQ-25 and 35 alone can transfect cells extremely well (>90% EGFP+); however, they came with high toxicity (<20% cell viability). This has been attributed to the aggregation of the polyplexes. On the other hand, HQ-44, 60, and 100 by themselves cannot transfect cells at all, which has been attributed to binding too tightly to the mRNA. The final row of each heat map in FIGS. 35 and 36 shows the performance of the 100% stabilizer formulations without any HQ-X. These single polymer conditions serve as benchmarks for the binary mixtures of stabilizer and HQ-X. The binary mixtures gave much better combinations of transfection efficiency and cell viability than the HQ-X polymers or the diblock polymers by themselves. For instance, mixing HQ-25 with 5% GlcHQ or GalHQ gave even higher transfection efficiency (91% vs 95-96%) while also increasing the viability over four-fold (15% vs 62-63%). Additionally, these results highlight that two non-functional polymers (0% EGFP+), such as HQ-44 and PHQ, can be mixed together to give a formulation with 53% transfection efficiency and cell viability near 100%.

When examining trends in the stabilized formulations, adding more stabilizer resulted in greatly improved viability (FIG. 36), but the transfection efficiency steadily declined (FIG. 35). This trend in transfection efficiency was especially obvious in the case of the PHQ stabilized formulations. At 50% PHQ content there was a steep drop in transfection efficiency down to near 0% EGFP+, similar to the results previously obtained using this system to transfect with pDNA. This problem did not afflict the sugar diblock stabilized formulations, as even the 100% diblock formulations of pure GlcHQ and GalHQ demonstrated moderate transfection efficiencies of 37% and 25%, respectively. Because this drop off in transfection efficiency was not nearly as pronounced, the sugar diblock stabilizers had many more formulations that successfully transfected cells than PHQ. For GlcHQ and GalHQ, 14/17 formulations (82%) gave transfection efficiencies with >35% transfection and >60% viability, while for PHQ only 7/13 formulations (54%) are above these thresholds. This would fall to 7/18 formulations (39%) if we make the informed assumption that the 75% PHQ formulations would also show no transfection effects. The one area that PHQ outperformed the sugar diblock polymers was in the viability gains. While GalHQ and GlcHQ did substantially increase the viability of the cells with just 5% incorporation compared to the unstabilized HQ-X polymers (4-fold increase for HQ-25 and 2-fold increase for HQ-35), it required more stabilizer in the formulations to reach the 100% viability demonstrated by every PHQ-containing formulation. Viability trends were reinforced by a hemolysis assay, which demonstrated that including more stabilizer in the formulation improved hemocompatibility.

Lastly, when comparing the formulations presented in this paper to commercial controls, many of the formulations give similar or better results. For jetPEI (87% EGFP+), a polymer-based commercial transfection agent, 16 formulations gave statistically similar or better transfection efficiencies. All these formulations also gave statistically similar or better cell viabilities. When compared to Lipofectamine (LPF) 2000 (87% EGFP+), a lipid-based commercial transfection agent, the same 16 of the formulations gave statistically similar or better transfection efficiencies than this control. However, only 6 of these 16 formulations gave statistically similar cell viability. Collectively, these transfection results demonstrate the remarkable potency of these quinine-containing binary mixtures as mRNA delivery agents, even when compared to flagship commercial controls. The sugar diblock stabilizers showed a much wider breadth of hit formulations compared to PHQ. Increasing the PHQ content above 50% completely killed performance, while no such ceiling existed for GlcHQ and GalHQ.

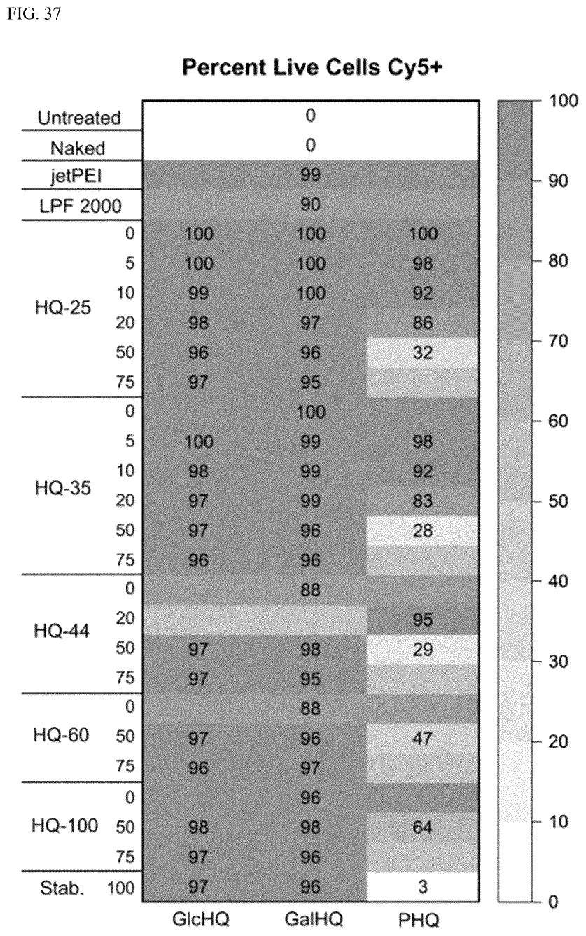

Internalization of the mRNA payload was studied in tandem with the transfection reporter assay using Cy5-labeled EGFP mRNA. The fluorescent tag on the mRNA gave insights into how effectively payload was able to make it inside of the HEK-293T cells. In terms of getting at least one copy of the mRNA payload into cells, the sugar diblock stabilized formulations all excelled with >95% of the cells positive for Cy5 fluorescence (FIG. 37). This was not the case for the PHQ mixtures. Generally, for formulations with ≥50% PHQ incorporation, less than half of the cells expressed even one copy of the mRNA payload. This explains the wall at 50% PHQ incorporation that reduced transfection efficiency to near zero, and why this wall was not present for GlcHQ and GalHQ stabilizers.

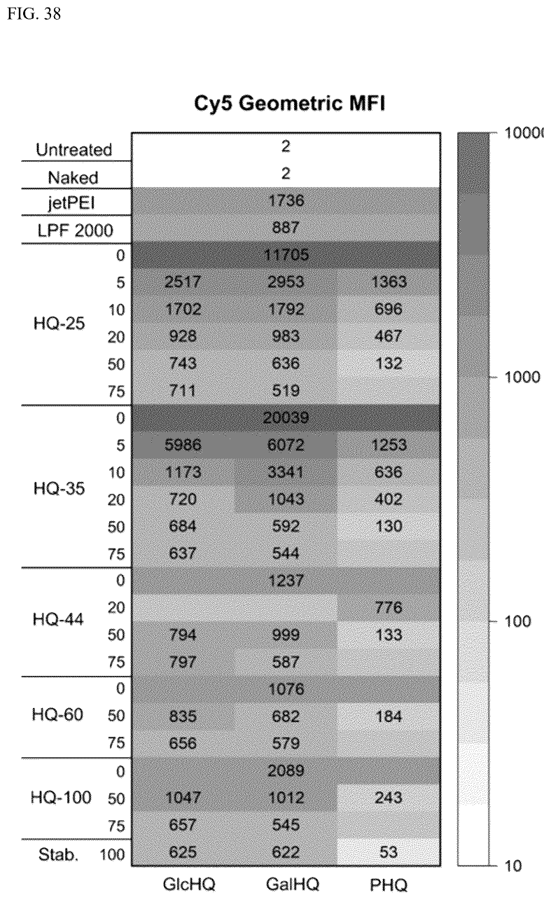

Examining only the percentage of cells with one copy of payload does not provide the complete picture, however. The amount of payload that gets into the cells at a populational level was analyzed by examining the Cy5 geometric mean fluorescence intensity (MFI) (FIG. 38). These results showed that increasing the stabilizer percentage in the binary mixtures decreased the amount of payload that was internalized. Simply adding 5% stabilizer to the HQ-25 and 35 polymers decreased the Cy5 MFI by an order of magnitude, signaling that much less payload was making it into the cells. This explains why increasing the stabilizer percentages in the formulations resulted in decreased transfection efficiencies. When looking at this metric for internalization, we can still see a distinct advantage of the sugar stabilizers compared to PHQ. For the 100% stabilizer conditions, GlcHQ and GalHQ had Cy5 MFI values that were >10× the value for PHQ. In fact, when comparing the sugar diblock polymers to PHQ for any given formulation, the Cy5 MFI was anywhere from 2-10 times higher for the sugars. It is clear that the screening effects that are consistently observed with PEGylated systems are not nearly as prevalent when utilizing glycopolymers as the hydrophilic stabilizers.

After transfecting a model cell line in HEK-293T cells, transfection trends in other cell types were investigated to demonstrate the cell type specificity imparted by the sugar diblock stabilizers. Three other cell lines were added in addition to the HEK-293T cells: A549 (human lung adenocarcinoma cells), HuH-7 (human hepatoma cells), and MDA-MB-231 (human breast adenocarcinoma cells). A549 and HuH-7 cells were chosen because of their overexpression of galectins. Both these cell lines have been used to demonstrate galactose-based targeting effects. Hence, the GalHQ-stabilized particles were predicted to perform better than the GlcHQ and PHQ-stabilized particles in these cell lines. MDA-MB-231 cells are known to overexpress mannose receptors and are often used to demonstrate mannose-based cellular targeting. Thus, in both these and HEK-293T cells, which do not have any sort of documented sugar preference, no difference between GlcHQ and GalHQ was expected in terms of transfection performance.

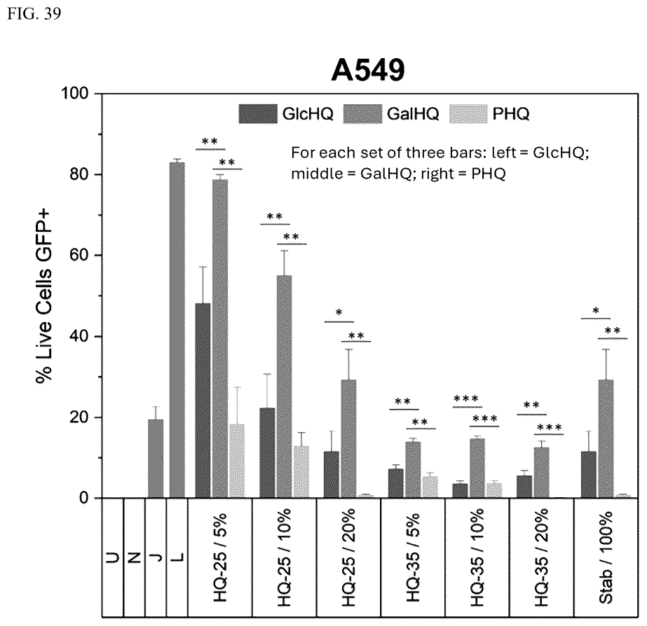

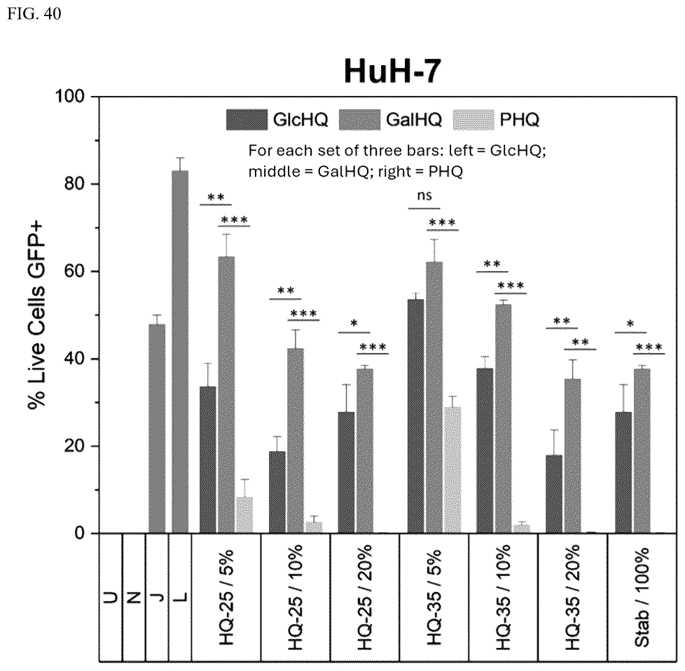

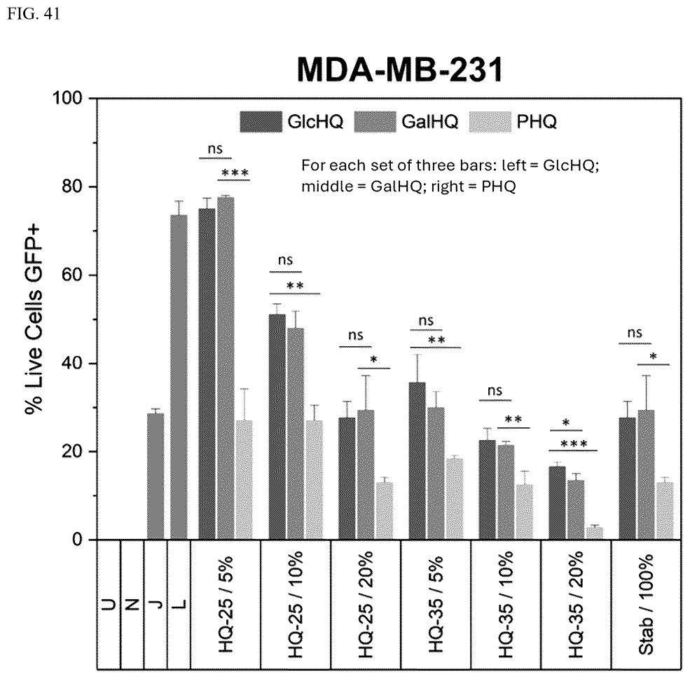

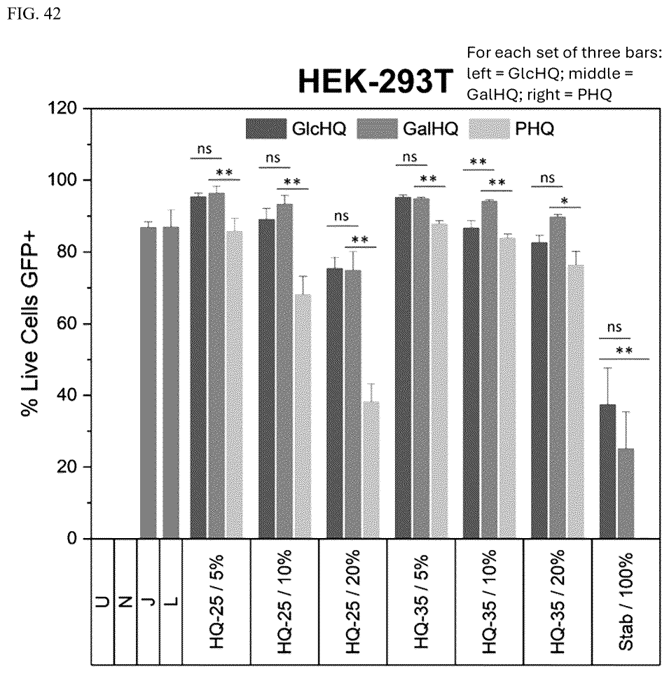

Cells were transfected with the six highest transfection efficiency formulations from HEK-293T cells: HQ-25 and HQ-35 with 5, 10, and 20% stabilizer. This was done to maximize transfection efficiencies in these cell lines that are more difficult to successfully transfect. The 100% stabilizer formulations were also included because these would have the most sugar moieties present of any of the conditions. The transfection results matched the hypotheses surrounding sugar specificity of the four cell lines. In A549 cells all seven formulation ratios tested showed significantly higher transfection for the GalHQ-stabilized mixtures compared to the GlcHQ mixtures at the same formulation ratios (FIG. 39). In this cell line, the best performing formulation (HQ-25/5% GalHQ) showed similar transfection efficiency to LPF 2000 (83% vs 79%, respectively) and a 4-fold increase over jetPEI (19%). In HuH-7 cells, there was also a strong preference for GalHQ-stabilized mixtures, as six of the seven formulations demonstrated a significant difference between the GalHQ and GlcHQ formulations at the same ratio (FIG. 40). The transfection efficiency of the best performing formulation, HQ-25/5% GalHQ, was lower than LPF 2000 in these cells (83% vs 63%, respectively), but still gave better results than jetPEI (48%). In contrast to the previously mentioned cell lines, the mannose-specific MDA-MB-231 cells did not show a preference for GlcHQ or GalHQ with six of the seven formulations showing no statistical difference between the two (FIG. 41). In this cell line, the best performing formulation, HQ-25/5% GlcHQ, was slightly better than LPF 2000 (73% vs 78%) and showed a 3-fold improvement over jetPEI (29%). Lastly, statistical analysis of the HEK-293T results from FIG. 35 provides the same results as the MDA-MD-231 cells with six of the seven formulations having no statistical difference between GlHQ and GalHQ-stabilized particles (FIG. 42). When comparing the best performing sugar-based formulation to the PHQ formulation of the same ratio, the sugars gave statistically higher transfection efficiency in every cell type tested.

In terms of viability, there was no clear difference between GlcHQ and GalHQ in all four cell lines. Also, PHQ only showed large viability gains over the sugar-based formulations in HEK-293T cells, with the other three cell lines having similar viabilities when treated with GlcHQ, GalHQ, or PHQ-stabilized particles. The internalization of payload into these four cell types using Cy5-labeled mRNA was examined. In A549 and HuH-7 cells there was a clear preference for the GalHQ formulations, as the Cy5 MFI was statistically higher in all seven of the formulation ratios for A549 cells and 5/7 for HuH-7 cells. In HEK-293T cells the trend is flipped with 5/7 formulation ratios showing no statistical difference between GalHQ and GlcHQ stabilizers. Strangely for MDA-MB-231 cells, the GalHQ mixtures were internalized better in 5/7 formulations, just like the galactose-specific cell line, HuH-7. This is despite MDA-MB-231 lacking an increased amount of galactose-binding lectins. The increase in internalization may be driven by larger particle sizes of the GalHQ-stabilized formulations compared to the GlcHQ analogs, as it has been documented that larger particle sizes give a boost to in vitro internalization. Interestingly, these results point to the fact that the performance gains owed to galactose in A549 and HuH-7 may go beyond just internalization as the internalization advantage does not always result in boosted transfection efficiency. The intracellular machinery related to galactose metabolism may aid in unpackaging galactose-based polyplexes, as it has been shown that quinine-based polyplexes unpackage in a protein-dependent manner. Hepatoma cells have been shown to modulate their galactose metabolic pathway to overexpress several proteins in it. Lastly, the transfection efficiency boost of all the sugar diblock polymers over PHQ can be attributed to much higher internalization, as we observed statistically higher Cy5 MFI values in all formulations across all cell lines.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material. In particular, the entire contents of International Publication No. WO 2023/239900 A1 are incorporated by reference. Where a conflict exists between the instant application and a reference provided herein, the instant application shall dominate.

What is claimed is:

1. A hybrid polymer comprising:
a hydrophilic block polymerized from a hydrophilic monomer; and
a hydrophobic block polymerized from at least a quinoline-containing monomer of the structure 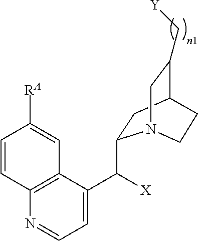
wherein:
R^{A} is H or OCH₃;
n1 is an integer from 1 to 5;
Y is H or CH₃; and
X is 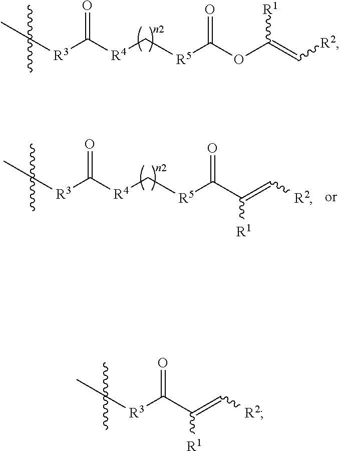
wherein:
R³, R⁴, and R⁵, are each independently O, NH, NR²⁰, or S;
wherein R²⁰ is methyl, ethyl, or propyl;
R¹ and R² are each independently H or alkyl; and
n2 is an integer from 1 to 10.

2. The hybrid polymer of claim 1, wherein X is 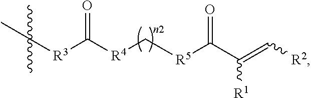
R¹ is H, R² is H, R³ is O, R⁴ is NH, R⁵ is O, and n2 is 2.

3. The hybrid polymer of claim 1, wherein R^{A} is OCH₃.

4. The hybrid polymer of claim 1, wherein n1 is 1.

5. The hybrid polymer of claim 1, wherein the quinoline-containing monomer is 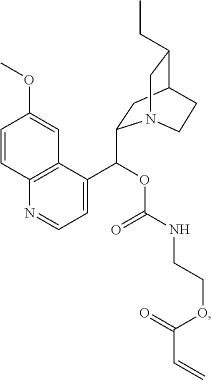 [SMILES: C=CC(=O)OCCNC(=O)OC(c1ccnc2ccc(OC)cc12)C1CC2CCN1CC2CC]
or an ionized form thereof.

6. The hybrid polymer of claim 1, wherein the hydrophobic block is a copolymer polymerized from the quinoline-containing monomer and a second monomer, wherein the second monomer is an acrylate, methacrylate, acrylamide, methacrylamide, or a vinyl monomer.

7. The copolymer of claim 6, wherein the second monomer is 2-hydroxyethyl acrylate.

8. The hybrid polymer of claim 1, wherein the hydrophilic block comprises polyethylene glycol (PEG).

9. The hybrid polymer of claim 8, wherein the hybrid polymer comprises: 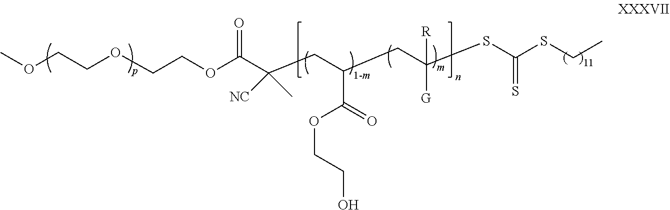
wherein:
p is 10 to 2500;
n is 10 to 1000;
m is the mol-% of the repeat group G wherein G is formed from the quinoline-containing monomer; and
R is independently H or CH₃.

10. The hybrid polymer of claim 1, wherein the hydrophilic block is polymerized from at least a carbohydrate-containing monomer.

11. The hybrid polymer of claim 10, wherein the carbohydrate-containing monomer is 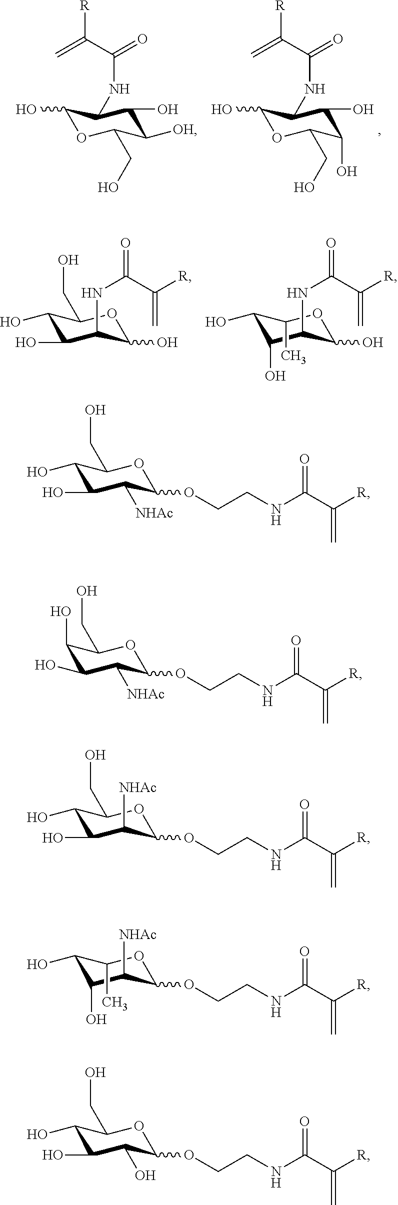 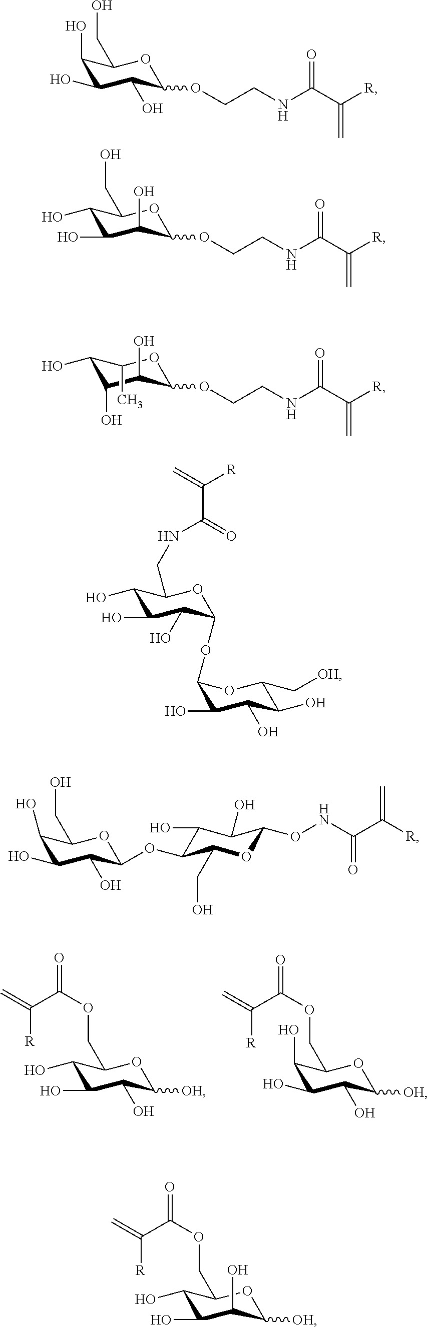
or an ionized version thereof, wherein R is H or methyl.

12. The hybrid polymer of claim 10, wherein the hybrid polymer comprises: 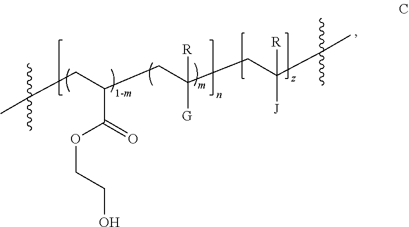
wherein:
z is 10 to 2500;
n is 10 to 1000;
m is the mol-% of the repeat group G wherein G is formed from the quinoline-containing monomer;
J is formed from the carbohydrate-containing monomer; and
each R is independently H or CH3.

13. The hybrid polymer of claim 12,
wherein G is 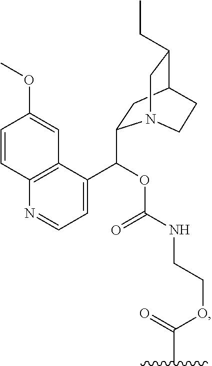 [SMILES: *C(=O)OCCNC(=O)OC(c1ccnc2ccc(OC)cc12)C1CC2CCN1CC2CC]
or an ionized form thereof.

14. The hybrid polymer of claim 11, wherein J is 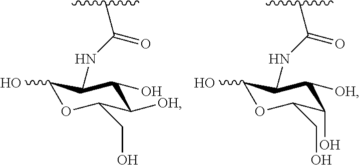
or an ionized form thereof.

15. The hybrid polymer of claim 11, wherein the hybrid polymer comprises 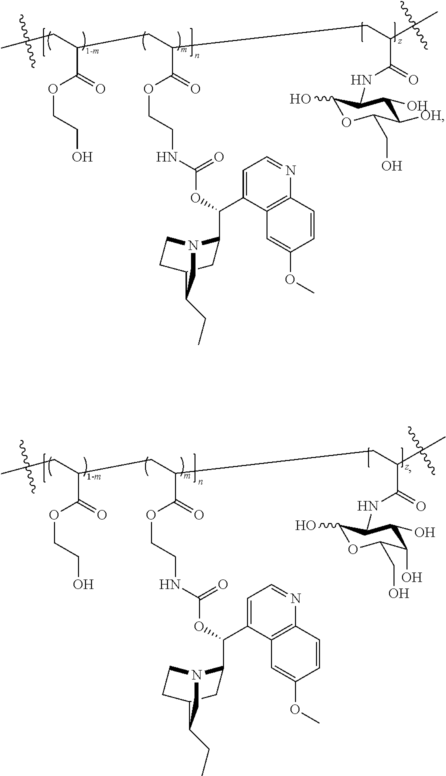
or an ionized form thereof.

16. A composition comprising:
a polyplex transfection complex comprising:
the hybrid polymer of claim 1; and
an oligonucleotide; and
a transfection medium.

17. The composition of claim 16, wherein the polyplex transfection complex further comprising a hydrophobic polymer polymerized from the same quinoline-containing monomer of the hybrid polymer and a second monomer.

18. The composition of claim 16, wherein the second monomer of the hydrophobic polymer is 2-hydroxyethyl acrylate.

19. The composition of claim 16, wherein the oligonucleotide comprises plasmid DNA or mRNA.

20. A method of transfecting a cell comprising contacting a cell with the composition of claim 16.
